(19) United States
(12) Patent Application Publication
(10) Pub. No.: US 2026/0297447 A1
(43) Pub. Date: Oct. 1, 2026

(54) FLUORINE-CONTAINING ETHER COMPOUND, LUBRICANT FOR MAGNETIC RECORDING MEDIUM, AND MAGNETIC RECORDING MEDIUM

(71) Applicant: Resonac Corporation, Tokyo (JP)

(72) Inventors: Takuya UNO, Tokyo (JP); Yutaka TANJI, Tokyo (JP)

(73) Assignee: Resonac Corporation, Tokyo (JP)

(21) Appl. No.: 19/479,835

(22) PCT Filed: Dec. 11, 2024
(86) PCT No.: PCT/JP2024/043838

§ 371 Date: Oct. 29, 2025

(30) Foreign Application Priority Data
Dec. 25, 2023 (JP) 2023-218399

(51) Int. Cl. C10M 107/44; C08G 65/26; C10N 40/18; G11B 5/725

(52) U.S. Cl. CPC C10M 107/44; C08G 65/2633; G11B 5/7253; C10M 2217/065; C10N 2040/18

(57) ABSTRACT

A fluorine-containing ether compound represented by the following formula. R¹—O—CH₂—R²—(CH₂—R³—CH₂—R²)ₙ—CH₂—O—R⁴ (R¹ is a formula (2-1) or a formula (2-2). R² is a perfluoropolyether chain. R⁴ is an organic group having at least one polar group and having 1 to 50 carbon atoms. R³ is a divalent linking group having at least one hydroxyl group. n is an integer of 0 to 2.)

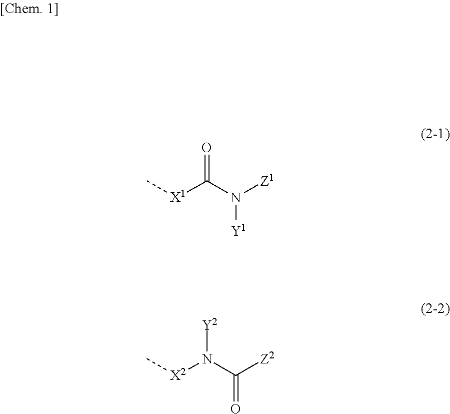

### TECHNICAL FIELD

The present invention relates to a fluorine-containing ether compound, a lubricant for a magnetic recording medium, and a magnetic recording medium.

Priority is claimed on Japanese Patent Application No. 2023-218399, filed Dec. 25, 2023, the content of which is incorporated herein by reference.

### BACKGROUND ART

In order to improve the recording density of a magnetic recording and reproducing device, development of a magnetic recording medium suitable for a high recording density is underway.

Conventionally, there have been magnetic recording media having a recording layer formed on a substrate and a protective layer of carbon or the like formed on the recording layer. The protective layer protects information recorded in the recording layer and enhances the sliding properties on magnetic heads. In addition, the protective layer coats the recording layer to prevent metal that is contained in the recording layer from being corroded by an environmental substance.

However, the durability of the magnetic recording media cannot be sufficiently obtained only by providing the protective layer on the recording layer. Therefore, a lubricating layer having a thickness of approximately 0.5 to 3 nm is formed by applying a lubricant on the surface of the protective layer. The lubricating layer improves the durability and protective force of the protective layer to prevent the intrusion of a contamination substance into the magnetic recording medium.

As the lubricant that is used at the time of forming the lubricating layer in the magnetic recording medium, for example, lubricants containing a compound having a polar group, such as a hydroxyl group or an amide group, at a terminal of a fluorine-based polymer having a repeating structure containing —CF₂— have been proposed (for example, refer to Patent Document 1 to Patent Document 5).

Patent Document 1 discloses a fluorine-containing ether compound in which divalent linking groups having a polar group are linked to both terminals of a perfluoropolyether chain, and a terminal group in which one or more hydrogen atoms of a chain-like organic group having 1 to 8 carbon atoms have been substituted with groups having an amide bond bonds to at least one of the divalent linking groups.

Patent Document 2 discloses a fluorine-containing ether compound having terminal groups including a group in which a carbonyl carbon atom or nitrogen atom of an amide bond and an aromatic hydrocarbon have directly bonded to each other at one or both of the terminals of a perfluoropolyether chain and having three or more hydroxyl groups.

Patent Document 3 discloses a method for synthesizing a fluoropolyalkylether amide that is used as a lubricant for magnetic recording media. Patent Document 3 describes a method for producing a fluoropolyalkylether amide in which amide groups directly bond to both terminals of a perfluoropolyalkylether by reacting ester groups disposed at both terminals of a fluoropolyalkylether and an amine compound having a polar group.

Patent Document 4 discloses a fluorine-containing ether compound having a skeleton in which a plurality of perfluoropolyether chains have bonded to each other through a linking group having one polar group. Patent Document 4 discloses a fluorine-containing ether compound in which a divalent linking group having at least one polar group and a terminal group have bonded in this order to each of both terminals of the skeleton through methylene. Furthermore, Patent Document 4 discloses a fluorine-containing ether compound in which at least one terminal group is a group having an amide bond.

Patent Document 5 discloses a fluoropolyether compound including three or more perfluoropolyether groups and having a perfluoroether group linked thereto by a linking group including a hydrocarbon group having at least one hydroxyl group. Patent Document 5 describes a fluoropolyether compound having a terminal group having at least one hydroxyl group at each of both terminals.

### CITATION LIST

### Patent Document

Patent Document 1: PCT International Publication No. WO 2019/039265 (A)
Patent Document 2: PCT International Publication No. WO 2023/033044 (A)
Patent Document 3: U.S. Pat. No. 6,187,954 (B)
Patent Document 4: PCT International Publication No. WO 2023/033055 (A)
Patent Document 5: PCT International Publication No. WO 2018/147017 (A)

### SUMMARY OF INVENTION

### Technical Problem

In magnetic recording and reproducing devices, there is a demand to further decrease the flying height of the magnetic head. Therefore, there is a demand to further thin the thicknesses of a protective layer and/or a lubricating layer in a magnetic recording medium.

However, generally, when the thickness of the lubricating layer is made to be thin, the chemical substance resistance and wear resistance of the magnetic recording medium tend to deteriorate. In addition, when the flying height of the magnetic head is made to be small, there is a case where pickup, the attachment of a fluorine-containing ether compound in the lubricating layer to the magnetic head, occurs.

The present invention has been made in consideration of the above-described circumstance, and an object of the present invention is to provide a fluorine-containing ether compound that enables the formation of a lubricating layer having excellent chemical substance resistance and wear resistance and being capable of curbing pickup and that can be suitably used as a material of lubricants for magnetic recording media.

In addition, another object of the present invention is to provide a magnetic recording medium having a lubricating layer containing the fluorine-containing ether compound of the present invention, in which the chemical substance resistance is favorable, the wear resistance is excellent, and pickup is curbed.

### Solution to Problem

In order to achieve the above-described objects, the present inventors repeated intensive studies.

As a result, the present inventors found that a fluorine-containing ether compound in which terminal groups have bonded through a methylene group and an ether oxygen atom to both ends of a perfluoropolyether chain or to both ends of a skeleton to which two or three perfluoropolyether chains have bonded through a divalent linking group having a hydroxyl group, in which the terminal group is a specific group including an amide bond portion (C(═O)N), in which the amide bond portion is bonded to the ether oxygen atom through a linking group including no polar groups but including one or more carbon atoms, or an organic group having at least one polar group and having 1 to 50 carbon atoms, and at least one terminal group is the above-described specific group is preferable and came to an idea of the present invention.

That is, the present invention relates to the following matters.

[1] A fluorine-containing ether compound that is represented by a following formula (1):

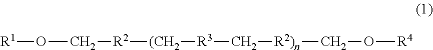

(in the formula (1), R¹ is represented by a formula (2-1) or a formula (2-2). R² is a perfluoropolyether chain. R⁴ is an organic group having at least one polar group and having 1 to 50 carbon atoms. R³ is a divalent linking group having at least one hydroxyl group. n is an integer of 0 to 2. In a case where n is 1 or 2, a part or all of a plurality of R²'s may be identical to each other or may be different from each other. In a case where n is 2, two R³'s may be identical to each other or may be different from each other.)

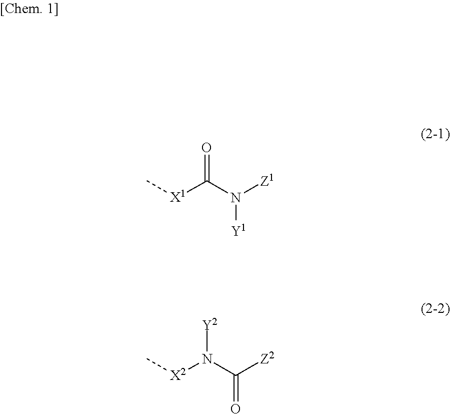

(in the formula (2-1), X¹ is a divalent linking group including no polar groups, optionally including an ether oxygen atom, and having 1 to 12 carbon atoms. Y¹ and Z¹ are each independently an organic group or a hydrogen atom, the organic group having 1 to 30 carbon atoms and optionally including at least one of a polar group and an ether oxygen atom. Y¹ and Z¹ may bond to each other to form a ring.)

(in the formula (2-2), X² is a divalent linking group including no polar groups, optionally including an ether oxygen atom, and having 1 to 12 carbon atoms. Y² is an organic group or a hydrogen atom, the organic group having 1 to 30 carbon atoms and optionally including at least one of a polar group and an ether oxygen atom. Z² is an organic group optionally including at least one of a polar group and an ether oxygen atom and having 1 to 30 carbon atoms. Y² and Z² may bond to each other to form a ring.)

[2] The fluorine-containing ether compound according to [1], in which X¹ in the formula (2-1) and X² in the formula (2-2) are each independently represented by the following formula (3).

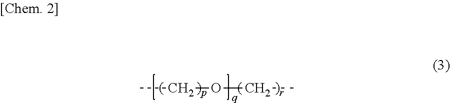

(in the formula (3), p is an integer of 2 to 4, q is an integer of 0 to 2, and r is an integer of 1 to 4. In a case where q is 2, two p's may be identical to each other or may be different from each other. A left terminal in the formula (3) is a side bonding to an oxygen atom of —R²—CH₂—O—, and a right terminal is a side bonding to a carbonyl carbon atom or nitrogen atom that configures an amide bond.)

[3] The fluorine-containing ether compound according to [1] or [2], in which at least one of Y¹ and Z¹ in the formula (2-1) is a hydrogen atom, and Y² in the formula (2-2) is a hydrogen atom.

[4] The fluorine-containing ether compound according to any of [1] to [3], in which R³ in the formula (1) is a divalent linking group represented by a following formula (4).

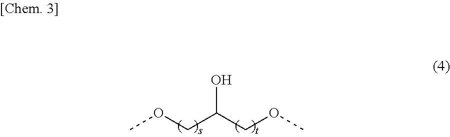

(in the formula (4), s is an integer of 1 to 3, and t is an integer of 1 to 3. An oxygen atom at a left terminal in the formula (4) bonds to —CH₂— on an R¹ side, and an oxygen atom at a right terminal bonds to —CH₂— on an R⁴ side.)

[5] The fluorine-containing ether compound according to any of [1] to [4], in which R⁴ in the formula (1) is not a group represented by the formula (2-1) or the formula (2-2), and at least one of the polar groups included in R⁴ is a hydroxyl group.

[6] The fluorine-containing ether compound according to [5], in which R⁴ in the formula (1) includes two or three polar groups, and at least one of the polar groups is a secondary hydroxyl group.

[7] The fluorine-containing ether compound according to [5] or [6], in which R⁴ in the formula (1) is any of groups represented by following formulae (5-1) to (5-3):

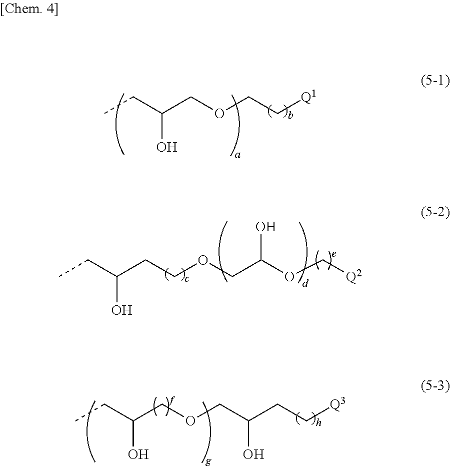

(in the formula (5-1), a is an integer of 1 or 2, and b is an integer of 0 to 3. Q¹ is an aromatic hydrocarbon group, an unsaturated heterocyclic group, an alkenyl group, an alkynyl group, or a polar group. In a case where a is 1, Q¹ is a polar group. In a case where Q¹ is an aromatic hydrocarbon group or an unsaturated heterocyclic group, an atom that configures a ring structure in Q¹ bonds to a methylene group adjacent to Q¹. In a case where Q¹ is an alkenyl group or an alkynyl group, a carbon atom that configures an unsaturated bond in Q¹ bonds to the methylene group adjacent to Q¹.)

(in the formula (5-2), c is an integer of 1 to 3, d is an integer of 0 or 1, and e is an integer of 0 to 3. Q² is an aromatic hydrocarbon group, an unsaturated heterocyclic group, an alkenyl group, an alkynyl group, or a polar group. In a case where d is 0, Q² is a polar group. In a case where Q² is an aromatic hydrocarbon group or an unsaturated heterocyclic group, an atom that configures a ring structure in Q² bonds to a methylene group adjacent to Q². In a case where Q² is an alkenyl group or an alkynyl group, a carbon atom that configures an unsaturated bond in Q² bonds to the methylene group adjacent to Q².)

(in the formula (5-3), f is an integer of 1 to 3, g is an integer of 0 or 1, and h is an integer of 0 to 3. Q³ is an aromatic hydrocarbon group, an unsaturated heterocyclic group, an alkenyl group, an alkynyl group, or a polar group. In a case where g is 0, Q³ is a polar group. In a case where Q³ is an aromatic hydrocarbon group or an unsaturated heterocyclic group, an atom that configures a ring structure in Q³ bonds to a methylene group adjacent to Q³. In a case where Q³ is an alkenyl group or an alkynyl group, a carbon atom that configures an unsaturated bond in Q³ bonds to the methylene group adjacent to Q³.)

[8] The fluorine-containing ether compound according to any one of [1] to [4], in which R⁴ in the formula (1) is represented by the formula (2-1) or (2-2).

[9] The fluorine-containing ether compound according to [8], in which R¹ and R⁴ in the formula (1) are identical to each other.

[10] The fluorine-containing ether compound according to any one of [1] to [9], in which R²'s in the formula (1) are each independently a perfluoropolyether chain represented by the following formula (6).

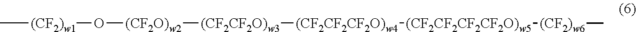

(in the formula (6), w2, w3, w4, and w5 indicate average degrees of polymerization and each independently represent 0 to 20. Here, w2, w3, w4, and w5 do not all become 0 at the same time. w1 and w6 are average values representing the numbers of CF₂ and each independently represent 1 to 3. An array order of (CF₂O), (CF₂CF₂O), (CF₂CF₂CF₂O), and (CF₂CF₂CF₂CF₂O), which are repeating units in the formula (6), is not particularly limited.)

[11] The fluorine-containing ether compound according to any one of [1] to [10], in which R² in the formula (1) is any one selected from perfluoropolyether chains represented by following formulae (6-1) to (6-4).

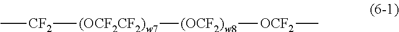

(in the formula (6-1), w7 and w8 indicate average degrees of polymerization, w7 represents 1 to 20, and w8 represents 0 to 20.)

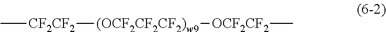

(in the formula (6-2), w9 indicates an average degree of polymerization and represents 1 to 15.)

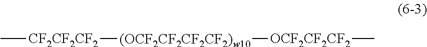

(in the formula (6-3), w10 indicates an average degree of polymerization and represents 1 to 10.)

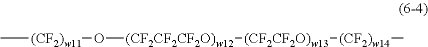

(in the formula (6-4), w12 and w13 indicate average degrees of polymerization and each independently represent 1 to 20. w11 and w14 are average values representing the numbers of CF₂ and each independently represent 1 to 2.)

[12] The fluorine-containing ether compound according to any one of [1] to [11], in which n in the formula (1) is 0.

[13] The fluorine-containing ether compound according to any one of [1] to [11], in which n in the formula (1) is 1.

[14] The fluorine-containing ether compound according to any one of [1] to [11], in which n in the formula (1) is 2.

[15] The fluorine-containing ether compound according to claim 1, in which the fluorine-containing ether compound represented by the formula (1) is any of compounds represented by following formulae (1A) to (1T) and (2A) to (2E).

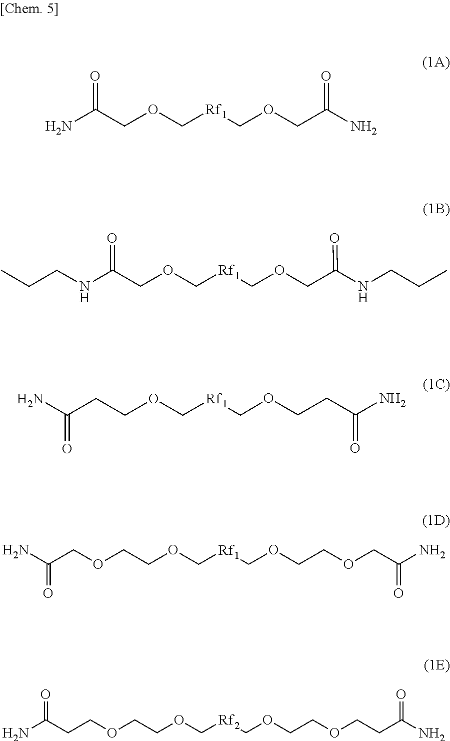

(in Rf₁ in the formula (1A), i and j indicate average degrees of polymerization, i represents 1 to 20, and j represents 0 to 20.)

(in Rf₁ in the formula (1B), i and j indicate average degrees of polymerization, i represents 1 to 20, and j represents 0 to 20.)

(in Rf₁ in the formula (1C), i and j indicate average degrees of polymerization, i represents 1 to 20, and j represents 0 to 20.)

(in Rf₁ in the formula (1D), i and j indicate average degrees of polymerization, i represents 1 to 20, and j represents 0 to 20.)

(in Rf₂ in the formula (1E), k indicates an average degree of polymerization and represents 1 to 15.)

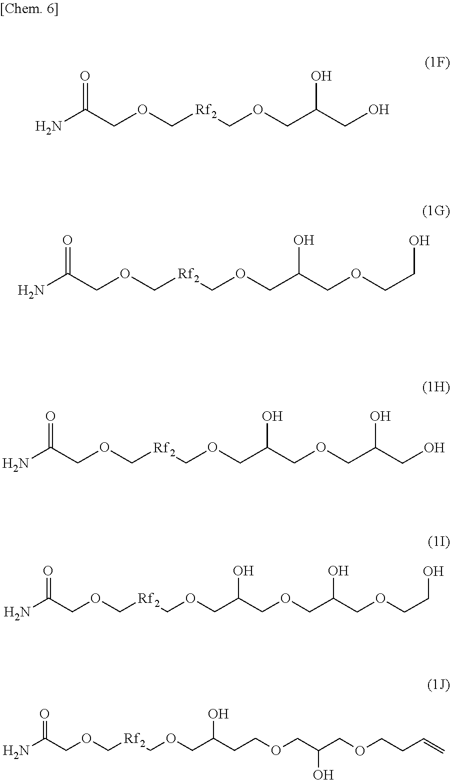

(in Rf₂ in the formula (1F), k indicates an average degree of polymerization and represents 1 to 15.)

(in Rf₂ in the formula (1G), k indicates an average degree of polymerization and represents 1 to 15.)

(in Rf₂ in the formula (1H), k indicates an average degree of polymerization and represents 1 to 15.)

(in Rf₂ in the formula (1I), k indicates an average degree of polymerization and represents 1 to 15.)

(in Rf₂ in the formula (1J), k indicates an average degree of polymerization and represents 1 to 15.)

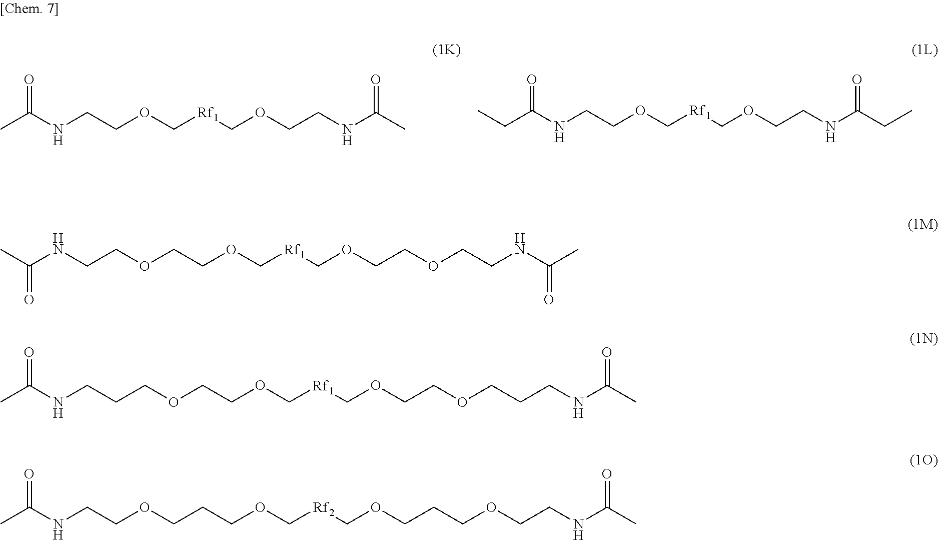

(in Rf₁ in the formula (1K), i and j indicate average degrees of polymerization, i represents 1 to 20, and j represents 0 to 20.)

(in Rf₁ in the formula (1L), i and j indicate average degrees of polymerization, i represents 1 to 20, and j represents 0 to 20.)

(in Rf₁ in the formula (1M), i and j indicate average degrees of polymerization, i represents 1 to 20, and j represents 0 to 20.)

(in Rf₁ in the formula (1N), i and j indicate average degrees of polymerization, i represents 1 to 20, and j represents 0 to 20.)

(in Rf₂ in the formula (1O), k indicates an average degree of polymerization and represents 1 to 15.)

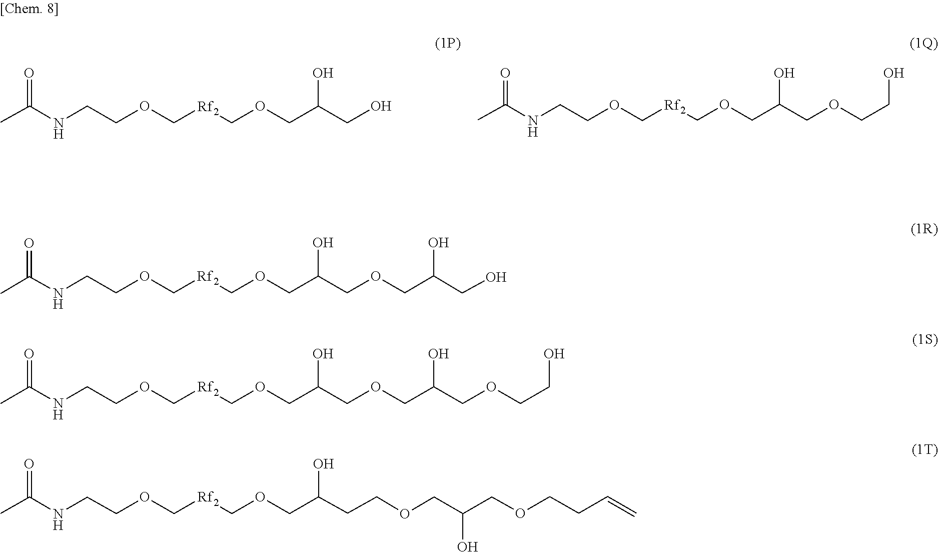

(in Rf₂ in the formula (1P), k indicates an average degree of polymerization and represents 1 to 15.)

(in Rf₂ in the formula (1Q), k indicates an average degree of polymerization and represents 1 to 15.)

(in Rf₂ in the formula (1R), k indicates an average degree of polymerization and represents 1 to 15.)

(in Rf₂ in the formula (1S), k indicates an average degree of polymerization and represents 1 to 15.)

(in Rf₂ in the formula (1T), k indicates an average degree of polymerization and represents 1 to 15.)

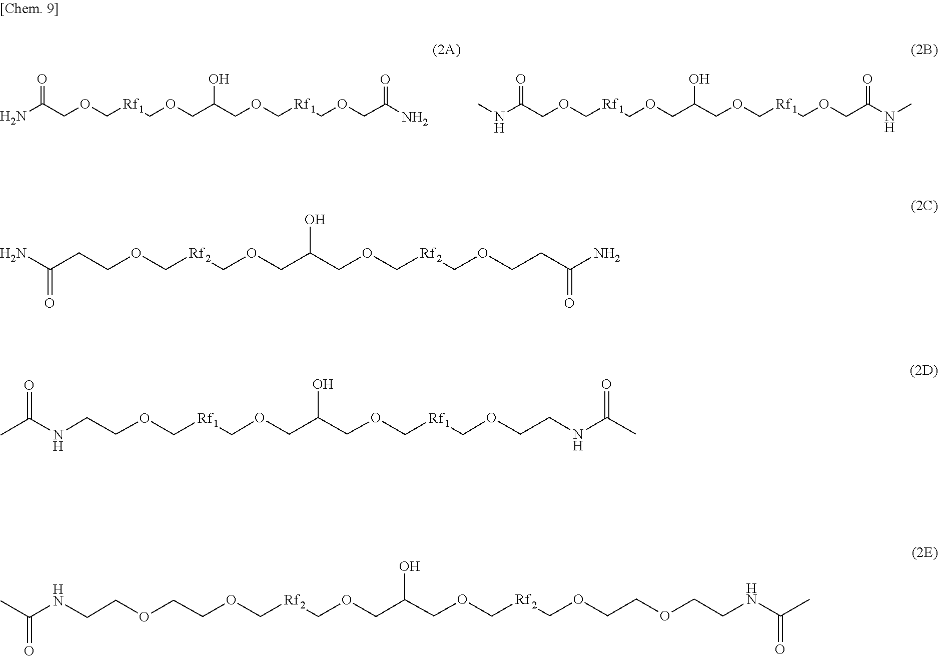

(in two Rf₁'s in the formula (2A), i and j indicate average degrees of polymerization, i represents 1 to 20, and j represents 0 to 20. In the two Rf₁'s, the average degrees of polymerization may be identical to each other or may be different from each other)

(in two Rf₁'s in the formula (2B), i and j indicate average degrees of polymerization, i represents 1 to 20, and j represents 0 to 20. In the two Rf₁'s, the average degrees of polymerization may be identical to each other or may be different from each other)

(in two Rf₂'s in the formula (2C), k indicates an average degree of polymerization and represents 1 to 15. In the two Rf₂'s, the average degrees of polymerization may be identical to each other or may be different from each other)

(in two Rf₁'s in the formula (2D), i and j indicate average degrees of polymerization, i represents 1 to 20, and j represents 0 to 20. In the two Rf₁'s, the average degrees of polymerization may be identical to each other or may be different from each other)

(in two Rf₂'s in the formula (2E), k indicates an average degree of polymerization and represents 1 to 15. In the two Rf₂'s, the average degrees of polymerization may be identical to each other or may be different from each other)

[16] The fluorine-containing ether compound according to any one of [1] to [15], in which a number-average molecular weight is within a range of 500 to 10000.

[17] A lubricant for a magnetic recording medium containing the fluorine-containing ether compound according to any one of [1] to [16].

[18] A magnetic recording medium having at least a magnetic layer, a protective layer, and a lubricating layer provided in order on a substrate,
in which the lubricating layer contains the fluorine-containing ether compound according to any one of [1] to [16].

[19] The magnetic recording medium according to [18], in which the lubricating layer has an average film thickness of 0.5 nm to 2.0 nm.

### Advantageous Effects of Invention

The fluorine-containing ether compound of the present invention is a compound represented by the formula (1) and is suitable as a material of lubricants for magnetic recording media.

The lubricant for magnetic recording medium of the present invention contains the fluorine-containing ether compound of the present invention and thus enables the formation of a lubricating layer in which the chemical substance resistance is favorable, the wear resistance is high, and pickup can be curbed even when the thickness is made to be thin.

The magnetic recording medium of the present invention has a lubricating layer containing the fluorine-containing ether compound of the present invention. Therefore, the magnetic recording medium of the present invention has favorable chemical substance resistance and excellent wear resistance, curbs pickup, and is excellent in terms of reliability and durability. In addition, the lubricating layer in the magnetic recording medium of the present invention has favorable chemical substance resistance and wear resistance and is capable of curbing pickup, and it is thus possible to thin the thickness and to decrease the flying height of a magnetic head.

### BRIEF DESCRIPTION OF DRAWINGS

FIG. 1 A schematic cross-sectional view showing one embodiment of a magnetic recording medium of the present invention.

### DESCRIPTION OF EMBODIMENTS

In order to achieve the above-described objects, the present inventors repeated intensive studies as described below.

Conventionally, as a material of a lubricant for a magnetic recording medium (hereinafter abbreviated as “lubricant” in some cases) that is applied to the surface of a protective layer, a fluorine-containing ether compound having a polar group, such as a hydroxyl group, has been preferably used. The polar group that is included in the fluorine-containing ether compound bonds to an active point on the protective layer to improve the adhesion of a lubricating layer to the protective layer. In conventional fluorine-containing ether compounds, a polar group is disposed at a terminal of the chain structure. In addition, in a case where the fluorine-containing ether compound has a plurality of perfluoropolyether chains, the polar group is also disposed between the perfluoropolyether chains adjacent to each other.

However, in a case where a lubricating layer of thin thickness is formed on a protective layer using a conventional lubricant, since the adhesion of the lubricant to the protective layer is insufficient, it is difficult to realize a lubricating layer having favorable chemical substance resistance, excellent wear resistance, and a high pickup-curbing effect.

In more detail, when the adhesion of the lubricant to the protective layer is not enough, the lubricant applied onto the lubricating layer falls into a bulky state. Therefore, the coating state of the lubricating layer on the protective layer is likely to be nonuniform. When the coating state of the lubricating layer is nonuniform, the chemical substance resistance and wear resistance of the lubricating layer become insufficient.

Therefore, in a case where the adhesion of the lubricant to the protective layer is insufficient, sufficient chemical substance resistance and wear resistance cannot be obtained unless the coating state of the lubricating layer on the protective layer is made to be uniform by thickening the film thickness of the lubricating layer. However, when the film thickness is thickened to make the coating state of the lubricating layer uniform, pickup of the lubricant by a magnetic head is likely to occur, which leads to deterioration of the reliability and the durability in some cases.

As a method for improving the adhesion of the lubricant to the protective layer, it is conceivable to use a fluorine-containing ether compound including a plurality of polar groups bonding to both terminals of a chain structure including a perfluoropolyether chain as a material for lubricants. However, in a lubricating layer formed using such a fluorine-containing ether compound, there is a case where the adhesion to the protective layer is too strong, the lubricity is thus impaired, and the wear resistance is not enough.

In addition, in a fluorine-containing ether compound having a large number of polar groups bonding thereto, since the surface free energy of the entire molecule is high, there is a case where a lubricating layer formed using this compound easily allows attachment of a chemical substance thereto. Furthermore, when the number of polar groups in the fluorine-containing ether compound is too large, some of the polar groups are likely not to bond to active points on the protective layer, and the interaction between the polar groups in the molecule and/or between molecules is less likely to occur. As a result, when the number of the polar groups that are included in the fluorine-containing ether compound is too large, it is estimated that an effect of improving the adhesion of the lubricating layer to the protective layer attributed to the polar groups included deteriorates.

Therefore, the present inventors paid attention to the kind and disposition of polar groups that are included in a fluorine-containing ether compound and the bond between the polar group and an active point on a protective layer. In addition, the present inventors estimated that when the kind and disposition of the polar groups that are included in a lubricant are appropriate, a lubricating layer that realizes a uniform coating state and excellent adhesion to the protective layer can be obtained and repeated intensive studies to realize a fluorine-containing ether compound enabling the formation of a lubricating layer having favorable chemical substance resistance and wear resistance and a high pickup-curbing effect.

As a result, the present inventors found that a fluorine-containing ether compound represented by a formula (1) is preferable.

In a lubricating layer containing the fluorine-containing ether compound represented by the formula (1), there is no case where the wear resistance is not enough due to the adhesion to a protective layer being too strong for a reason <1> to be described below, there is no case where attachment of a chemical substance is easily allowed due to the surface free energy of the entire fluorine-containing ether compound molecule being too high for a reason <2> to be described below, polar groups in the fluorine-containing ether compound are each independently capable of getting involved in bonding with active points on the protective layer for reasons <3> to <5> to be described below, and the coating state on the protective layer is likely to be uniform for a reason <6> to be described below. Due to these facts, it is estimated that the fluorine-containing ether compound represented by the formula (1) enables the formation of a lubricating layer having favorable adhesion to the protective layer and enables the formation of a lubricating layer having favorable chemical substance resistance and wear resistance and a high pickup-curbing effect.

<1> An amide bond portion (C(═O)N) that is included in a group represented by a formula (2-1) or a formula (2-2) that is included in the fluorine-containing ether compound represented by the formula (1) bonds to an active point on a protective layer to improve the adhesion of a lubricating layer to the protective layer. The amide bond portion that is included in the group represented by the formula (2-1) or the formula (2-2) that is disposed in at least one terminal bonds to an oxygen atom of —R²—CH₂—O—(R² is a perfluoropolyether chain) through a divalent linking group including no polar groups and optionally including an ether oxygen atom (X¹ in the formula (2-1) or X² in the formula (2-2)). Therefore, unlike, for example, a fluorine-containing ether compound having a divalent linking group including a polar group between the oxygen atom of —R²—CH₂—O— and the amide bond portion, the adhesion of a terminal group portion to the protective layer does not become too strong. Therefore, in a lubricating layer containing the fluorine-containing ether compound represented by the formula (1), deterioration of wear resistance due to the adhesion to the protective layer being too strong is curbed.

<2> In the fluorine-containing ether compound represented by the formula (1), since X¹ in the formula (2-1) and X² in the formula (2-2) do not include a polar group, the surface free energy of the entire molecule becomes low compared with that of, for example, a fluorine-containing ether compound having a divalent linking group including a polar group between the oxygen atom of —R²—CH₂—O— and the amide bond portion. Therefore, according to the fluorine-containing ether compound represented by the formula (1), it is possible to prevent a lubricating layer containing this compound from easily allowing attachment of a chemical substance thereto due to the surface free energy of the entire compound being too high. Therefore, the fluorine-containing ether compound represented by the formula (1) is capable of curbing deterioration of the chemical substance resistance and pickup resistance of the lubricating layer attributed to the surface free energy of the entire compound being too high.

<3> The oxygen atom of —R²—CH₂—O— and the amide bond portion (C(═O)N) that is included in the group represented by the formula (2-1) or the formula (2-2) bond to each other through a divalent linking group including no polar groups, optionally including an ether oxygen atom, and having 1 to 12 carbon atoms (X¹ in the formula (2-1) or X² in the formula (2-2)). This imparts appropriate flexibility to the group represented by the formula (2-1) or the formula (2-2) having an amide bond portion that is not easy to freely rotate. Therefore, in the fluorine-containing ether compound represented by the formula (1), the mobility (degree of freedom) of the amide bond portion is high compared with that in, for example, a fluorine-containing ether compound in which a perfluoropolyether chain represented by R² and the amide bond portion that is included in the group represented by the formula (2-1) or the formula (2-2) are directly bonded to each other. Therefore, in the fluorine-containing ether compound represented by the formula (1), the amide bond portion has a favorable affinity to an active point on the protective layer and easily bonds to the active point on the protective layer.

<4> Since X¹ in the formula (2-1) and X² in the formula (2-2) are divalent linking groups including no polar groups, optionally including an ether oxygen atom, and having 1 to 12 carbon atoms, the amide bond portion that is included in the group represented by the formula (2-1) or the formula (2-2) is capable of interacting with an active points on the protective layer without being affected by the bulkiness of the perfluoropolyether chain represented by R². Therefore, the amide bond portion that is included in the group represented by the formula (2-1) or the formula (2-2) has a favorable affinity to an active point on the protective layer and easily bonds to the active point on the protective layer.

<5> In the fluorine-containing ether compound represented by the formula (1), the amide bond portion that is included in the group represented by the formula (2-1) or the formula (2-2) that is disposed in at least one terminal is not easy to freely rotate. Therefore, even in a case where Y¹ and Z¹ in the formula (2-1) or Y² and Z² in the formula (2-2) include polar groups, the polar groups that are included in Y¹ and Z¹ in the formula (2-1) or in Y² and Z² in the formula (2-2) are less likely to be inhibited by the amide bond portion that is included in the group represented by the formula (2-1) or the formula (2-2) from bonding to an active point on the protective layer.

In addition, in the fluorine-containing ether compound represented by the formula (1), the perfluoropolyether chain represented by R² is disposed between R¹ and R⁴. Therefore, the distance between R¹ and R⁴ is appropriate. From this fact, the polar group that is included in R¹ and the polar group that is included in R⁴ do not inhibit each other from bonding to the active point on the protective layer, and the polar groups that are included in R¹ and R⁴ are less likely to be inhibited from bonding to the active point on the protective layer.

In addition, in the fluorine-containing ether compound represented by the formula (1), in a case where n is 1 or 2, a divalent linking group (R³) having a hydroxyl group is disposed between a plurality of perfluoropolyether chains represented by R². The perfluoropolyether chains represented by R² are disposed between the hydroxyl group that is included in R³ and R¹ and between the hydroxyl group and R⁴, respectively. Therefore, the distances of the hydroxyl groups that are included in R³ from R¹ and R⁴ are appropriate. From this fact, the hydroxyl groups that are included in R³ are less likely to be inhibited from bonding to the active point on the protective layer by R¹ and R⁴.

As described above, all of the polar groups that are included in the fluorine-containing ether compound represented by the formula (1) are less likely to inhibit the bonding of other polar groups to the active points on the protective layer and are less likely to be inhibited by other polar groups from bonding to the active points on the protective layer. Therefore, the polar groups that are included in the fluorine-containing ether compound represented by the formula (1) each independently exhibit a favorable interaction with the protective layer and are each independently capable of getting involved in bonding to a large number of active points present on the protective layer.

As a result, in a lubricating layer containing the fluorine-containing ether compound represented by the formula (1), polar groups that do not bond to the active points on the protective layer are less likely to be generated, the number of polar groups that do not get involved in bonding to the active points on the protective layer is curbed, and interactions between the polar groups in the molecule and/or between the molecules can be curbed.

<6> In the group represented by the formula (2-1) or the formula (2-2) that is included in the fluorine-containing ether compound represented by the formula (1), the bond of a carbon atom adjacent to a carbonyl carbon atom or nitrogen atom that configures the amide bond is not easy to freely rotate. Therefore, when the amide bond portion that is included in the group represented by the formula (2-1) or the formula (2-2) bonds to an active point on the protective layer, the perfluoropolyether chain represented by R² disposed adjacent to the group represented by the formula (2-1) or the formula (2-2) is attracted to the protective layer side and is less likely to float from the protective layer. Furthermore, the distances of the group represented by the formula (2-1) or the formula (2-2) from other polar groups that are included in the fluorine-containing ether compound are appropriate. Therefore, the amide bond portion that is included in the group represented by the formula (2-1) or the formula (2-2) is less likely to aggregate with other polar groups that are included in the fluorine-containing ether compound. From these facts, the fluorine-containing ether compound represented by the formula (1) spreads on the protective layer in a uniform coating state, is capable of forming a strong bond to the active point on the protective layer, and is capable of forming a lubricating layer having excellent adhesion to the protective layer and favorable smoothness in a uniform coating state on the protective layer.

Furthermore, the present inventors confirmed that a lubricating layer having favorable chemical substance resistance and wear resistance and having a high pickup-curbing effect can be formed by forming a lubricating layer on a protective layer of a magnetic recording medium using a lubricant containing the above-described fluorine-containing ether compound and came to an idea of the present invention.

Hereinafter, a fluorine-containing ether compound, a lubricant for a magnetic recording medium, and a magnetic recording medium of the present invention will be described in detail. The present invention is not limited only to embodiments to be described below. For the present invention, addition, omission, substitution, or change is possible without departing from the gist of the present invention regarding numbers, amounts, rates, compositions, kinds, positions, materials, configurations, and the like. “Polar group” in the present specification does not include halogeno groups (—F, —Cl, —Br, and the like) and an ether bond (—O—).

### Fluorine-Containing Ether Compound

The fluorine-containing ether compound of the present embodiment is represented by the following formula (1).

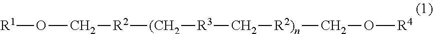

(in the formula (1), R¹ is represented by a formula (2-1) or a formula (2-2). R² is a perfluoropolyether chain. R⁴ is an organic group having at least one polar group and having 1 to 50 carbon atoms. R³ is a divalent linking group having at least one hydroxyl group. n is an integer of 0 to 2. In a case where n is 1 or 2, a part or all of a plurality of R²'s may be identical to each other or may be different from each other. In a case where n is 2, two R³'s may be identical to each other or may be different from each other.)

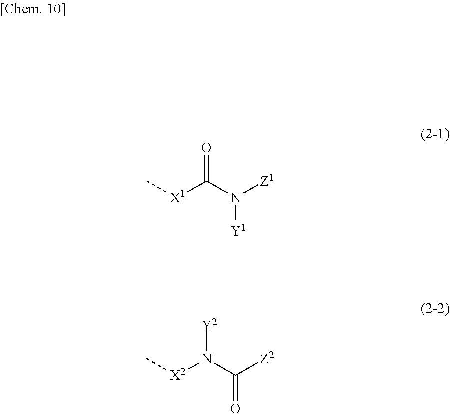

(in the formula (2-1), X¹ is a divalent linking group including no polar groups, optionally including an ether oxygen atom, and having 1 to 12 carbon atoms. Y¹ and Z¹ are each independently an organic group optionally including at least one of a polar group and an ether oxygen atom and having 1 to 30 carbon atoms or a hydrogen atom. Y¹ and Z¹ may bond to each other to form a ring.)

(in the formula (2-2), X² is a divalent linking group including no polar groups, optionally including an ether oxygen atom, and having 1 to 12 carbon atoms. Y² is an organic group optionally including at least one of a polar group and an ether oxygen atom and having 1 to 30 carbon atoms or a hydrogen atom. Ze is an organic group optionally including at least one of a polar group and an ether oxygen atom and having 1 to 30 carbon atoms. Y² and Z² may bond to each other to form a ring.)

In the fluorine-containing ether compound of the present embodiment represented by the formula (1), n indicating the number of (CH₂—R³—CH₂—R²) in the formula (1) is an integer of 0 to 2. Therefore, the fluorine-containing ether compound of the present embodiment has (n+1) perfluoropolyether chains represented by R².

In the case of having only one perfluoropolyether chain (hereinafter referred to as “PFPE chain” in some cases) (in other words, a case where n in the formula (1) is 0), the fluorine-containing ether compound of the present embodiment has a structure in which a methylene group and an ether oxygen atom bond to both sides of the PFPE chain represented by R², and the group represented by the formula (2-1) or the formula (2-2) bonds to at least one terminal.

In the case of including a plurality of the PFPE chains (in other words, a case where n in the formula (1) is 1 or 2), the fluorine-containing ether compound of the present embodiment has a skeleton in which a divalent linking group represented by R³ interposed between two methylene groups is disposed between the PFPE chains represented by R². In this case, the fluorine-containing ether compound has a structure in which a methylene group and an ether oxygen atom bond to both sides of the above-described skeleton, and the group represented by the formula (2-1) or the formula (2-2) bonds to at least one terminal thereof.

In the fluorine-containing ether compound of the present embodiment, n in the formula (1) is an integer of 0 to 2.

In a case where n is 0, a group having an amide bond bonds to at least one terminal of the PFPE chain represented by R² as the center. Therefore, the fluorine-containing ether compound easily spreads on the protective layer uniformly, and makes it easy to obtain a lubricating layer having a uniform film thickness, which is preferable.

In a case where n is 1 or 2, a methylene group and an ether oxygen atom are disposed in this order at both ends of the PFPE chains represented by R² linked by the divalent linking group represented by R³, and an organic group having an amide bond bonds to at least one terminal thereof. Additionally, since the divalent linking group represented by R³ has at least one hydroxyl group, the adhesion to the active point on the protective layer becomes favorable. Therefore, the fluorine-containing ether compound easily spreads on the protective layer uniformly, and makes it easy to obtain a lubricating layer having a uniform film thickness, which is preferable.

### Perfluoropolyether Chain Indicated by R²

In the fluorine-containing ether compound represented by the formula (1), (n+1) R²'s are each independently a PFPE chain. The PFPE chain indicated by R² coats the surface of a protective layer and imparts lubricity to a lubricating layer to reduce a friction force between a magnetic head and the protective layer in a case where a lubricant containing the fluorine-containing ether compound of the present embodiment is applied onto the protective layer to form the lubricating layer. The PFPE chain indicated by R² is selected as appropriate depending on performances or the like that are required for the lubricant containing the fluorine-containing ether compound.

In the fluorine-containing ether compound represented by the formula (1), (n+1) R²'s may be partially or fully identical to each other or may be each different from each other. (n+1) R²'s are preferably all identical to each other. This is because the coating state of the fluorine-containing ether compound on the protective layer becomes more uniform, and a lubricating layer having more favorable adhesion can be obtained.

The fact that, among (n+1) R²'s, two or more R²'s are identical to each other means that, among (n+1) R²'s, two or more R²'s in which the structures of the repeating units of the PFPE chains are identical to each other are included. The identical R²'s include R²'s in which the structures of the repeating units are identical to each other, but the average degrees of polymerization are different from each other.

Examples of the PFPE chain indicated by R² include PFPE chains composed of a polymer or copolymer of a perfluoroalkylene oxide. Examples of the perfluoroalkylene oxide include perfluoromethylene oxide, perfluoroethylene oxide, perfluoro-n-propylene oxide, perfluoroisopropylene oxide, perfluorobutylene oxide, and the like.

(n+1) R²'s in the formula (1) are each independently preferably a PFPE chain represented by the following formula (6) that is derived from the polymer or copolymer of the perfluoroalkylene oxide.

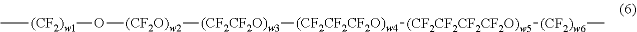

(in the formula (6), w2, w3, w4, and w5 indicate the average degrees of polymerization and each independently represent 0 to 20. Here, w2, w3, w4, and w5 do not all become 0 at the same time. w1 and w6 are the average values representing the numbers of CF₂ and each independently represent 1 to 3. An array order of (CF₂O), (CF₂CF₂O), (CF₂CF₂CF₂O), and (CF₂CF₂CF₂CF₂O), which are repeating units in the formula (6), is not particularly limited.)

In the formula (6), w2, w3, w4, and w5 indicate the average degrees of polymerization, each independently represent 0 to 20, and are preferably 0 to 15 and more preferably 0 to 10.

In the formula (6), w1 and w6 are the average values indicating the numbers of CF₂ and each independently represent 1 to 3. w1 and w6 are determined depending on the structure or the like of a repeating unit that is disposed at an end portion of a chain structure in the PFPE chain represented by the formula (6).

(CF₂O), (CF₂CF₂O), (CF₂CF₂CF₂O), and (CF₂CF₂CF₂CF₂O) in the formula (6) are repeating units. The array order of the repeating units in the formula (6) is not particularly limited. In addition, the number of the kinds of the repeating units in the formula (6) is also not particularly limited.

(n+1) R²'s in the formula (1) are each independently preferably any one selected from PFPE chains represented by the following formulae (6-1) to (6-4).

When (n+1) R²'s are each independently any one selected from the PFPE chains represented by the formulae (6-1) to (6-4), it becomes possible to obtain a lubricating layer having favorable lubricity from the fluorine-containing ether compound. In addition, in a case where (n+1) R²'s are each independently any one selected from the PFPE chains represented by the formulae (6-1) to (6-4), the proportion of the number of oxygen atoms (the number of ether bonds (—O—)) in the number of carbon atoms in the PFPE chain is appropriate. Therefore, the fluorine-containing ether compound has an appropriate hardness. Therefore, the fluorine-containing ether compound applied onto the protective layer is less likely to aggregate on the protective layer, and it is possible to form a lubricating layer of thinner thickness with a sufficient coating rate. In addition, the fluorine-containing ether compound is appropriately flexible and is thereby capable of forming a lubricating layer having more favorable chemical substance resistance.

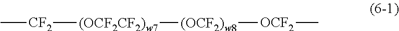

(in the formula (6-1), w7 and w8 indicate average degrees of polymerization, w7 represents 1 to 20, and w8 represents 0 to 20.)

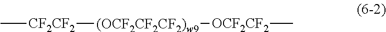

(in the formula (6-2), w9 indicates an average degree of polymerization and represents 1 to 15.)

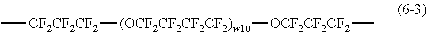

(in the formula (6-3), w10 indicates an average degree of polymerization and represents 1 to 10.)

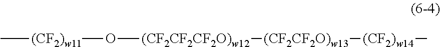

(in the formula (6-4), w12 and w13 indicate average degrees of polymerization and each independently represent 1 to 20. w11 and w14 are average values representing the numbers of CF₂ and each independently represent 1 to 2.)

In the formula (6-1), the array order of (OCF₂CF₂) and (OCF₂), which are repeating units, is not particularly limited. In the formula (6-1), the number w7 of (OCF₂CF₂)'s and the number w8 of (OCF₂)'s may be identical to each other or different from each other. The PFPE chain represented by the formula (6-1) may be a polymer of (OCF₂CF₂). In addition, the PFPE chain represented by the formula (6-1) may be any of a random copolymer, a block copolymer, and an alternating copolymer that are all composed of (OCF₂CF₂) and (OCF₂).

In the formulae (6-1) to (6-3), since w7 is 1 to 20, w8 is 0 to 20, w9 is 1 to 15, and w10 is 1 to 10, w7, w8, w9, and w10 indicating the average degrees of polymerization, it becomes possible to obtain a lubricating layer having favorable lubricity from the fluorine-containing ether compound. In addition, in the formulae (6-1) to (6-3), since w7 and w8 are 20 or less, w9 is 15 or less, and w10 is 10 or less, w7, w8, w9, and w10 indicating the average degrees of polymerization, the viscosity of the fluorine-containing ether compound does not become too high, and a lubricant containing this fluorine-containing ether compound is easy to apply, which is preferable. w7, w8, w9, and w10 indicating the average degrees of polymerization are preferably 1 to 10, more preferably 1.5 to 8, and still more preferably 2 to 7 since the fluorine-containing ether compound easily spreads on the protective layer, and it becomes easy to obtain a lubricating layer having a uniform film thickness from the fluorine-containing ether compound.

In the formula (6-4), the array order of (CF₂CF₂CF₂O) and (CF₂CF₂O), which are repeating units, is not particularly limited. In the formula (6-4), the number w12 of (CF₂CF₂CF₂O)'s and the number w13 of (CF₂CF₂O)'s, which indicate the average degrees of polymerization, may be identical to each other or different from each other. The formula (6-4) may be a PFPE chain containing any of a random copolymer, a block copolymer, and an alternating copolymer that are all composed of monomer units (CF₂CF₂CF₂O) and (CF₂CF₂O).

In the formula (6-4), w12 and w13 indicating the average degrees of polymerization are each independently 1 to 20, preferably 1 to 15, and more preferably 1 to 10.

w11 and w14 in the formula (6-4) are the average values indicating the numbers of CF₂ and each independently represent 1 to 2. w11 and w14 are determined depending on the structure or the like of a repeating unit that is disposed at an end portion of a chain structure in the perfluoropolyether chain represented by the formula (6-4).

### Divalent Linking Group Having Hydroxyl Group Indicated by R³

In the fluorine-containing ether compound represented by the formula (1), R³ is a divalent linking group having at least one hydroxyl group. In a case where n is 1 or 2, R³ is disposed between adjacent PFPE chains represented by R². Due to this fact, R³ makes the fluorine-containing ether compound and the protective layer closely adhere to each other and forms a lubricating layer of thin thickness with a sufficient coating rate.

In a case where n, which is the number of (CH₂—R³—CH₂—R²)'s in the formula (1), is 2, two R³'s may be identical to each other or may be different from each other. When the two R³'s are identical to each other, the coating state of the fluorine-containing ether compound on the protective layer becomes more uniform, and a lubricating layer having more favorable adhesion can be formed.

In the present specification, the fact that “two R³'s are identical to each other” in a case where n is 2 means that atoms that are included in the two R³'s are symmetrically disposed with respect to R²'s disposed at the center of the fluorine-containing ether compound represented by the formula (1) among three R²'s in the fluorine-containing ether compound.

The number of hydroxyl groups that are included in the divalent linking group indicated by R³ is preferably 1 to 3 and more preferably 1. Since one or more hydroxyl groups are included in R³, the plurality of R²'s linked through R³ are less likely to float from the protective layer, and the adhesion between the lubricating layer and the protective layer is less likely to deteriorate. When the number of the hydroxyl groups that are included in R³ is 3 or less, hydroxyl groups that do not get involved in bonding to the active point on the protective layer are far less likely to be generated, which is preferable.

The divalent linking group indicated by R³ preferably has oxygen atoms disposed at both end portions thereof. The oxygen atoms disposed at both end portions of the linking group form methylene groups (—CH₂—) and ether bonds (—O—) disposed on both sides of R³. These two ether bonds impart appropriate flexibility to the fluorine-containing ether compound represented by the formula (1) and increase the affinity of the hydroxyl group in the divalent linking group represented by R³ to the protective layer.

The divalent linking group indicated by R³ is preferably a group in which a group in which a hydrogen atom bonding to at least one carbon atom of an alkylene group having 3 to 7 carbon atoms has been substituted with a hydroxyl group is disposed between two oxygen atoms disposed at both ends. The alkylene group having 3 to 7 carbon atoms is preferably an alkylene group having 3 to 5 carbon atoms. The alkylene group having 3 to 7 carbon atoms preferably has a linear structure. Particularly, R³ is a group in which oxygen atoms are disposed at both ends of a linear alkylene group having 3 to 7 carbon atoms, respectively, and a hydroxyl group bonds to a carbon atom that is disposed near the center of the linear alkylene group. This is because the coating state of the fluorine-containing ether compound on the protective layer becomes more uniform and a lubricating layer has more favorable adhesion.

Specifically, R³ is preferably represented by the following formula (4). When R³ is the formula (4), the synthesis of the fluorine-containing ether compound represented by the formula (1) is easy, which is preferable.

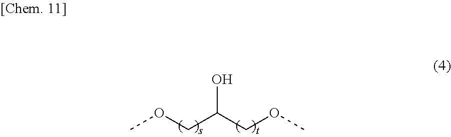

(in the formula (4), s is an integer of 1 to 3, and t is an integer of 1 to 3. The oxygen atom at the left terminal in the formula (4) bonds to —CH₂— on the R¹ side, and the oxygen atom at the right terminal bonds to —CH₂— on the R⁴ side.)

In the formula (4), s is an integer of 1 to 3, preferably an integer of 1 or 2, and more preferably 1. This is because the coating state of the fluorine-containing ether compound on the protective layer becomes more uniform and a lubricating layer has more favorable adhesion.

In the formula (4), t is an integer of 1 to 3, preferably an integer of 1 or 2, and more preferably 1. This is because the coating state of the fluorine-containing ether compound on the protective layer becomes more uniform and a lubricating layer has more favorable adhesion.

In the formula (4), in a case where s and/or t is an integer of 2 or more, the distance between the hydroxyl group in the formula (4) and the PFPE chain becomes long compared to that in a case where s and t are 1. Therefore, the influence of the bulkiness of the PFPE chain represented by R² on the hydroxyl group in R³ becomes smaller, and a lubricating layer having high adhesion to the protective layer can be formed.

### Terminal Group Indicated by R¹

In the formula (1), R¹ is a group having an amide bond and is represented by the formula (2-1) or (2-2). In the fluorine-containing ether compound of the present embodiment, the amide bond portion that is included in the terminal group indicated by R¹ exhibits a strong interaction with the active point on the protective layer.

The terminal group indicated by R¹ is selected as appropriate depending on performances or the like that are required for the lubricant containing the fluorine-containing ether compound.

X¹ in Formula (2-1) and X² in Formula (2-2)

X¹ in the formula (2-1) and X² in the formula (2-2) are divalent linking groups including no polar groups, optionally including an ether oxygen atom, and having 1 to 12 carbon atoms.

Since X¹ and X² are divalent linking groups having 1 or more carbon atoms, the distance between the PFPE chain indicated by R² and the amide bond portion (C(═O)N) that is included in the formula (2-1) and the formula (2-2) is not too close. Therefore, the amide bond portion that is included in the formula (2-1) or the formula (2-2) is not affected by the bulkiness of the PFPE chain and is capable of closely adhering to the protective layer.

In addition, X¹ and X² are divalent linking groups having 12 or less carbon atoms, and the distances between the PFPE chain indicated by R² and the amide bond portion that is included in the formula (2-1) and the formula (2-2) are thus not too far. Therefore, it is possible to effectively curb the PFPE chain floating from the protective layer due to the interaction between the amide bond portion that is included in the formula (2-1) and the formula (2-2) and the protective layer. Since the distance between the amide bond portion that is included in the formula (2-1) and the formula (2-2) and the PFPE chain indicated by R² becomes more appropriate, the numbers of carbon atoms that are included in the divalent linking groups indicated by X¹ and X² are preferably 1 to 10 and more preferably 1 to 6.

X¹ in the formula (2-1) and X² in the formula (2-2) each preferably have a linear structure and preferably do not include any ring structures. This is because in the case of having a linear structure, X¹ and X² are less likely to inhibit the interaction between the amide bond portion that is included in the formula (2-1) and the formula (2-2) and the protective layer.

X¹ in the formula (2-1) and X² in the formula (2-2) include no polar groups. Therefore, as described in the <1>, in the fluorine-containing ether compound represented by the formula (1), the adhesion of the group represented by the formula (2-1) or (2-2) to the protective layer does not become too strong unlike in cases where X¹ or X² includes a polar group. Therefore, it is possible to curb deterioration of the wear resistance due to the adhesion to the protective layer being too strong. In addition, as described in the <2>, since X¹ or X² does not include polar groups, it is possible to curb deterioration of the chemical substance resistance and pickup resistance of the lubricating layer containing this fluorine-containing ether compound due to the surface free energy of the entire compound being too high.

The divalent linking groups represented by X¹ in the formula (2-1) and X² in the formula (2-2) may include one or more ether oxygen atoms (—O—). In a case where the divalent linking groups each include an ether oxygen atom, the formula (2-1) or formula (2-2) has appropriate flexibility. Therefore, the lubricating layer containing the fluorine-containing ether compound indicated by the formula (1) has superior adhesion to the protective layer. In a case where the divalent linking groups represented by X¹ and X² each include ether oxygen atoms, the number of the ether oxygen atoms is preferably 1 or 2 and more preferably 1.

In a case where the divalent linking groups represented by X¹ in the formula (2-1) and X² in the formula (2-2) each have a plurality of ether oxygen atoms, adjacent ether oxygen atoms preferably bond to each other through a linking group to which two or more carbon atoms are linked. This is because the distance between the adjacent ether oxygen atoms becomes appropriate, and the fluorine-containing ether compound is less likely to aggregate.

X¹ in the formula (2-1) and X² in the formula (2-2) are each independently preferably a divalent linking group represented by the following formula (3).

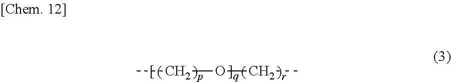

(The left terminal in the formula (3) is a side bonding to an oxygen atom of —R²—CH₂—O—, and the right terminal is a side bonding to a carbonyl carbon atom or nitrogen atom that configures an amide bond.)

In a case where the divalent linking groups represented by X¹ and X² are each a group represented by the formula (3), the distance between the perfluoropolyether chain represented by R² and the amide bond portion that is included in the formula (2-1) or the formula (2-2) becomes neither too close nor too far, but appropriate. Therefore, the amide bond portion is capable of strongly interacting with the active point on the protective layer.

In the formula (3), q is an integer of 0 to 2. In a case where q is 2, p in each [—(CH₂)ₚ—O—] may be identical to each other or different from each other. In a case where q in the formula (3) is 1 or 2, X¹ in the formula (2-1) and X² in the formula (2-2) each include an ether bond, whereby the fluorine-containing ether compound becomes appropriately flexible, which is preferable. In addition, since q is 2 or less, the number of the ether oxygen atoms that are included in X¹ or X² is not too large. Therefore, in a case where X¹ or X² is a linking group represented by the formula (3), the fluorine-containing ether compound applied onto the protective layer is less likely to aggregate on the protective layer, and it is possible to form a lubricating layer of thinner thickness with a sufficient coating rate.

In the formula (3), p is an integer of 2 to 4. When p is 2 or more, in a case where q is 1 or 2, the distance between the adjacent perfluoropolyether chain indicated by R² and the amide bond that is included in the formula (2-1) or the formula (2-2) becomes more appropriate. In addition, when p is 2 or more, two or more methylene groups are disposed between the oxygen atom of —R²—CH₂—O— and the ether oxygen atom in the formula (3) and between the ether oxygen atoms in the formula (3). Therefore, the fluorine-containing ether compound is less likely to aggregate. In addition, when p is 4 or less, even when q is 1 or 2, the linking group represented by the formula (3) does not become too long, and the distance between the adjacent perfluoropolyether chain represented by R² and the amide bond that is included in the formula (2-1) or the formula (2-2) becomes appropriate. p in the formula (3) is preferably 2 or 3. When p is 2 or 3, the molecular weight of the formula (2-1) or the formula (2-2) is large, whereby it is possible to curb the surface free energy of the entire molecule becoming too large due to a decrease in the proportion of fluorine atoms in the fluorine-containing ether compound molecule.

In the formula (3), r is an integer of 1 to 4. When r is an integer of 1 to 4, the distance between the adjacent perfluoropolyether chain represented by R² and the amide bond that is included in the formula (2-1) or the formula (2-2) becomes appropriate regardless of the value of q. Furthermore, when r in the formula (3) is an integer of 1 to 4, the molecular weight of the formula (2-1) or the formula (2-2) is large, whereby it is possible to curb the surface free energy of the entire molecule becoming too large due to a decrease in the proportion of fluorine atoms in the fluorine-containing ether compound molecule. r is preferably 1 to 3 and more preferably 1 or 2.

In the formula (3), in a case where q is 0, r is preferably 1 or 2. In this case, since the number of methylenes that are included in X¹ in the formula (2-1) and X² in the formula (2-2) is not too large, even when X¹ and X² do not include ether oxygen atoms, the divalent linking group portion does not lack flexibility, and it is possible to curb deterioration of the affinity of the amide bond portion to the protective layer.

Y¹ and Z¹ in Formula (2-1) and Y² and Z² in Formula (2-2)

Y¹ and Z¹ in the formula (2-1) and Y² in the formula (2-2) are each independently an organic group optionally including at least one of a polar group and an ether oxygen atom and having 1 to 30 carbon atoms or a hydrogen atom. 72 in the formula (2-2) is an organic group optionally including at least one of a polar group and an ether oxygen atom and having 1 to 30 carbon atoms.

In a case where Y¹ and Z¹ in the formula (2-1) and Y² and Z² in the formula (2-2) are the above-described organic groups, the organic groups may each include one or more polar groups. Examples of the polar group include a hydroxyl group (—OH), an amino group (—NR⁵R⁶; R⁵ and R⁶ are each independently a hydrogen atom or an organic group), a carboxy group (—COOH), a formyl group (—(C═O)H), a carbonyl group (—CO—), a sulfo group (—SO₃H), a cyano groups (—CN), and the like. In a case where the organic group includes a hydroxyl group as the polar group, the hydroxyl group may be a primary hydroxyl group or may be a secondary hydroxyl group or a tertiary hydroxyl group. In a case where the organic groups include the polar group, since a polar group that does not get involved in the bond with the active point present on the protective layer is far less likely to be generated, the number of the polar groups is preferably 1 or 2 and more preferably 1.

In a case where Y¹, Z¹, Y², and Z² are the organic groups, the organic groups may each include one or more ether oxygen atoms. In a case where the organic groups include an ether oxygen atom, the organic groups represented by Y¹, Z¹, Y², and Z² become appropriately flexible, and the amide bond portion becomes easy to adhere to the protective layer. Therefore, it becomes possible to form a lubricating layer having superior adhesion to the protective layer from the fluorine-containing ether compound. In a case where the organic groups represented by Y¹, Z¹, Y², and Z² include the ether oxygen atom, the number of the ether oxygen atoms is preferably 1 or 2 and more preferably 1 since the fluorine-containing ether compound is less likely to aggregate.

Y¹ and Z¹ in the formula (2-1) and Y² and Z² in the formula (2-2) are each preferably an organic group including no polar groups and having 1 to 15 carbon atoms or a hydrogen atom, more preferably an alkyl group having 1 to 12 carbon atoms or a hydrogen atom, and still more preferably any group of a methyl group, an ethyl group, a propyl group, and a hydrogen atom. Here, Z² is not a hydrogen atom.

Y¹ and Z¹ in the formula (2-1) and Y² and Z² in the formula (2-2) may each bond to each other to form a ring. Examples of the ring that Y¹ and Z¹ and Y² and Z² each bond to each other to form include rings having a structure in which -Y¹-Z¹- or -Y²-Z²- is formed by a linking group composed of one or more methylene groups to which a polar group may bond and an ether oxygen atom, rings having a structure in which -Y¹-Z¹- or -Y²-Z²- is formed by linking a plurality of methylene groups to which a polar group may bond, and the like. The rings that may be formed by Y¹ and Z¹ and Y² and Z² each bonding to each other are preferably five to seven-membered rings and more preferably five-membered rings or six-membered rings.

Y¹ and Z¹ in the formula (2-1) (or Y² and Z² in the formula (2-2)) may be identical to each other or may be different from each other.

In the formula (2-1), at least one of Y¹ and Z¹ is preferably a hydrogen atom. This is because the affinity of the amide bond portion to the protective layer becomes excellent in the fluorine-containing ether compound including the formula (2-1) compared with that in a case where both Y¹ and Z¹ are organic groups having 1 to 30 carbon atoms.

In addition, in the formula (2-2), Y² is preferably a hydrogen atom. This is because the affinity of the amide bond portion to the protective layer becomes excellent in the fluorine-containing ether compound including the formula (2-2) compared with that in a case where Y² is an organic group having 1 to 30 carbon atoms.

Examples of the structure of the formula (2-1) include the following formulae (2-1-A) to (2-1-I), but are not limited thereto.

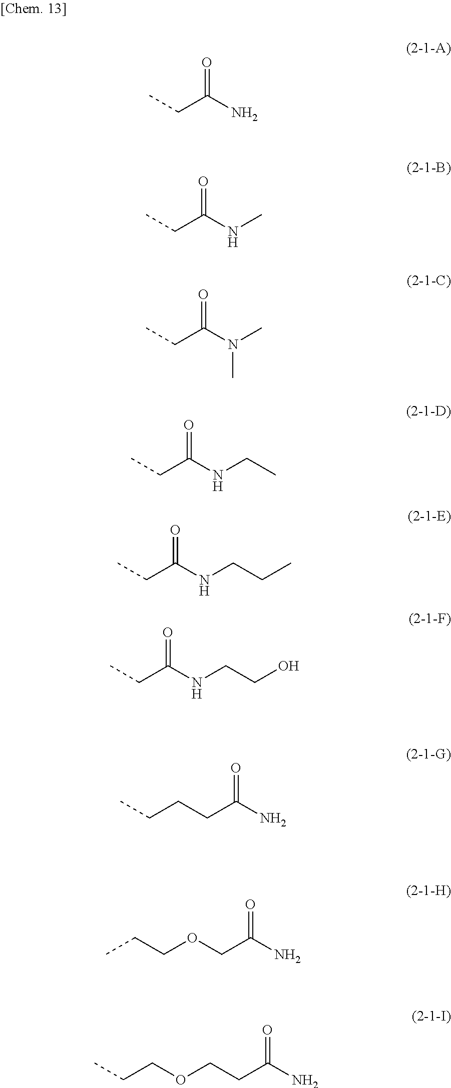

Examples of the structure of the formula (2-2) include the following formulae (2-2-A) to (2-2-I), but are not limited thereto.

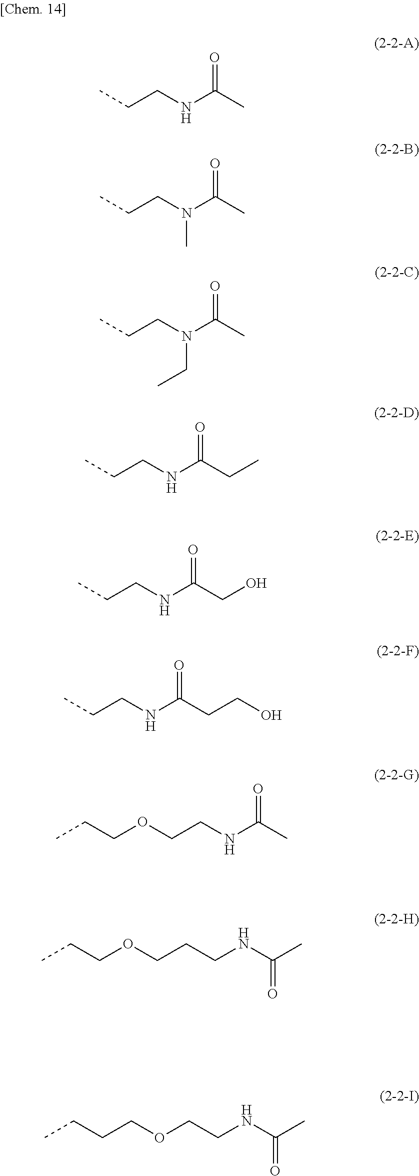

### Terminal Group Indicated by R⁴

In the fluorine-containing ether compound represented by the formula (1), the terminal group indicated by R⁴ is an organic group having at least one polar group and having 1 to 50 carbon atoms. The terminal group indicated by R⁴ is preferably an organic group having 1 to 30 carbon atoms and more preferably an organic group having 2 to 20 carbon atoms. When the number of the carbon atoms is within the above-described range, the proportion of the number of the carbon atoms to the number of the polar groups becomes appropriate, and the polarity of the molecule becomes appropriate in the fluorine-containing ether compound.

Examples of the polar group that the terminal group indicated by R⁴ has include a hydroxyl group (—OH), an amino group (—NR⁵R⁶; R⁵ and R⁶ are each independently a hydrogen atom or an organic group), a carboxy group (—COOH), a formyl group (—(C═O)H), a carbonyl group (—CO—), a sulfo group (—SO₃H), a cyano group (—CN), and the like.

In a case where the terminal group indicated by R⁴ has a polar group including a carbon atom (for example, a carboxy group, a formyl group, a carbonyl group, or a cyano group), carbon atoms that are included in the polar group are included in the number of carbon atoms in the terminal group indicated by R⁴.

The number of the polar groups that the terminal group indicated by R⁴ has is preferably 1 to 3, more preferably 2 or 3, and most preferably 2 since it becomes possible to form a lubricating layer having more favorable adhesion to the protective layer from the fluorine-containing ether compound. When the number of the polar groups is 3 or less, in a magnetic recording medium having a lubricating layer containing the fluorine-containing ether compound, it is possible to prevent aggregation of the fluorine-containing ether compound due to an excessively large number of the polar groups that are included in the fluorine-containing ether compound and the deterioration of smoothness. In addition, when the number of the polar groups is 3 or less, it is possible to curb deterioration of the wear resistance due to the adhesion to the protective layer being too strong.

In a case where R⁴ includes two or more polar groups, it is preferable that the two or more polar groups each bond to a different carbon atom and one or more carbon atoms are included between carbon atoms to which adjacent polar groups bond. In this case, the carbon atoms bond to each other with the adjacent polar groups in an appropriate interatomic distance compared with a case where the carbon atoms to which the adjacent polar groups bond directly bond to each other. Therefore, the plurality of polar groups that R⁴ has are all oriented to be capable of closely adhering onto the protective layer. Due to this fact, the plurality of polar groups that R⁴ has are less likely to aggregate, and the bond with the active point on the protective layer can be easily formed.

The terminal group indicated by R⁴ has may be an organic group having 1 to 3 polar groups and further having a carbon-carbon unsaturated bond portion. In a case where the terminal group indicated by R⁴ has the carbon-carbon unsaturated bond portion, the terminal group is preferably an organic group having at least one selected from the group consisting of an aromatic hydrocarbon group, an unsaturated heterocyclic group, an alkenyl group, and an alkynyl group.

Examples of the aromatic hydrocarbon group include a phenyl group, a methoxyphenyl group, a fluorinated phenyl group, a naphthyl group, a methoxynaphthyl group, and the like. The aromatic hydrocarbon group also includes groups having a substituent, such as a methoxy group or a fluoro group, bonding to an aromatic hydrocarbon.

Examples of the unsaturated heterocyclic group include a pyrrolyl group, a pyrazolyl group, a methylpyrazolyl group, an imidazolyl group, a furyl group, a furfuryl group, an oxazolyl group, an isoxazolyl group, a thienyl group, a thiazolyl group, an isothiazolyl group, a pyridyl group, a pyrimidinyl group, a pyridazinyl group, a pyrazinyl group, an indolinyl group, a benzofuranyl group, a benzothienyl group, a benzimidazolyl group, a benzoxazolyl group, a benzothiazolyl group, a benzopyrazolyl group, a benzisoxazolyl group, a benzisothiazolyl group, a quinolyl group, an isoquinolyl group, a quinazolinyl group, a quinoxalinyl group, a phthalazinyl group, a cinnolinyl group, and the like. The unsaturated heterocyclic group also include groups having a substituent, such as a methyl group, bonding to an unsaturated heterocycle.

Examples of the alkenyl group include a vinyl group, an allyl group, a butenyl group, a pentenyl group, a hexenyl group, and the like.

Examples of the alkynyl group include a 1-propynyl group, a propargyl group, a butynyl group, a pentynyl group, a hexynyl group, and the like.

In a case where the terminal group indicated by R⁴ has the carbon-carbon unsaturated bond portion, a lubricating layer containing the fluorine-containing ether compound has excellent adhesion to the protective layer, and the thickness can be thinned, which is preferable. The reasons therefor will be described below.

In a large number of the active points present on the protective layer, there is a locally charged portion and a portion where the distribution of charges spreads. The amide bond portion that is included in R¹ in the formula (1), the hydroxyl group in a case where R¹ has a hydroxyl group, the hydroxyl group that is included in R³ in a case where n is 1 or more, the hydroxyl group in a case where R⁴ has a hydroxyl group, (which will be collectively referred to as “the amide bond portion and the hydroxyl group”) and the carbon-carbon unsaturated bond portion that is included in the terminal group indicated by R⁴ are adsorbed to different portions on the protective layer.

Specifically, the amide bond portion and the hydroxyl group in the formula (1) exhibit an adsorption capability since the hydrogen atom and the carbonyl group interact with the locally charged portion on the protective layer. On the other hand, the carbon-carbon unsaturated bond portion that is included in the terminal group indicated by R⁴ has a nonlocal charge and thus interact with the portion where the distribution of charges spreads on the protective layer, thereby exhibiting an adsorption capability.

Therefore, the amide bond portion and the hydroxyl group in the formula (1) and the carbon-carbon unsaturated bond portion that is included in the terminal group indicated by R⁴ are each independently capable of interacting with the active points on the protective layer. As a result, a lubricating layer containing the fluorine-containing ether compound in which the terminal group indicated by R⁴ has the carbon-carbon unsaturated bond portion has superior adhesion to the protective layer and high pickup resistance.

R⁴ may be a terminal group represented by the formula (2-1) or the formula (2-2). In this case, R⁴ includes an amide bond portion as a polar group. In this case, both R¹ and R⁴ include the amide bond portion, and the amide bond portions that are included in R¹ and R⁴ are each capable of forming a strong interaction with the protective layer.

Examples of a preferable structure in a case where R⁴ is the terminal group represented by the formula (2-1) or the formula (2-2) is the same as those described regarding the terminal group indicated by R¹.

In a case where R⁴ is the terminal group represented by the formula (2-1) or the formula (2-2), R¹ and R⁴ may be identical to or different from each other but are preferably identical to each other. When R¹ and R⁴ are identical to each other, the coating state of the fluorine-containing ether compound on the protective layer becomes more uniform, and a lubricating layer having more favorable adhesion can be formed. In addition, when R¹ and R⁴ are identical to each other, the fluorine-containing ether compound can be easily produced by a small number of steps, which is preferable.

In a case where R⁴ is not the terminal group represented by the formula (2-1) or the formula (2-2), since it becomes possible to form a lubricating layer having excellent adhesion to the protective layer, among the polar groups that are included in R⁴, at least one is preferably a hydroxyl group.

In a case where R⁴ is not the terminal group represented by the formula (2-1) or the formula (2-2), it is preferable that the terminal group represented by R⁴ includes two or three polar groups and at least one of the polar groups is a terminal group that is a secondary hydroxyl group. This is because it is possible to form a lubricating layer having excellent adhesion to the protective layer, and it is easy to obtain a lubricating layer having all of the characteristics, such as the chemical substance resistance, the wear resistance, and the pickup resistance, that are as favorable as or more favorable than those in a case where R⁴ is the terminal group represented by the formula (2-1) or the formula (2-2).

In a case where the terminal group represented by R⁴ is not the terminal group represented by the formula (2-1) or the formula (2-2), the terminal group represented by R⁴ is specifically preferably any of terminal groups indicated by the following formulae (5-1) to (5-3). This is because it is possible to form a lubricating layer having favorable chemical substance resistance and wear resistance and having excellent pickup resistance.

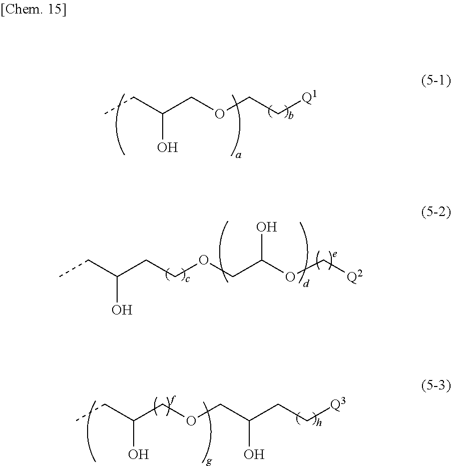

(in the formula (5-1), a is an integer of 1 or 2, and b is an integer of 0 to 3. Q¹ is an aromatic hydrocarbon group, an unsaturated heterocyclic group, an alkenyl group, an alkynyl group, or a polar group. In a case where a is 1, Q¹ is a polar group. In a case where Q¹ is an aromatic hydrocarbon group or an unsaturated heterocyclic group, an atom that configures a ring structure in Q¹ bonds to a methylene group adjacent to Q¹. In a case where Q¹ is an alkenyl group or an alkynyl group, a carbon atom that configures an unsaturated bond in Q¹ bonds to the methylene group adjacent to Q¹.)

(in the formula (5-2), c is an integer of 1 to 3, d is an integer of 0 or 1, and e is an integer of 0 to 3. Q² is an aromatic hydrocarbon group, an unsaturated heterocyclic group, an alkenyl group, an alkynyl group, or a polar group. In a case where d is 0, Q² is a polar group. In a case where Q² is an aromatic hydrocarbon group or an unsaturated heterocyclic group, an atom that configures a ring structure in Q² bonds to a methylene group adjacent to Q². In a case where Q² is an alkenyl group or an alkynyl group, a carbon atom that configures an unsaturated bond in Q² bonds to the methylene group adjacent to Q².)

(in the formula (5-3), f is an integer of 1 to 3, g is an integer of 0 or 1, and h is an integer of 0 to 3. Q³ is an aromatic hydrocarbon group, an unsaturated heterocyclic group, an alkenyl group, an alkynyl group, or a polar group. In a case where g is 0, Q³ is a polar group. In a case where Q³ is an aromatic hydrocarbon group or an unsaturated heterocyclic group, an atom that configures a ring structure in Q³ bonds to a methylene group adjacent to Q³. In a case where Q³ is an alkenyl group or an alkynyl group, a carbon atom that configures an unsaturated bond in Q³ bonds to the methylene group adjacent to Q³.)

In the formulae (5-1) to (5-3), in a case where Q¹ to Q³ are aromatic hydrocarbon groups, the atoms that configure the ring structures in Q¹ to Q³ bond to the methylene groups adjacent to Q¹ to Q³. In a case where Q¹ to Q³ are aromatic hydrocarbon group, it is possible to use the aromatic hydrocarbon groups exemplified above as Q¹ to Q³.

In the formulae (5-1) to (5-3), in a case where Q¹ to Q³ are unsaturated heterocyclic groups, the atoms that configure the ring structures in Q¹ to Q³ bond to the methylene groups adjacent to Q¹ to Q³. In a case where Q¹ to Q³ are unsaturated heterocyclic group, it is possible to use the unsaturated heterocyclic groups exemplified above as Q¹ to Q³.

In the formulae (5-1) to (5-3), in a case where Q¹ to Q³ are alkenyl groups, the carbon atoms that configure the unsaturated bonds in Q¹ to Q³ bond to the methylene groups adjacent to Q¹ to Q³. In a case where Q¹ to Q³ are alkenyl groups, examples of Q¹ to Q³ include —CH═CH₂, —CH═CHR¹¹ (R¹¹ is an organic group), —CR¹²═CHR¹³ (R¹² and R¹³ are organic groups), —CR¹⁴—CR¹⁵R¹⁶ (R¹⁴, R¹⁵, and R¹⁶ are organic groups), and the like. The organic groups represented by R¹¹ to R¹⁶ are each preferably a hydrocarbon group having 1 to 3 carbon atoms. In a case where Q¹ to Q³ in the formulae (5-1) to (5-3) are alkenyl groups, Q¹ to Q³ are preferably —CH═CH₂. —CH═CH₂ is appropriately bulky. Therefore, a lubricating layer containing the fluorine-containing ether compound having a terminal group in which Q¹ to Q³ are —CH═CH₂ is likely to form a state in which the bulkiness of the fluorine-containing ether compound on the protective layer is low and has favorable smoothness.

In the formulae (5-1) to (5-3), in a case where Q¹ to Q³ are alkynyl groups, the carbon atoms that configure the unsaturated bonds in Q¹ to Q³ bond to the methylene groups adjacent to Q¹ to Q³. In a case where Q¹ to Q³ are alkynyl groups, examples of Q¹ to Q³ include —C≡CH, —C≡CR¹⁷ (R¹⁷ is an organic group), and the like. The organic group represented by R¹⁷ is preferably a hydrocarbon group having 1 to 3 carbon atoms. In a case where Q¹ to Q³ in the formulae (5-1) to (5-3) are alkynyl groups, Q¹ to Q³ are each preferably —C≡CH since the terminal group is appropriately bulky.

In the formulae (5-1) to (5-3), in a case where Q¹ to Q³ are polar groups, it is possible to use the polar groups exemplified above as Q¹ to Q³. Among these polar groups, Q¹ to Q³ are each preferably a hydroxyl group or a cyano group. When Q¹ to Q³ are each a hydroxyl group or a cyano group, in the case of forming a lubricating layer on the protective layer using a lubricant containing these Q¹ to Q³, a more appropriate interaction is generated between the lubricating layer and the protective layer.

In a case where Q¹ to Q³ in the formulae (5-1) to (5-3) are each a polar group, a lubricating layer containing the fluorine-containing ether compound has superior adhesion to the protective layer, and the thickness can be thinned, which is preferable. The reasons therefor will be described below.

In the formulae (5-1) to (5-3), a secondary hydroxyl group and Q¹ to Q³ in the formulae (5-1) to (5-3) bond to each other through a divalent organic group optionally including an ether bond. Therefore, even when Q¹ to Q³ are each a polar group, the distance between the secondary hydroxyl group and the polar group represented by Q¹ to Q³ in the formulae (5-1) to (5-3) becomes appropriate. As a result, the secondary hydroxyl group and the polar group represented by Q¹ to Q³ in the formulae (5-1) to (5-3) are less likely to be inhibited by other polar groups from bonding to the active point on the protective layer. In addition, the secondary hydroxyl group and the polar group represented by Q¹ to Q³ in the formulae (5-1) to (5-3) are less likely to aggregate.

Therefore, the secondary hydroxyl group and the polar group represented by Q¹ to Q³ in the formulae (5-1) to (5-3) can each be independently adsorbed to the active point on the protective layer. As a result, a lubricating layer containing the fluorine-containing ether compound having a terminal group in which Q¹ to Q³ in the formulae (5-1) to (5-3) are each a polar group has superior adhesion to the protective layer and excellent pickup resistance.

Particularly, Q¹ to Q³ in the formulae (5-1) to (5-3) are each preferably any of a hydroxyl group, a cyano group, and —CH═CH₂. This is because the fluorine-containing ether compound enables the formation of a lubricating layer having a higher coating rate, superior adhesion, and superior pickup resistance.

In the terminal group represented by the formula (5-1), a is 1 or 2, and b is an integer of 0 to 3. In a case where a is 1, Q¹ is a polar group, and the formula (5-1) has two polar groups. In this case, since the formula (5-1) has two polar groups, it is possible to form a lubricating layer having favorable adhesion to the protective layer. In a case where a is 2, Q¹ may be any of an aromatic hydrocarbon group, an unsaturated heterocyclic group, an alkenyl group, an alkynyl group, and a polar group. Even in a case where a is 2, and Q¹ is any of an aromatic hydrocarbon group, an unsaturated heterocyclic group, an alkenyl group, and an alkynyl group, the formula (5-1) has two polar groups. Therefore, it is possible to form a lubricating layer having favorable adhesion to the protective layer. Additionally, since Q¹ is an aromatic hydrocarbon group, an unsaturated heterocyclic group, an alkenyl group, or an alkynyl group, it is possible to form a lubricating layer having excellent adhesion and pickup resistance by a T-x interaction between the carbon-carbon unsaturated bond portion in Q¹ and the protective layer without impairing the adhesion of the two hydroxyl groups that are included in the formula (5-1) to the protective layer. In addition, in a case where a is 2 and Q¹ is a polar group, the formula (5-1) has three polar groups. Therefore, it is possible to form a lubricating layer exhibiting superior adhesion to the protective layer.

In the terminal group represented by the formula (5-1), b is an integer of 0 to 3. In the terminal group represented by the formula (5-1), even when Q¹ in the formula (5-1) is a polar group, the distance between Q¹ and the secondary hydroxyl group in the formula (5-1) does not become too close, and the polar groups in the formula (5-1) are thus less likely to aggregate. In a case where Q¹ in the formula (5-1) is a polar group, since the distance between Q¹ and the secondary hydroxyl group in the formula (5-1) becomes more appropriate, b is preferably 1 or more. In the terminal group represented by the formula (5-1), since b is 3 or less, the mobility of Q¹ in the formula (5-1) does not become too high, and each polar group that the terminal group has can be sufficiently adsorbed to the protective layer. b is more preferably 2 or less.

In the terminal group represented by the formula (5-2), c is an integer of 1 to 3. In a case where d is 0, Q² is a polar group. Since c is an integer of 1 or more, in a case where d is 0, the distance between Q² and the secondary hydroxyl group in the formula (5-2) becomes appropriate, and even Q² is a polar group, the polar groups in the formula (5-2) are less likely to aggregate. In addition, since c is an integer of 1 or more, in a case where d is 1, the distance between the secondary hydroxyl groups in the formula (5-2) does not become too close, and the secondary hydroxyl groups in the formula (5-2) are less likely to aggregate. In the terminal group represented by the formula (5-2), since c is 3 or less, the mobility of the terminal group represented by the formula (5-2) does not become too high, and each polar group that the terminal group has can be sufficiently adsorbed to the protective layer. c is preferably 2 or less.

In the terminal group represented by the formula (5-2), d is 0 or 1. In a case where d is 0, Q² is a polar group, and the formula (5-2) has two polar groups. In this case, since the formula (5-2) has two polar groups, it is possible to form a lubricating layer having favorable adhesion to the protective layer. In a case where d is 1, Q² may be any of an aromatic hydrocarbon group, an unsaturated heterocyclic group, an alkenyl group, an alkynyl group, and a polar group. Even in a case where d is 1, and Q² is any of an aromatic hydrocarbon group, an unsaturated heterocyclic group, an alkenyl group, and an alkynyl group, the formula (5-2) has two polar groups. Therefore, it is possible to form a lubricating layer having favorable adhesion to the protective layer. Additionally, since Q² is an aromatic hydrocarbon group, an unsaturated heterocyclic group, an alkenyl group, or an alkynyl group, it is possible to form a lubricating layer having excellent adhesion and pickup resistance by a x-x interaction between the carbon-carbon unsaturated bond portion in Q² and the protective layer without impairing the adhesion of the two hydroxyl groups that are included in the formula (5-2) to the protective layer. In addition, in a case where d is 1 and Q² is a polar group, the formula (5-2) has three polar groups. Therefore, it is possible to form a lubricating layer exhibiting excellent adhesion to the protective layer.

In the terminal group represented by the formula (5-2), e is an integer of 0 to 3. In the terminal group represented by the formula (5-2), even when Q² in the formula (5-2) is a polar group, the distance between Q² and the secondary hydroxyl group in the formula (5-2) does not become too close, and the polar groups in the formula (5-2) are thus less likely to aggregate. In a case where Q² in the formula (5-2) is a polar group, since the distance between Q² and the secondary hydroxyl group in the formula (5-2) becomes more appropriate, e is preferably 1 or more. In addition, in a case where d is 0, even when e is 0, the distance between Q², which is a polar group, and the secondary hydroxyl group in the formula (5-2) becomes more appropriate due to c methylene group. In a case where d is 0, when e is 1 or more, the distance between Q², which is a polar group, and the secondary hydroxyl group in the formula (5-2) becomes more appropriate due to c+e methylene groups, which is preferable. In the terminal group represented by the formula (5-2), since e is 3 or less, the mobility of Q² in the formula (5-2) does not become too high, and each polar group that the terminal group has can be sufficiently adsorbed to the protective layer. e is preferably 2 or less.

In the terminal group represented by the formula (5-3), g is 0 or 1. In a case where g is 0, Q³ is a polar group, and the formula (5-3) has two polar groups. In this case, since the formula (5-3) has two polar groups, it is possible to form a lubricating layer having favorable adhesion to the protective layer. In a case where g is 1, Q³ may be any of an aromatic hydrocarbon group, an unsaturated heterocyclic group, an alkenyl group, an alkynyl group, and a polar group. Even in a case where g is 1, and Q³ is any of an aromatic hydrocarbon group, an unsaturated heterocyclic group, an alkenyl group, and an alkynyl group, the formula (5-3) has two polar groups. Therefore, it is possible to form a lubricating layer having favorable adhesion to the protective layer. Additionally, since Q³ is an aromatic hydrocarbon group, an unsaturated heterocyclic group, an alkenyl group, or an alkynyl group, it is possible to form a lubricating layer having excellent adhesion and pickup resistance by a π-π interaction between the carbon-carbon unsaturated bond portion in Q³ and the protective layer without impairing the adhesion of the two hydroxyl groups that are included in the formula (5-3) to the protective layer. In addition, in a case where g is 1 and Q³ is a polar group, the formula (5-3) has three polar groups. Therefore, it is possible to form a lubricating layer exhibiting excellent adhesion to the protective layer.

In the terminal group represented by the formula (5-3), f is an integer of 1 to 3. Since f is 1 or more, in a case where g is 1, the distance between the secondary hydroxyl groups in the formula (5-3) does not become too close. Therefore, the secondary hydroxyl groups in the formula (5-3) are less likely to aggregate. In the terminal group represented by the formula (5-3), since f is 3 or less, the mobility of the terminal group represented by the formula (5-3) does not become too high, and each polar group that the terminal group has can be sufficiently adsorbed to the protective layer. f is preferably 2 or less.

In the terminal group represented by the formula (5-3), h is an integer of 0 to 3. Therefore, a carbon atom to which Q³ bonds and a carbon atom to which the secondary hydroxyl group adjacent to Q³ bonds directly bond to each other or bond to each other through a chain-like linking group of 1 to 3 atoms. Therefore, the distance between Q³ and the secondary hydroxyl group adjacent to Q³ becomes appropriate, Q³ and the secondary hydroxyl group are each likely to interact with the protective layer, and the adhesion to the protective layer becomes favorable. In the terminal group represented by the formula (5-3), since h is 3 or less, the mobility of Q³ in the formula (5-3) does not become too high, and each polar group that the terminal group has can be sufficiently adsorbed to the protective layer. h is preferably 0 to 2.

In the fluorine-containing ether compound represented by the formula (1), it is preferable that n is 0 and R¹ and R⁴ are identical to each other. This is because the fluorine-containing ether compound becomes easy to synthesize.

In the fluorine-containing ether compound represented by the formula (1), it is preferable that n is 1, R¹ and R⁴ are identical to each other, and two R²'s are identical to each other. This is because the fluorine-containing ether compound becomes easy to synthesize.

In the fluorine-containing ether compound represented by the formula (1), it is preferable that n is 2, R¹ and R⁴ are identical to each other, two R²'s are identical to each other, and three R²'s are identical to one another. This is because the fluorine-containing ether compound becomes easy to synthesize.

The fluorine-containing ether compound represented by the formula (1) is specifically preferably any of compounds represented by the following formulae (1A) to (1T) and (2A) to (2E). In a case where the compound represented by the formula (1) is any of the compounds represented by the following formulae (1A) to (1T) and (2A) to (2E), it becomes easy to obtain a raw material, and it is possible to form a lubricating layer having excellent adhesion, favorable chemical substance resistance and wear resistance, and superior pickup resistance even when the thickness is thin.

In all of the compounds represented by the following formulae (2A) to (2E), two perfluoropolyether chains represented by R² in the formula (1) are identical to each other. In the compounds represented by the following formulae (1A) to (1T) and (2A) to (2E), Rf₁ and Rf₂ representing the perfluoropolyether chain are the following structures, respectively. That is, in the compounds represented by the following formulae (1A) to (1D), (1K) to (1N), (2A), (2B), and (2D), Rf₁ is a perfluoropolyether chain represented by the following formula (6-1). In the compounds represented by the formulae (1E) to (1J), (10) to (1T), (2C), and (2E), Rf₂ is a perfluoropolyether chain represented by the following formula (6-2).

i and j in Rf₁ and k in Rf₂, which represent the perfluoropolyether chains in the formulae (1A) to (1T) and (2A) to (2E), are values indicating the average degrees of polymerization and thus do not necessarily become an integer.

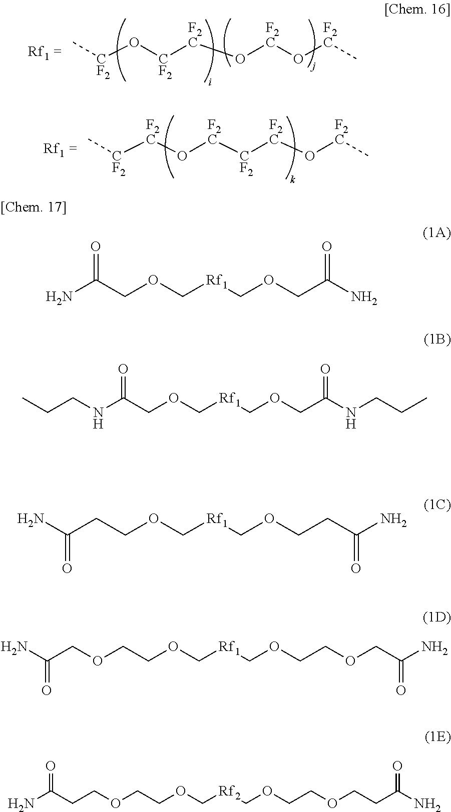

(in Rf₁ in the formula (1A), i and j indicate average degrees of polymerization, i represents 1 to 20, and j represents 0 to 20.)

(in Rf₁ in the formula (1B), i and j indicate average degrees of polymerization, i represents 1 to 20, and j represents 0 to 20.)

(in Rf₁ in the formula (1C), i and j indicate average degrees of polymerization, i represents 1 to 20, and j represents 0 to 20.)

(in Rf₁ in the formula (1D), i and j indicate average degrees of polymerization, i represents 1 to 20, and j represents 0 to 20.)

(in Rf₂ in the formula (1E), k indicates an average degree of polymerization and represents 1 to 15.)

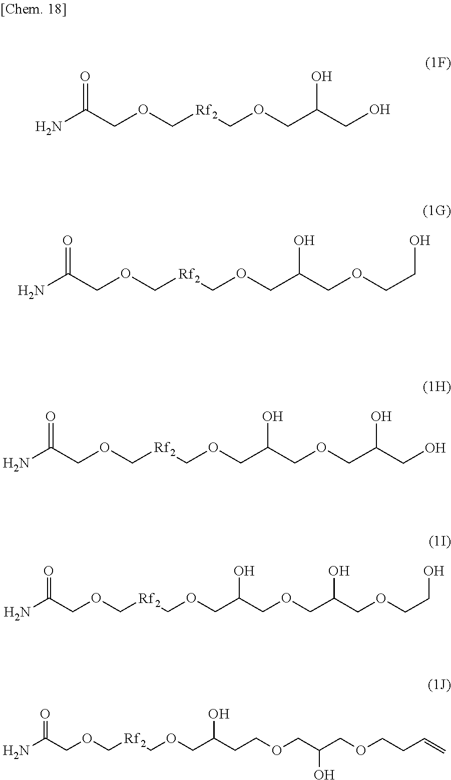

(in Rf₂ in the formula (1F), k indicates an average degree of polymerization and represents 1 to 15.)

(in Rf₂ in the formula (1G), k indicates an average degree of polymerization and represents 1 to 15.)

(in Rf₂ in the formula (1H), k indicates an average degree of polymerization and represents 1 to 15.)

(in Rf₂ in the formula (1I), k indicates an average degree of polymerization and represents 1 to 15.)

(in Rf₂ in the formula (1J), k indicates an average degree of polymerization and represents 1 to 15.)

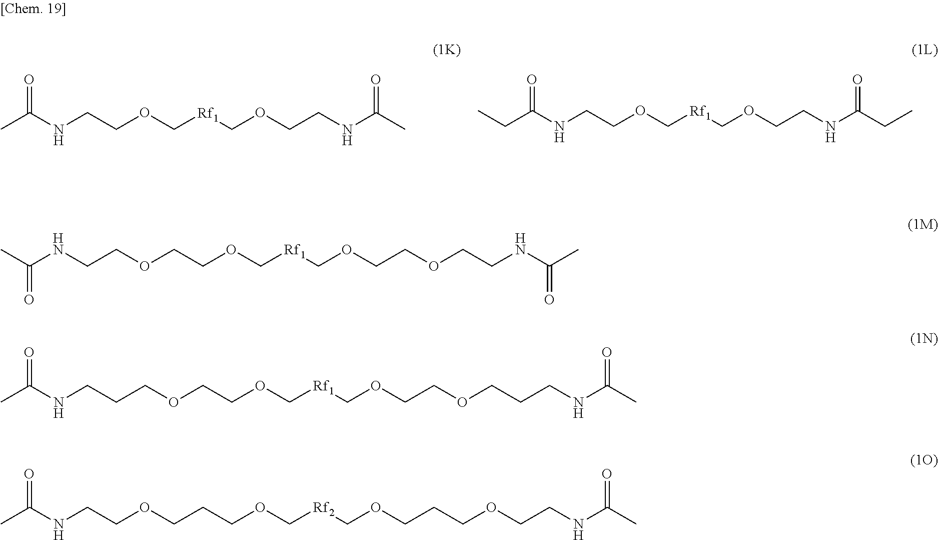

(in Rf₁ in the formula (1K), i and j indicate average degrees of polymerization, i represents 1 to 20, and j represents 0 to 20.)

(in Rf₁ in the formula (1L), i and j indicate average degrees of polymerization, i represents 1 to 20, and j represents 0 to 20.)

(in Rf₁ in the formula (1M), i and j indicate average degrees of polymerization, i represents 1 to 20, and j represents 0 to 20.)

(in Rf₁ in the formula (1N), i and j indicate average degrees of polymerization, i represents 1 to 20, and j represents 0 to 20.)

(in Rf₂ in the formula (1O), k indicates an average degree of polymerization and represents 1 to 15.)

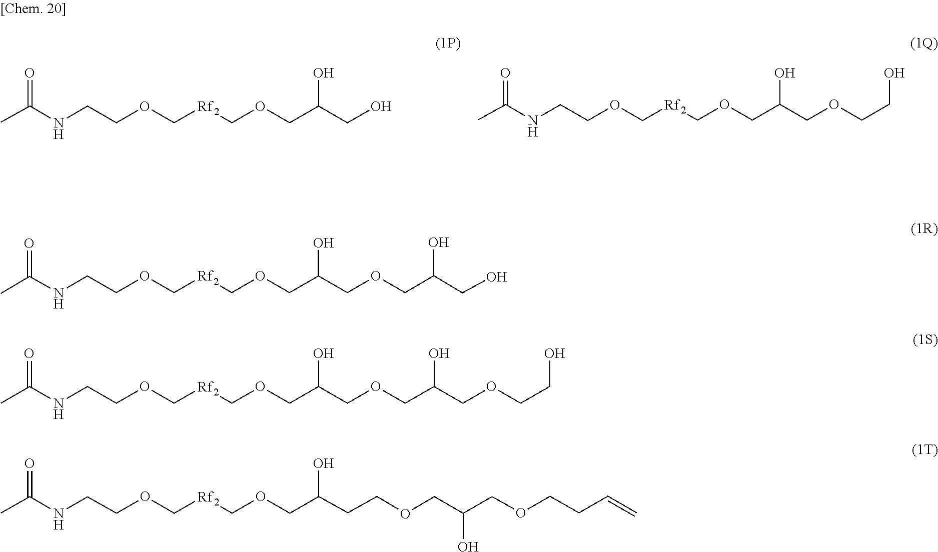

(in Rf₂ in the formula (1P), k indicates an average degree of polymerization and represents 1 to 15.)

(in Rf₂ in the formula (1Q), k indicates an average degree of polymerization and represents 1 to 15.)

(in Rf₂ in the formula (1R), k indicates an average degree of polymerization and represents 1 to 15.)

(in Rf₂ in the formula (1S), k indicates an average degree of polymerization and represents 1 to 15.)

(in Rf₂ in the formula (1T), k indicates an average degree of polymerization and represents 1 to 15.)

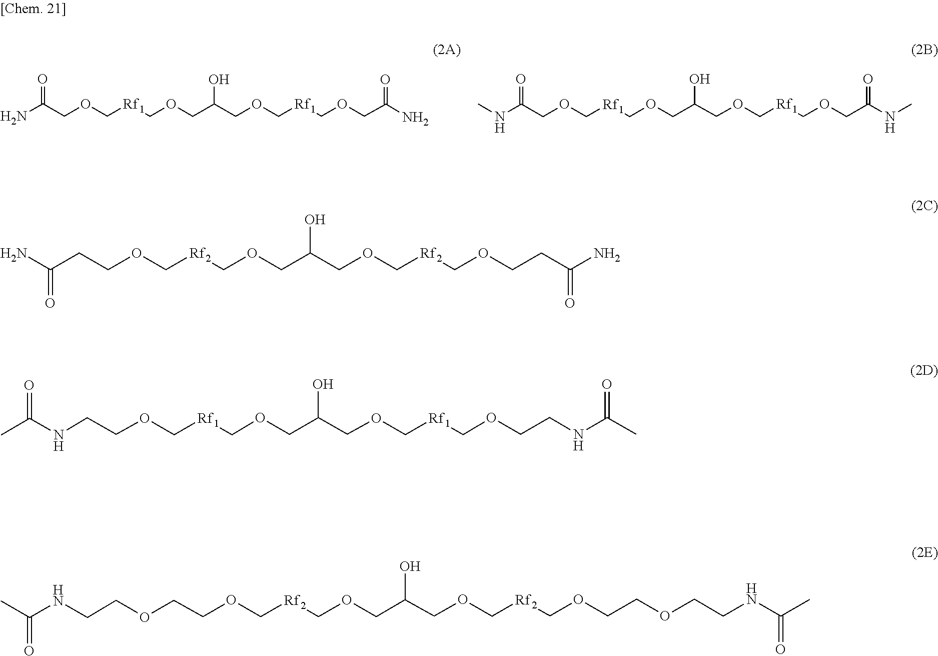

(in two Rf₁'s in the formula (2A), i and j indicate average degrees of polymerization, i represents 1 to 20, and j represents 0 to 20. In the two Rf₁'s, the average degrees of polymerization may be identical to each other or may be different from each other)

(in two Rf₁'s in the formula (2B), i and j indicate average degrees of polymerization, i represents 1 to 20, and j represents 0 to 20. In the two Rf₁'s, the average degrees of polymerization may be identical to each other or may be different from each other)

(in two Rf₂'s in the formula (2C), k indicates an average degree of polymerization and represents 1 to 15. In the two Rf₂'s, the average degrees of polymerization may be identical to each other or may be different from each other)

(in two Rf₁'s in the formula (2D), i and j indicate average degrees of polymerization, i represents 1 to 20, and j represents 0 to 20. In the two Rf₁'s, the average degrees of polymerization may be identical to each other or may be different from each other)

(in two Rf₂'s in the formula (2E), k indicates an average degree of polymerization and represents 1 to 15. In the two Rf₂'s, the average degrees of polymerization may be identical to each other or may be different from each other)

The number-average molecular weight (Mn) of the fluorine-containing ether compound of the present embodiment is preferably within a range of 500 to 10000, more preferably within a range of 500 to 5000, and particularly preferably within a range of 1000 to 3000. When the number-average molecular weight is 500 or more, a lubricant containing the fluorine-containing ether compound of the present embodiment is less likely to transpire, and it is possible to prevent the lubricant from transpiring and transferring to a magnetic head. In addition, when the number-average molecular weight is 10000 or less, the viscosity of the fluorine-containing ether compound becomes appropriate, and it is possible to easily form a lubricating layer of thin thickness by applying a lubricant containing this fluorine-containing ether compound. When the number-average molecular weight is 5000 or less, the viscosity becomes easy to handle in the case of applying the fluorine-containing ether compound to lubricants, which is more preferable.

The number-average molecular weight (Mn) of the fluorine-containing ether compound is a value measured by ¹H-NMR and ¹⁹F-NMR with AVANCE III 400 manufactured by Bruker Biospin Group. Specifically, the number of repeating units of the PFPE chain is calculated from an integral value measured by ¹⁹F-NMR, and the number-average molecular weight is obtained. In the NMR (nuclear magnetic resonance) measurement, a sample was diluted into a single solvent or solvent mixture of hexafluorobenzene, d-acetone, d-tetrahydrofuran, and the like and used in the measurement. As ¹⁹F-NMR chemical shift standards, the peak of hexafluorobenzene was set to −164.7 ppm. As ¹H-NMR chemical shift standards, the peak of acetone was set to 2.2 ppm.

For the fluorine-containing ether compound of the present embodiment, the molecular weight dispersity (weight-average molecular weight (Mw)/number-average molecular weight (Mn) ratio) is preferably made to be 1.3 or less by molecular weight fractionation by an appropriate method.

In the present embodiment, a method for the molecular weight fractionation is not particularly limited, and it is possible to use, for example, molecular weight fractionation by silica gel column chromatography, gel permeation chromatography (GPC), or the like, molecular weight fractionation by a supercritical extraction method, and the like.

### Production Method

A method for producing the fluorine-containing ether compound of the present embodiment is not particularly limited, and the fluorine-containing ether compound can be produced by a known conventional method. The fluorine-containing ether compound of the present embodiment can be produced using, for example, a production method to be described below.

### First Production Method

In the case of producing a compound in which n in the formula (1) is 0 and R¹ and R⁴ are identical to each other, it is possible to use a production method indicated by the following formula (7).

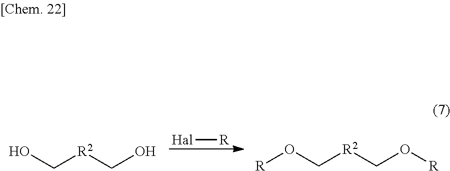

(in the formula (7), R² is identical to R² in the formula (1). Hal represents a (pseudo)halogen group. R is a group corresponding to the formula (2-1) or the formula (2-2).)

A fluorine-based compound having hydroxymethyl groups (—CH₂OH) at both terminals of a perfluoropolyether chain corresponding to R² in the formula (1) is prepared. An alkyl (pseudo)halide compound having an amide bond portion and including a group corresponding to the formula (2-1) or the formula (2-2) is reacted with the hydroxymethyl groups at both terminals of the fluorine-based compound. The reaction ratio between the fluorine-based compound having hydroxymethyl groups at both terminals and the alkyl (pseudo)halide compound is preferably approximately 1:2 (mole ratio). This makes it possible to produce a fluorine-containing ether compound in which n in the formula (1) is 0 and R¹ and R⁴ are identical to each other.

The alkyl (pseudo)halide compound having an amide bond portion that is used in the first production method is a compound including a group corresponding to the formula (2-1) or the formula (2-2), and examples thereof include compounds indicated by the following formulae (2-1-a) to (2-1-i) and (2-2-a) to (2-2-i). The compounds indicated by the formulae (2-1-a) to (2-1-i) and (2-2-a) to (2-2-i) can be produced by known methods, and commercially available products may also be used. —NH—, —NH₂—, and the —OH group that form the amide bond, which are included in the compounds indicated by the formulae (2-1-a) to (2-1-i) and (2-2-a) to (2-2-i), may be used for a reaction after being protected with an appropriate protective group.

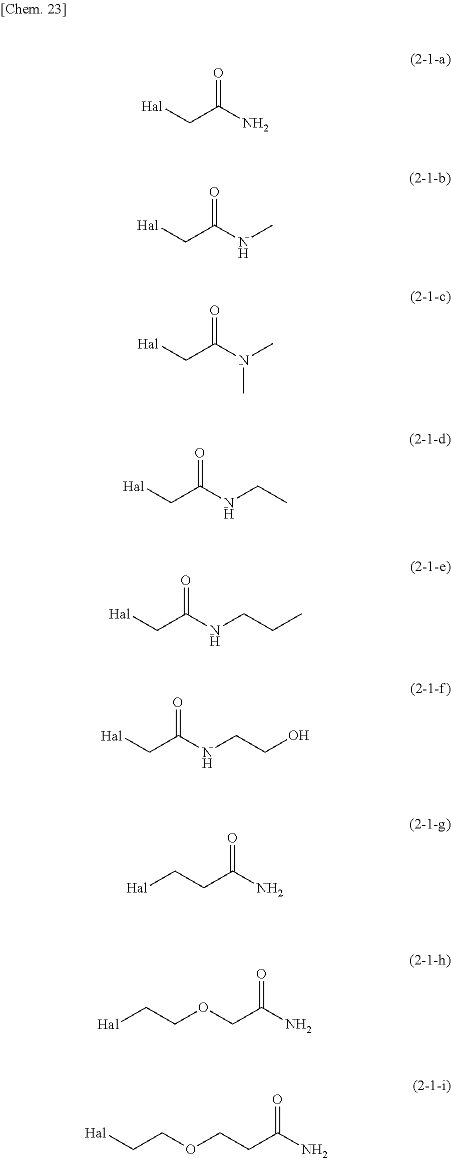

(in the formula (2-1-a) to (2-1-i), Hal represents a (pseudo)halogen group.)

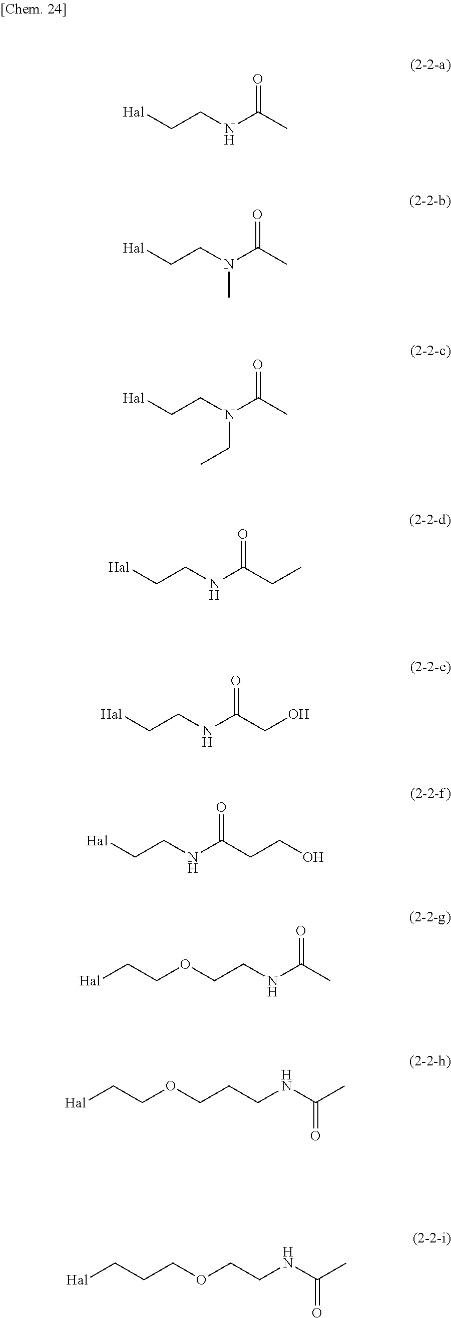

(in the formula (2-2-a) to (2-2-i), Hal represents a (pseudo)halogen group.)

### Second Production Method

In the case of producing a compound in which n in the formula (1) is 0 and R¹ and R⁴ are different from each other, it is possible to use a production method indicated by the following formula (8).

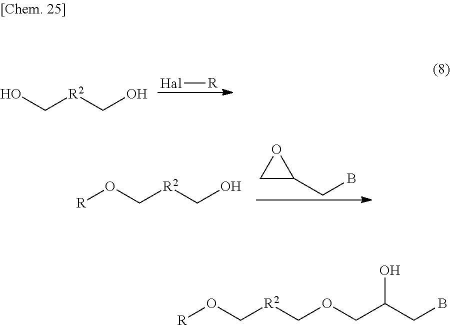

(in the formula (8), R² is identical to R² in the formula (1). Hal represents a (pseudo)halogen group. R is a group corresponding to the formula (2-1) or the formula (2-2). B represents a partial structure of the terminal group represented by R⁴.)

### First Reaction

A fluorine-based compound having hydroxymethyl groups (—CH₂OH) at both terminals of a perfluoropolyether chain corresponding to R² in the formula (1) is prepared. An alkyl (pseudo)halide compound having an amide bond portion is reacted with the hydroxymethyl group at one terminal of the fluorine-based compound. The reaction ratio between the fluorine-based compound having a hydroxymethyl group at one terminal and the alkyl (pseudo)halide compound is preferably approximately 1:1 (mole ratio). This produces a first intermediate compound in which an organic group having an amide bond portion corresponding to R¹ has bonded to one terminal of the PFPE chain corresponding to R².

As the alkyl (pseudo)halide compound having an amide bond portion that is used in the second production method, the same compound as the alkyl (pseudo)halide compound that is used in the first production method can be used.

### Second Reaction

Next, an epoxy compound having the partial structure of R⁴ in the formula (1) is reacted with the hydroxymethyl groups (—CH₂OH) at the other terminal in the first intermediate compound obtained by the first reaction. The reaction ratio between the first intermediate compound and the epoxy compound having the partial structure of R⁴ is preferably approximately 1:1 (mole ratio). This makes it possible to produce a fluorine-containing ether compound in which n in the formula (1) is 0 and R¹ and R⁴ are different from each other. The epoxy compound having the partial structure of R⁴ can be produced by a method to be described below.

In the present embodiment, one terminal of the perfluoropolyether chain and the alkyl (pseudo)halide compound having an amide bond portion are reacted with each other in the first reaction, and the other terminal of the perfluoropolyether chain and the epoxy compound having the partial structure of R⁴ are reacted with each other in the second reaction, but the perfluoropolyether chain may be reacted with the epoxy compound having the partial structure of R⁴ in the first reaction and reacted with the alkyl (pseudo)halide compound having an amide bond portion in the second reaction.

In the case of a group in which R¹ and R⁴ are different from each other and R⁴ is a group represented by the formula (2-1) or the formula (2-2), an alkyl (pseudo)halide compound having an amide bond portion corresponding to R⁴ may be used instead of the epoxy compound having the partial structure of R⁴ in the second reaction.

### Third Production Method

In the case of producing a compound in which n in the formula (1) is 1, and R¹ and R⁴ are identical to each other, and two R²′ are identical to each other, it is possible to use a production method indicated by the following formula (9).

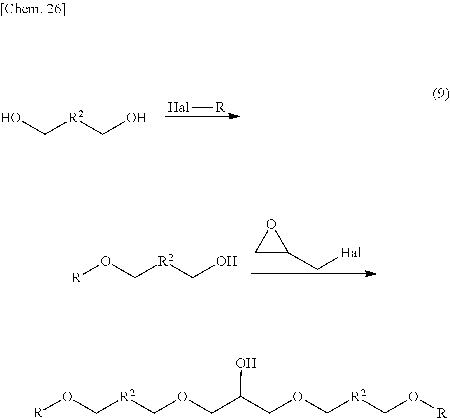

(in the formula (9), R² is identical to R² in the formula (1). Hal represents a (pseudo)halogen group. R is a group corresponding to the formula (2-1) or the formula (2-2).)

### First Reaction

In a first reaction of the third production method, a first intermediate compound in which organic groups having an amide bond portion corresponding to R¹ has bonded to one terminal of the PFPE chain corresponding to R² is produced in the same manner as in the first reaction of the second production method.

### Second Reaction

Next, an epoxy compound having a group with the partial structure of the linking group indicated by R³ in the formula (1) and a (pseudo)halogen group is reacted with the hydroxymethyl groups (—CH₂OH) at the other terminal in the first intermediate compound obtained by the first reaction. The reaction ratio between the first intermediate compound and this epoxy compound is preferably approximately 2:1 (mole ratio). This makes it possible to produce a fluorine-containing ether compound in which n in the formula (1) is 1, and R¹ and R⁴ are identical to each other, and two R²′ are identical to each other.

Examples of the epoxy compound having a group with the partial structure of the linking group indicated by R³ in the formula (1) and a (pseudo)halogen group (hereinafter referred to as the (pseudo)halogenated epoxy compound in some cases) include compounds represented by the following formulae (4-A) to (4-C).

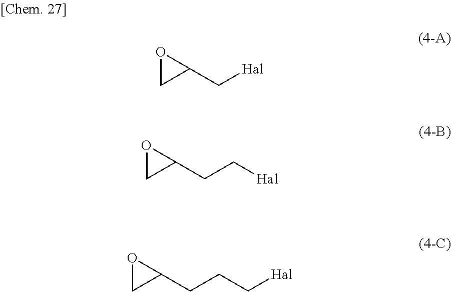

(in the formulae (4-A) to (4-C), Hal represents a (pseudo)halogen group.)

### Fourth Production Method

In the case of producing a compound in which n in the formula (1) is 1 and R¹ and R⁴ are different from each other or two R²'s are different from each other, it is possible to use a production method indicated by the following formula (1O).

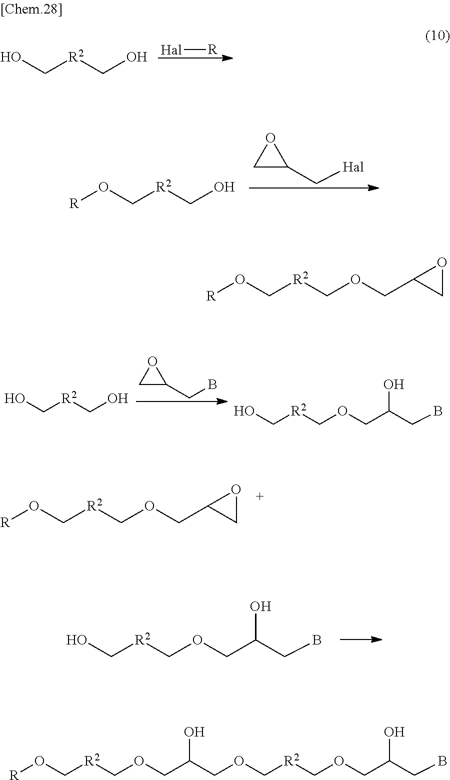

(in the formula (1O), R² is identical to R² in the formula (1), and two R²'s may be identical to or different from each other. Hal represents a (pseudo)halogen group. R is a group corresponding to the formula (2-1) or the formula (2-2). B represents a partial structure of the terminal group represented by R⁴.)

### First Reaction

In a first reaction of the fourth production method, a fluorine-based compound having hydroxymethyl groups (—CH₂OH) at both terminals of a perfluoropolyether chain corresponding to R² on the R¹ side in the formula (1) is prepared, and a first intermediate compound in which an organic group having an amide bond portion corresponding to R¹ has bonded to one terminal of the PFPE chain corresponding to R² on the R¹ side is produced in the same manner as in the first reaction of the second production method.

### Second Reaction

Next, a (pseudo)halogenated epoxy compound is reacted with the hydroxymethyl group (—CH₂OH) at the other terminal in the first intermediate compound obtained by the first reaction. The reaction ratio between the first intermediate compound and the (pseudo)halogenated epoxy compound is preferably approximately 1:1 (mole ratio). This produces a second intermediate compound in which an organic group having an amide bond portion corresponding to R¹ bonds to one terminal of the PFPE chain corresponding to R² on the R¹ side and an epoxy group having a partial structure of the linking group represented by R³ has bonded to the other terminal.

As the (pseudo)halogenated epoxy compound that is used in the fourth production method, the same compound as the (pseudo)halogenated epoxy compound that is used in the third production method can be used.

### Third Reaction

A fluorine-based compound having hydroxymethyl groups (—CH₂OH) at both terminals of a perfluoropolyether chain corresponding to R² on the R⁴ side in the formula (1) is prepared. An epoxy compound having the partial structure of R⁴ in the formula (1) is reacted with a hydroxymethyl group at one terminal of the fluorine-based compound. The reaction ratio between the fluorine-based compound having hydroxymethyl groups at both terminals and the epoxy compound having the partial structure of R⁴ is preferably approximately 1:1 (mole ratio). This produces a third intermediate compound in which an organic group corresponding to R⁴ has bonded to one terminal of the PFPE chain corresponding to R² on the R⁴ side.

As the epoxy compound having the partial structure of R⁴ that is used in the fourth production method, the same compound as the epoxy compound that is used in the second production method can be used.

In the case of a group in which R¹ and R⁴ are different from each other and R⁴ is a group represented by the formula (2-1) or the formula (2-2), an alkyl (pseudo)halide compound having an amide bond portion corresponding to R⁴ may be used instead of the epoxy compound having the partial structure of R⁴ in the third reaction.

### Fourth Reaction

Finally, the second intermediate compound obtained by the second reaction and the third intermediate compound obtained by the third reaction are reacted with each other. The reaction ratio between the second intermediate compound and the third intermediate compound is preferably approximately 1:1 (mole ratio). This makes it possible to produce a fluorine-containing ether compound in which n in the formula (1) is 1 and R¹ and R⁴ are different from each other or two R²'s are different from each other.

In the fourth production method, the third reaction is performed after the second reaction, but the first and second reactions may be performed after the third reaction. In addition, in the fourth production method, the second intermediate compound is obtained by reacting the (pseudo)halogenated epoxy compound with the first intermediate compound, but a compound obtained by reacting the (pseudo)halogenated epoxy compound with the third intermediate compound may be reacted with the first intermediate compound.

### Fifth Production Method

In the case of producing a compound in which n in the formula (1) is 2, three perfluoropolyether chains represented by R² are identical to one another, two linking groups R³ are identical to each other, and the terminal groups R¹ and R⁴ are identical to each other or in the case of producing a compound in which n in the formula (1) is 2, and only the central R² in the three perfluoropolyether chains represented by R² is different, two linking groups R³ are identical to each other, and the terminal groups R¹ and R⁴ are identical to each other, it is possible to use a production method to be described below.

### First Reaction

A fluorine-based compound having hydroxymethyl groups (—CH₂OH) disposed at both terminals of a perfluoropolyether chain corresponding to R² at the molecular center in the formula (1), respectively, is prepared. Next, the hydroxyl groups of the hydroxymethyl groups disposed at both terminals of the fluorine-based compound are reacted with a (pseudo)halogenated epoxy compound. The reaction ratio between the fluorine-based compound and the (pseudo)halogenated epoxy compound is preferably approximately 1:2 (mole ratio). This produces a first intermediate compound having epoxy groups having a partial structure of the linking group represented by R³ at both terminals of the perfluoropolyether chain corresponding to R² at the molecular center in the formula (1).

As the (pseudo)halogenated epoxy compound that is used in the fifth production method, the same compound as the (pseudo)halogenated epoxy compound that is used in the third production method can be used.

### Second Reaction

In a second reaction of the fifth production method, a fluorine-based compound having hydroxymethyl groups (—CH₂OH) at both terminals of a perfluoropolyether chain corresponding to R² on the R¹ side (=R⁴ side) in the formula (1) is prepared. A second intermediate compound in which an organic group having an amide bond portion corresponding to R¹ (=R⁴) is bonded to one terminal of the PFPE chain corresponding to R² on the R¹ side (=R⁴ side) is produced in the same manner as in the first reaction of the second production method.

### Third Reaction

Finally, the first intermediate compound obtained by the first reaction and the second intermediate compound obtained by the second reaction are reacted with each other. The reaction ratio between the first intermediate compound and the second intermediate compound is preferably approximately 1:2 (mole ratio). This makes it possible to produce a compound in which n in the formula (1) is 2, three perfluoropolyether chains represented by R² are identical to one another, two linking groups R³ are identical to each other, and the terminal groups R¹ and R⁴ are identical to each other or a fluorine-containing ether compound in which n in the formula (1) is 2, and only the central R² in the three perfluoropolyether chains indicated by R² is different, two linking groups R³ are identical to each other, and the terminal groups R¹ and R⁴ are identical to each other.

In the fifth production method, the second reaction is performed after the first reaction, but the first reaction may be performed after the second reaction.

### Sixth Production Method

In the case of producing a compound in which n in the formula (1) is 2, two linking groups R³ are identical to each other, and any one or more of the terminal groups R¹ and R⁴, and the R² on the R¹ side and the R² on the R⁴ side (R² that is not positioned at the molecular center) in three perfluoropolyether chains are different, it is possible to use a production method to be described below.

### First Reaction

In a first reaction of the sixth production method, a first intermediate compound having epoxy groups with a partial structure of the linking group represented by R³ at both terminals of a perfluoropolyether chain corresponding to R² at the molecular center in the formula (1) is produced in the same manner as in the first reaction of the fifth production method.

### Second Reaction

A fluorine-based compound having hydroxymethyl groups (—CH₂OH) at both terminals of a perfluoropolyether chain corresponding to R² on the R¹ side in the formula (1) is prepared. An alkyl (pseudo)halide compound having an amide bond portion is reacted with a hydroxymethyl group at one terminal of the fluorine-based compound.

The reaction ratio between the fluorine-based compound having hydroxymethyl groups at both terminals and the alkyl (pseudo)halide compound is preferably approximately 1:1 (mole ratio). This produces a second intermediate compound in which an organic group having an amide bond portion corresponding to R¹ has bonded to one terminal of the PFPE chain corresponding to R² on the R¹ side.

As the alkyl (pseudo)halide compound having an amide bond portion that is used in the sixth production method, the same compound as the alkyl (pseudo)halide compound that is used in the first production method can be used.

### Third Reaction

In a third reaction of the sixth production method, a third intermediate compound in which an organic group corresponding to R⁴ has bonded to one terminal of the PFPE chain corresponding to R² on the R⁴ side is produced in the same manner as in the third reaction of the fourth production method.

### Fourth Reaction

The first intermediate compound obtained by the first reaction and the second intermediate compound obtained by the second reaction are reacted with each other. The reaction ratio between the first intermediate compound and the second intermediate compound is preferably approximately 1:1 (mole ratio). This produces a fourth intermediate compound having a skeleton in which R² on the R¹ side and R² at the molecular center in the formula (1) have bonded to each other through the linking group R³ and having an epoxy group with an organic group having an amide bond portion corresponding to R¹ bonding to one terminal of R² on the R¹ side and a partial structure of the linking group represented by R³ at the other terminal.

### Fifth Reaction

The fourth intermediate compound obtained by the fourth reaction and the third intermediate compound obtained by the third reaction are reacted with each other. The reaction ratio between the fourth intermediate compound and the third intermediate compound is preferably approximately 1:1 (mole ratio). This makes it possible to produce a fluorine-containing ether compound in which in which n in the formula (1) is 2, two linking groups R³ are identical to each other, and any one or more of the terminal groups R¹ and R⁴, and the R² on the R¹ side and the R² on the R⁴ side (R² that is not positioned at the molecular center) in three perfluoropolyether chains are different.

In the sixth production method, the first reaction to the third reaction are performed in this order, but the order may be changed. In addition, the fourth intermediate compound is produced by reacting the first intermediate compound and the second intermediate compound, but a fluorine-containing ether compound may be produced by reacting the second intermediate compound with an intermediate compound obtained by reacting the first intermediate compound and the third intermediate compound.

As the (pseudo)halogen group (X) that is included in the compounds used in the first production method to the sixth production method, for example, a halogeno group, an arylsulfonyloxy group, or an alkylsulfonyloxy group optionally having a fluorine atom as a substituent, and the like can be used. More specifically, as the (pseudo)halogen group (X), it is possible to use at least one selected from a chloro group, a bromo group, an iodo group, a p-toluenesulfonyloxy group, a methanesulfonyloxy group, a trifluoromethanesulfonyloxy group, a perfluoroalkylsulfonyloxy group, and a nitrobenzenesulfonyloxy group or the like.

The amide bond portions and/or the polar groups serving as the partial structures of R¹ and/or R⁴ that are included in the compounds and the intermediate compounds that are used in the first production method to the sixth production methods described above may be protected by appropriate protective groups. In addition, the protective groups that are included in the above-described compounds and intermediate compounds can be deprotected at appropriate stages.

### Method for Producing Epoxy Compound Having Partial Structure of R⁴

The epoxy compound having the partial structure of R⁴ in the formula (1) can be produced using, for example, any of (Method 1) to (Method 3) to be described below.

### Method 1

As shown in the following formula (1I), the epoxy compound can be produced using a method in which an alcohol (B′—OH) having the partial structure of the terminal group represented by R⁴ in the formula (1) and a halogen compound having an epoxy group are reacted with each other.

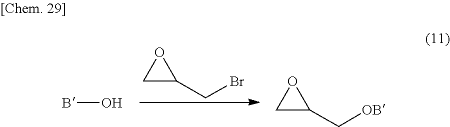

(in the formula (1I), B′ represents the partial structure of the terminal group represented by R⁴ in the formula (1).)

### Method 2

As shown in the following formula (12), the epoxy compound can be produced using a method in which an alcohol (B′—OH) having the partial structure of the terminal group represented by R⁴ in the formula (1) and allyl glycidyl ether are addition-reacted with each other, and an unsaturated bond portion that is included in a compound obtained by the addition reaction is then oxidized by making m-chloroperbenzoic acid (mCPBA) act thereon.

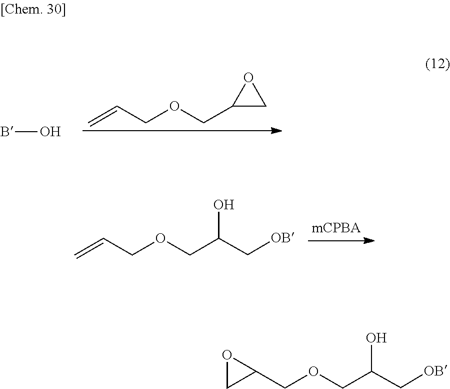

(in the formula (12), B′ represents the partial structure of the terminal group represented by R⁴ in the formula (1). mCPBA represents m-chloroperbenzoic acid.)

### Method 3

As shown in the following formula (13), the epoxy compound can be produced using a method in which an alcohol (B′—OH) having the partial structure of the terminal group represented by R⁴ in the formula (1) and a halogen compound having an alkenyl group are reacted with each other, and an unsaturated bond portion that is included in the obtained compound is oxidized by making m-chloroperbenzoic acid (mCPBA) act thereon.

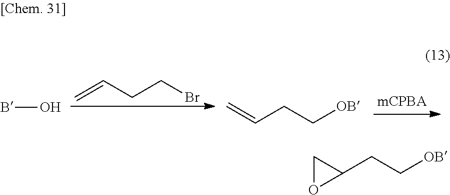

(in the formula (13), B′ represents the partial structure of the terminal group represented by R⁴ in the formula (1). mCPBA represents m-chloroperbenzoic acid.)

As the epoxy compound having the partial structure of R⁴, a commercially available product may also be purchased and used.

### Lubricant for Magnetic Recording Medium

A lubricant for a magnetic recording medium of the present embodiment contains the fluorine-containing ether compound represented by the formula (1).

The lubricant of the present embodiment can be used after being mixed with a known material that is used as a material for lubricants as necessary as long as characteristics attributed to the fluorine-containing ether compound represented by the formula (1) are not impaired.

Specific examples of the known material include FOMBLIN (registered trademark) ZDIAC, FOMBLIN ZDEAL, and FOMBLIN AM-2001 (all manufactured by Solvay Solexis, Inc.), Moresco A20H (manufactured by Moresco Corporation), and the like. For the known material that is used after being mixed with the lubricant of the present embodiment, the number-average molecular weight is preferably 500 to 10000.

In a case where the lubricant of the present embodiment contains a material other than the fluorine-containing ether compound represented by the formula (1), the content of the fluorine-containing ether compound represented by the formula (1) in the lubricant of the present embodiment is preferably 50 mass % or more and more preferably 70 mass % or more. The content of the fluorine-containing ether compound represented by the formula (1) may be 80 mass % or more or 90 mass % or more.

The lubricant of the present embodiment contains the fluorine-containing ether compound represented by the formula (1) and is thus capable of forming a lubricating layer having excellent adhesion to the protective layer, being capable of coating the surface of the protective layer with a high coating rate, and having favorable coatability even when the thickness is made to be thin. Therefore, the lubricant of the present embodiment makes it possible to form a lubricating layer that provides favorable chemical substance resistance and wear resistance to the magnetic recording medium and has excellent pickup-curbing effect even when the thickness is made to be thin.

### Magnetic Recording Medium

A magnetic recording medium of the present embodiment has at least a magnetic layer, a protective layer, and a lubricating layer provided in order on a substrate.

The magnetic recording medium of the present embodiment can be provided with one or more base layers between the substrate and the magnetic layer as necessary. In addition, an adhesive layer and/or a soft magnetic layer can also be provided between the base layer and the substrate.

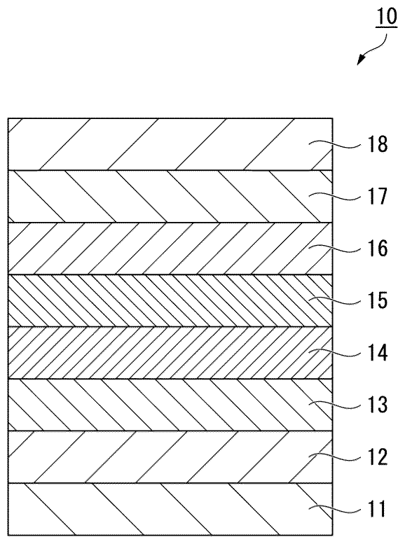

FIG. 1 is a schematic cross-sectional view showing one embodiment of a magnetic recording medium of the present invention.

A magnetic recording medium 10 of the present embodiment forms a structure in which an adhesive layer 12, a soft magnetic layer 13, a first base layer 14, a second base layer 15, a magnetic layer 16, a protective layer 17, and a lubricating layer 18 are provided in order on a substrate 11.

### Substrate

As the substrate 11, it is possible to use, for example, a non-magnetic substrate having a film made of NiP or a NiP alloy formed on a base body made of a metal or alloy material, such as Al or an Al alloy or the like.

In addition, as the substrate 11, a non-magnetic substrate made of a non-metal material, such as glass, ceramic, silicon, silicon carbide, carbon, or a resin, may be used or a non-magnetic substrate having a NiP or NiP alloy film formed on a base body made of the above-described non-metal material may be used.

### Adhesive Layer

The adhesive layer 12 prevents the progress of corrosion of the substrate 11, which is caused in a case where the substrate 11 and the soft magnetic layer 13 provided on the adhesive layer 12 are disposed in contact with each other.

The material of the adhesive layer 12 can be appropriately selected from, for example, Cr, a Cr alloy, Ti, a Ti alloy, CrTi, NiAl, an AlRu alloy, and the like. The adhesive layer 12 can be formed by, for example, a sputtering method.

### Soft Magnetic Layer

The soft magnetic layer 13 preferably has a structure in which a first soft magnetic film, an intermediate layer made of a Ru film, and a second soft magnetic film are laminated in order. That is, the soft magnetic layer 13 preferably has a structure in which an intermediate layer made of a Ru film is inserted between two layers of soft magnetic films, whereby the soft magnetic films on and under the intermediate layer are antiferromagnetically coupled (AFC).

Examples of the material of the first soft magnetic film and the second soft magnetic film include a CoZrTa alloy, a CoFe alloy, and the like.

To the CoFe alloy that is used for the first soft magnetic film and the second soft magnetic film, any of Zr, Ta, and Nb is preferably added. This accelerates the amorphization of the first soft magnetic film and the second soft magnetic film. As a result, it becomes possible to improve the orientation of the first base layer (seed layer), and it becomes possible to reduce the flying height of a magnetic head.

The soft magnetic layer 13 can be formed by, for example, a sputtering method.

### First Base Layer

The first base layer 14 is a layer for controlling the orientation and crystal sizes of the second base layer 15 and the magnetic layer 16 provided thereon.

Examples of the first base layer 14 include layers made of a Cr layer, a Ta layer, a Ru layer, a CrMo alloy layer, a CoW alloy layer, a CrW alloy layer, a CrV alloy layer, a CrTi alloy layer, or the like.

The first base layer 14 can be formed by, for example, a sputtering method.

### Second Base Layer

The second base layer 15 is a layer for controlling the orientation of the magnetic layer 16 to be favorable. The second base layer 15 is preferably a layer made of Ru or a Ru alloy.

The second base layer 15 may be a single layer or may be composed of a plurality of layers. In a case where the second base layer 15 is composed of a plurality of layers, all of the layers may be made of the same material, or at least one layer may be made of a different material.

The second base layer 15 can be formed by, for example, a sputtering method.

### Magnetic Layer

The magnetic layer 16 is made of a magnetic film having an axis of easy magnetization vertically or horizontally oriented with respect to the substrate surface. The magnetic layer 16 is a layer containing Co and Pt. The magnetic layer 16 may be a layer containing an oxide, Cr, B, Cu, Ta, Zr, or the like to improve SNR characteristics.

Examples of the oxide that is contained in the magnetic layer 16 include SiO₂, SiO, Cr₂O₃, CoO, Ta₂O₃, TiO₂, and the like.

The magnetic layer 16 may be composed of a single layer or may be composed of a plurality of magnetic layers made of materials having different compositions.

For example, in a case where the magnetic layer 16 is composed of three layers of a first magnetic layer, a second magnetic layer, and a third magnetic layer in order from the bottom, the first magnetic layer is preferably a granular structure made of a material containing Co, Cr, and Pt and further containing an oxide. As the oxide that is contained in the first magnetic layer, for example, oxides of Cr, Si, Ta, Al, Ti, Mg, Co, and the like are preferably used. Among them, in particular, TiO₂, Cr₂O₃, SiO₂, and the like can be suitably used. In addition, the first magnetic layer is preferably made of a composite oxide to which two or more kinds of oxides have been added. Among them, in particular, Cr₂O₃—SiO₂, Cr₂O₃—TiO₂, SiO₂—TiO₂, and the like can be suitably used.

The first magnetic layer may contain, aside from Co, Cr, Pt, and the oxide, one or more elements selected from B, Ta, Mo, Cu, Nd, W, Nb, Sm, Tb, Ru, and Re.

For the second magnetic layer, the same material as for the first magnetic layer can be used. The second magnetic layer is preferably a granular structure.

The third magnetic layer is preferably a non-granular structure made of a material containing Co, Cr, and Pt but not containing any oxides. The third magnetic layer may contain, aside from Co, Cr, and Pt, one or more elements selected from B, Ta, Mo, Cu, Nd, W, Nb, Sm, Tb, Ru, Re, and Mn.

In a case where the magnetic layer 16 is formed of a plurality of magnetic layers, it is preferable to provide a non-magnetic layer between the magnetic layers adjacent to each other. In a case where the magnetic layer 16 is composed of three layers of a first magnetic layer, a second magnetic layer, and a third magnetic layer, it is preferable to provide a non-magnetic layer between the first magnetic layer and the second magnetic layer and a non-magnetic layer between the second magnetic layer and the third magnetic layer.

For the non-magnetic layer that is provided between the magnetic layers adjacent to each other in the magnetic layer 16, it is possible to suitably use, for example, Ru, a Ru alloy, a CoCr alloy, a CoCrX1 alloy (X1 represents one or more elements selected from Pt, Ta, Zr, Re, Ru, Cu, Nb, Ni, Mn, Ge, Si, O, N, W, Mo, Ti, V, and B).

For the non-magnetic layer that is provided between the magnetic layers adjacent to each other in the magnetic layer 16, it is preferable to use an alloy material containing an oxide, a metal nitride, or a metal carbide. Specifically, as the oxide, it is possible to use, for example, SiO₂, Al₂O₃, Ta₂O₅, Cr₂O₃, MgO, Y₂O₃, TiO₂, and the like. As the metal nitride, it is possible to use, for example, AlN, Si₃N₄, TaN, CrN, and the like. As the metal carbide, it is possible to use, for example, TaC, BC, SiC, and the like.

The non-magnetic layer can be formed by, for example, a sputtering method.

In order to realize a higher recording density, the magnetic layer 16 is preferably a magnetic layer for perpendicular magnetic recording in which the axis of easy magnetization is vertically oriented with respect to the substrate surface. The magnetic layer 16 may be a magnetic layer for longitudinal magnetic recording.

The magnetic layer 16 may be formed by any known conventional method, such as a vapor deposition method, an ion beam sputtering method, or a magnetron sputtering method. The magnetic layer 16 is normally formed by a sputtering method.

### Protective Layer

The protective layer 17 protects the magnetic layer 16. The protective layer 17 may be a single layer or may be composed of a plurality of layers. Examples of the material of the protective layer 17 include carbon, nitrogen-containing carbon, silicon carbide, and the like. As the protective layer 17, a carbon-based protective layer can be preferably used, and an amorphous carbon protective layer is particularly preferable. When the protective layer 17 is a carbon-based protective layer, the interaction with the polar group that is contained in the fluorine-containing ether compound in the lubricating layer 18 is further enhanced, which is preferable.

The attachment force between the carbon-based protective layer and the lubricating layer 18 can be controlled by making the carbon-based protective layer with hydrogenated carbon and/or nitrided carbon and saving the hydrogen content and/or the nitrogen content in the carbon-based protective layer. The hydrogen content in the carbon-based protective layer is preferably 3 atomic % to 20 atomic % when measured by hydrogen forward scattering (HFS). In addition, the nitrogen content in the carbon-based protective layer is preferably 4 atomic % to 15 atomic % when measured by X-ray photoelectron spectroscopy (XPS).

The hydrogen and/or nitrogen that is contained in the carbon-based protective layer does not need to be uniformly contained in the entire carbon-based protective layer. The carbon-based protective layer is suitably made into, for example, a compositionally graded layer in which nitrogen is contained on the lubricating layer 18 side of the protective layer 17 and hydrogen is contained on the magnetic layer 16 side of the protective layer 17. In this case, the attachment forces between the magnetic layer 16 and the carbon-based protective layer and between the lubricating layer 18 and the carbon-based protective layer further improve.

The film thickness of the protective layer 17 is preferably 1 nm to 7 nm. When the film thickness of the protective layer 17 is 1 nm or more, performances as the protective layer 17 can be sufficiently obtained. The film thickness of the protective layer 17 is preferably 7 nm or less from the viewpoint of the thickness reduction of the protective layer 17.

As the film formation method of the protective layer 17, it is possible to use a sputtering method in which a carbon-containing target material is used, a CVD (chemical vapor deposition) method in which a hydrocarbon raw material, such as ethylene or toluene, is used, an IBD (ion beam deposition) method, and the like.

In the case of forming the carbon-based protective layer as the protective layer 17, the protective layer can be formed by, for example, a DC magnetron sputtering method. Particularly, in the case of forming the carbon-based protective layer as the protective layer 17, it is preferable to form an amorphous carbon protective layer by a plasma CVD method. The amorphous carbon protective layer formed by the plasma CVD method has a uniform surface and small roughness.

### Lubricating Layer

The lubricating layer 18 prevents contamination of the magnetic recording medium 10. In addition, the lubricating layer 18 reduces the friction force of a magnetic head of a magnetic recording and reproducing device that slides on the magnetic recording medium 10 to improve the durability of the magnetic recording medium 10.

The lubricating layer 18 is formed on and in contact with the protective layer 17 as shown in FIG. 1. The lubricating layer 18 contains the above-described fluorine-containing ether compound.

In a case where the protective layer 17 disposed under the lubricating layer 18 is a carbon-based protective layer, the lubricating layer 18 is particularly bonded to the protective layer 17 with a high bonding force. As a result, even when the thickness of the lubricating layer 18 is thin, it becomes easy to obtain the magnetic recording medium 10 in which the surface of the protective layer 17 is coated with a high coating rate, and contamination of the surface of the magnetic recording medium 10 can be effectively prevented.

The average film thickness of the lubricating layer 18 is preferably 0.5 nm (5 Å) to 2.0 nm (20 Å) and more preferably 0.5 nm (5 Å) to 1.0 nm (10 Å). When the average film thickness of the lubricating layer 18 is 0.5 nm or more, the lubricating layer 18 does not become an island shape or a mesh shape and is formed in a uniform film thickness. Therefore, the surface of the protective layer 17 can be coated with the lubricating layer 18 with a high coating rate. In addition, when average film thickness of the lubricating layer 18 is made to be 2.0 nm or less, it is possible to sufficiently reduce the thickness of the lubricating layer 18, and the flying height of the magnetic head can be sufficiently decreased.

In a case where the surface of the protective layer 17 is not coated with the lubricating layer 18 with a sufficiently high coating rate, an environmental substance adsorbed to the surface of the magnetic recording medium 10 passes through the gap of the lubricating layer 18 and intrudes into a layer below the lubricating layer 18. The environmental substance that has intruded into the layer below the lubricating layer 18 is adsorbed and bonded to the protective layer 17 and generates a contamination substance. In addition, at the time of magnetic recording reproduction, this contamination substance (aggregation component) is attached (transferred) to the magnetic head as a smear to break the magnetic head or degrade the magnetic recording and reproducing characteristics of the magnetic recording and reproducing device.

Examples of the environmental substance that generates the contamination substance include siloxane compounds (cyclic siloxane and linear siloxane), ionic impurities, hydrocarbons having a relatively high molecular weight, such as octacosane, plasticizers, such as dioctyl phthalate, and the like. Examples of metal ions that are contained in the ionic impurities include a sodium ion, a potassium ion, and the like. Examples of inorganic ions that are contained in the ionic impurities include a chloride ion, a bromide ion, a nitrate ion, a sulfate ion, an ammonium ion, and the like. Examples of organic substance ions that are contained in the ionic impurities include an oxalate ion, a formate ion, and the like.

### Method for Forming Lubricating Layer

Examples of a method for forming the lubricating layer 18 include methods in which a magnetic recording medium that is in the middle of production and has the individual layers up to the protective layer 17 formed on the substrate 11 is prepared, and a solution for forming the lubricating layer is applied onto the protective layer 17 and dried.

The solution for forming the lubricating layer can be obtained by dispersing and dissolving the above-described lubricant for a magnetic recording medium of the present embodiment in a solvent as necessary and adjusting the viscosity and the concentration to be suitable for an application method.

Examples of the solvent that is used in the solution for forming the lubricating layer include fluorine-based solvents, such as VERTREL (registered trademark) XF (trade name, manufactured by Du Pont-Mitsui Fluorochemicals Company, Ltd.) and/or ASAHIKLIN (registered trademark) AE-3000 (trade name, manufactured by AGC Inc.), and the like.

A method for applying the solution for forming the lubricating layer is not particularly limited, and examples thereof include a spin coating method, a spraying method, a paper coating method, a dipping method, and the like.

In the case of using the dipping method, it is possible to use, for example, a method to be described below. First, the substrate 11 on which the individual layers up to the protective layer 17 have been formed is immersed in the solution for forming the lubricating layer that has been put into an immersion tank of a dip coater. Next, the substrate 11 is lifted from the immersion tank at a predetermined rate. This makes the solution for forming the lubricating layer applied onto the surface of the protective layer 17 on the substrate 11.

The use of the dipping method makes it possible to uniformly coat the surface of the protective layer 17 with the solution for forming the lubricating layer and makes it possible to form the lubricating layer 18 on the protective layer 17 in a uniform film thickness.

In the present embodiment, a thermal treatment is preferably performed on the substrate 11 on which the lubricating layer 18 has been formed. The thermal treatment performed improves the adhesion between the lubricating layer 18 and the protective layer 17 and improves the attachment force between the lubricating layer 18 and the protective layer 17.

The thermal treatment temperature is preferably set to 100° C. to 180° C. When the thermal treatment temperature is 100° C. or higher, an effect of improving the adhesion between the lubricating layer 18 and the protective layer 17 can be sufficiently obtained. In addition, when the thermal treatment temperature is set to 180° C. or lower, the thermal decomposition of the lubricating layer 18 can be prevented. The thermal treatment time is preferably set to 10 to 120 minutes.

The magnetic recording medium 10 of the present embodiment is provided with at least the magnetic layer 16, the protective layer 17, and the lubricating layer 18 in order on the substrate 11. In the magnetic recording medium 10 of the present embodiment, the lubricating layer 18 containing the above-described fluorine-containing ether compound is formed on and in contact with the protective layer 17. This lubricating layer 18 is capable of coating the surface of the protective layer 17 with a high coating rate, has excellent adhesion to the protective layer, has favorable chemical substance resistance and wear resistance, and has excellent pickup resistance even when the thickness is thin. Therefore, the magnetic recording medium 10 of the present embodiment is capable of stably floating a magnetic head and is favorable in long-term reliability and durability.

### EXAMPLES

Hereinafter, the present invention will be more specifically described with examples and comparative examples. The present invention is not limited only to the following examples.

### Example 1

A compound represented by the formula (1A) was obtained by a method to be described below.

### First Reaction

5.0 g of a compound represented by HOCH₂CF₂O(CF₂CF₂O)ᵢ(CF₂O)ⱼCF₂CH₂OH (i indicating the average degree of polymerization in the formula was 4.5, and j indicating the average degree of polymerization was 4.5) (number-average molecular weight 997, molecular weight distribution 1.1), 1.4 g (molecular weight 137, 10.5 mmol) of 2-bromoacetamide, and 7.3 mL of N,N-dimethylacetamide (DMF) were charged into a 100 mL eggplant flask in a nitrogen gas atmosphere and stirred at room temperature to produce a mixed liquid. 4.1 g (molecular weight 325, 12.5 mmol) of cesium carbonate was added to this mixed liquid and stirred at 70° C. for eight hours to be reacted.

A reaction product obtained after the reaction was cooled to 0° C., and 50 mL of an ammonium chloride aqueous solution was added thereto to stop the reaction. The obtained reaction solution was transferred to a separatory funnel and extracted three times with 100 mL of ethyl acetate. An organic layer was washed with a saline solution and dehydrated with anhydrous sodium sulfate. After a desiccant was filtered, a filtrate was concentrated, a residue was purified with silica gel column chromatography, and 4.5 g (number-average molecular weight 1109) of a compound (1A) (in Rf₁ in the formula (1A), i indicating the average degree of polymerization was 4.5, and j indicating the average degree of polymerization was 4.5).

¹H-NMR and ¹⁹F-NMR measurement of the obtained compound (1A) was performed, and the structure was identified from the following results.

¹H-NMR (acetone-D₆): δ [ppm]=3.70 to 4.45 (8H), 6.00 to 7.50 (4H)

¹⁹F-NMR (acetone-D₆): δ [ppm]=−55.6 to −51.5 (9F), −77.5 (2F), −80.5 (2F), −91.2 to −88.4 (18F)

### Example 2

The same operation as in Example 1 was performed except that 1.9 g (molecular weight 180, 10.5 mmol) of 2-bromo-N-propylacetamide was used instead of 2-bromoacetamide in Example 1 described above, and 4.6 g (number-average molecular weight 1195) of a compound (1B) (in Rf₁ in the formula (1B), i indicating the average degree of polymerization was 4.5, and j indicating the average degree of polymerization was 4.5) was obtained.

¹H-NMR and ¹⁹F-NMR measurement of the obtained compound (1B) was performed, and the structure was identified from the following results.

¹H-NMR (acetone-D₆): δ [ppm]=0.75 to 1.20 (6H), 1.50 to 1.80 (4H), 3.00 to 3.45 (4H), 3.70 to 4.45 (8H), 6.00 to 7.50 (2H)

¹⁹F-NMR (acetone-D₆): δ [ppm]=−55.6 to −51.5 (9F), −77.5 (2F), −80.5 (2F), −91.2 to −88.4 (18F)

### Example 3

The same operation as in Example 1 was performed except that 5.0 g of a compound represented by HOCH₂CF₂O(CF₂CF₂O)ᵢ(CF₂O)ⱼCF₂CH₂OH (i indicating the average degree of polymerization in the formula was 7.1, and j indicating the average degree of polymerization was 0) (number-average molecular weight 1001, molecular weight distribution 1.1) was used instead of the compound represented by HOCH₂CF₂O(CF₂CF₂O)ᵢ(CF₂O)ⱼCF₂CH₂OH (i indicating the average degree of polymerization in the formula was 4.5, and j indicating the average degree of polymerization was 4.5) (number-average molecular weight 997, molecular weight distribution 1.1), and 1.6 g (molecular weight 152, 10.5 mmol) of 3-bromopropionamide was used instead of 2-bromoacetamide in Example 1 described above, and 4.6 g (number-average molecular weight 1143) of a compound (1C) (in Rf₁ in the formula (1C), i indicating the average degree of polymerization was 7.1 and j indicating the average degree of polymerization was 0) was obtained.

¹H-NMR and ¹⁹F-NMR measurement of the obtained compound (1C) was performed, and the structure was identified from the following results.

¹H-NMR (acetone-D₆): δ [ppm]=1.90 to 2.40 (4H), 3.55 to 4.45 (8H), 6.00 to 7.50 (4H)

¹⁹F-NMR (acetone-D₆): δ [ppm]=−78.6 (2F), −81.3 (2F), −91.0 to −88.1 (28.4F)

### Example 4

The same operation as in Example 3 was performed except that 1.9 g (molecular weight 182, 10.5 mmol) of 2-(2-bromoethoxy) acetamide was used instead of 3-bromopropionamide in Example 3 described above, and 4.7 g (number-average molecular weight 1203) of a compound (1D) (in Rf₁ of the formula (1D), i indicating the average degree of polymerization was 7.1 and j indicating the average degree of polymerization was 0) was obtained.

¹H-NMR and ¹⁹F-NMR measurement of the obtained compound (1D) was performed, and the structure was identified from the following results.

¹H-NMR (acetone-D₆): δ [ppm]=3.45 to 4.60 (16H), 6.00 to 7.50 (4H)

¹⁹F-NMR (acetone-D₆): δ [ppm]=−78.6 (2F), −81.3 (2F), −91.0 to −88.1 (28.4F)

### Example 5

The same operation as in Example 1 was performed except that 5.0 g of a compound represented by HOCH₂CF₂CF₂O(CF₂CF₂CF₂O)ₖCF₂CF₂CH₂OH (k indicating the average degree of polymerization in the formula was 4.4) (number-average molecular weight 1008, molecular weight distribution 1.1) was used instead of the compound represented by HOCH₂CF₂O(CF₂CF₂O)ᵢ(CF₂O)ⱼCF₂CH₂OH (i indicating the average degree of polymerization in the formula was 4.5, and j indicating the average degree of polymerization was 4.5) (number-average molecular weight 997, molecular weight distribution 1.1), and 3.0 g (molecular weight 287, 10.5 mmol) of an amide compound represented by the following formula (14) was used instead of 2-bromoacetamide in Example 1 described above, and 4.8 g (number-average molecular weight 1238) of a compound (1E) (in Rf₂ of the formula (1E), k indicating the average degree of polymerization was 4.4) was obtained.

The amide compound represented by the formula (14) was synthesized by reacting ethylene glycol ditosylate with 3-hydroxypropionamide.

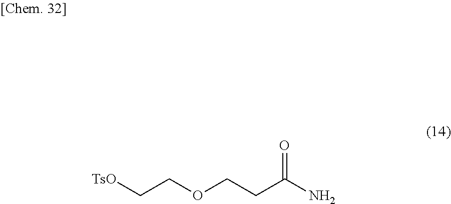 [SMILES: Cc1ccc(S(=O)(=O)OCCOCCC(N)=O)cc1]

(in the formula (14), Ts represents a p-toluenesulfonyl group.)

¹H-NMR and ¹⁹F-NMR measurement of the obtained compound (1E) was performed, and the structure was identified from the following results.

¹H-NMR (acetone-D₆): δ [ppm]=1.90 to 2.40 (4H), 3.45 to 4.65 (16H), 6.00 to 7.50 (4H)

¹⁹F-NMR (acetone-D₆): δ [ppm]=−84.3 to −82.8 (17.6F), −86.4 (4F), −124.0 (4F), −130.0 to −128.2 (8.8F)

### Example 6

A compound represented by the formula (1F) was obtained by a method to be described below.

### First Reaction

5.0 g of a compound represented by HOCH₂CF₂CF₂O(CF₂CF₂CF₂O)ₖCF₂CF₂CH₂OH (k indicating the average degree of polymerization in the formula was 4.4) (number-average molecular weight 1008, molecular weight distribution 1.1), 0.6 g (molecular weight 137, 5.0 mmol) of 2-bromoacetamide, and 7.3 mL of N,N-dimethylacetamide (DMF) were charged into a 100 mL eggplant flask in a nitrogen gas atmosphere and stirred at room temperature to produce a mixed liquid. 2.0 g (molecular weight 325, 6.2 mmol) of cesium carbonate was added to this mixed liquid and stirred at 70° C. for eight hours to be reacted.

A reaction product obtained after the reaction was cooled to 0° C., and 50 mL of an ammonium chloride aqueous solution was added thereto to stop the reaction. The obtained reaction solution was transferred to a separatory funnel and extracted three times with 100 mL of ethyl acetate. An organic layer was washed with a saline solution and dehydrated with anhydrous sodium sulfate. After a desiccant was filtered, a filtrate was concentrated, a residue was purified with silica gel column chromatography, and 4.2 g of a compound indicated by the following formula (15) was obtained as an intermediate compound (1F-1).

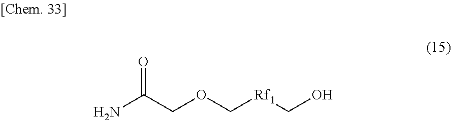

(in the formula (15), Rf₂ is represented by the above-described formula, and k indicating the average degree of polymerization in Rf₂ was 4.4.)

### Second Reaction

4.2 g (number-average molecular weight 1065) of the compound represented by the formula (15), which was the intermediate compound (1F-1), 0.6 g (molecular weight 158, 4.0 mmol) of a compound (Ep-1) represented by the following formula (16), and 5.8 mL of t-butanol were charged into a 100 mL eggplant flask in a nitrogen gas atmosphere and stirred at room temperature until the liquid became uniform. 0.13 g (molecular weight 112, 1.2 mmol) of potassium tert-butoxide was further added to this uniform liquid and stirred at 70° C. for 16 hours to be reacted.

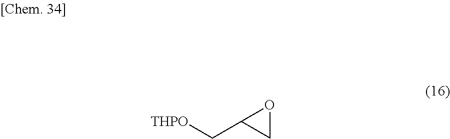

(in the formula (16), THP represents a tetrahydropyranyl group.)

A reaction solution obtained after the reaction was returned to room temperature, 20 g of a 10% hydrogen chloride-methanol solution (hydrogen chloride-methanol reagent (5% to 10%), manufactured by Tokyo Chemical Industry Co., Ltd.) was added thereto and stirred at room temperature for two hours. The reaction solution was transferred to a separatory funnel containing 100 mL of a saline solution little by little and extracted three times with 200 mL of ethyl acetate. An organic layer was washed in order with 100 mL of a saline solution, 100 mL of a saturated sodium bicarbonate solution, and 100 mL of a saline solution and dehydrated with anhydrous sodium sulfate. After a desiccant was filtered, a filtrate was concentrated, and a residue was purified with silica gel column chromatography. The above-described steps were performed, whereby 3.6 g (number-average molecular weight 1139) of a compound (1F) (in Rf₂ in the formula (1F), k indicating the average degree of polymerization was 4.4) was obtained.

¹H-NMR and ¹⁹F-NMR measurement of the obtained compound (1F) was performed, and the structure was identified from the following results.

¹H-NMR (acetone-D₆): δ [ppm]=3.25 to 4.45 (13H), 6.00 to 7.50 (2H)

¹⁹F-NMR (acetone-D₆): δ [ppm]=−84.3 to −82.8 (17.6F), −86.4 (4F), −124.0 (4F), −130.0 to −128.2 (8.8F)

### Example 7

The same operation as in Example 6 was performed except that 0.8 g (molecular weight 202, 4.0 mmol) of a compound (Ep-2) represented by the following formula (17) was used instead of the compound (Ep-1) represented by the formula (16) in the second reaction of Example 6 described above, and 3.7 g (number-average molecular weight 1183) of a compound (1G) (in Rf₂ of the formula (1G), k indicating the average degree of polymerization was 4.4) was obtained.

The compound (Ep-2) represented by the formula (17) was synthesized by a method in which a compound protecting a hydroxyl group of ethylene glycol monoallyl ether was oxidized using dihydropyran.

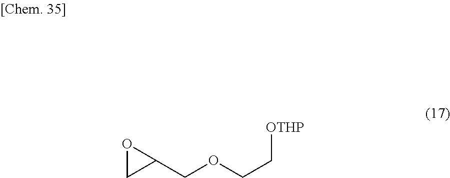

(in the formula (17), THP represents a tetrahydropyranyl group.)

¹H-NMR and ¹⁹F-NMR measurement of the obtained compound (1G) was performed, and the structure was identified from the following results.

¹H-NMR (acetone-D₆): δ [ppm]=3.25 to 4.50 (17H), 6.00 to 7.50 (2H)

¹⁹F-NMR (acetone-D₆): δ [ppm]=−84.3 to −82.8 (17.6F), −86.4 (4F), −124.0 (4F), −130.0 to −128.2 (8.8F)

### Example 8

The same operation as in Example 6 was performed except that 0.7 g (molecular weight 188, 4.0 mmol) of a compound (Ep-3) represented by the following formula (18) was used instead of the compound (Ep-1) represented by the formula (16) in the second reaction of Example 6 described above, and 3.5 g (number-average molecular weight 1124) of a compound (1H) (in Rf₂ of the formula (1H), k indicating the average degree of polymerization was 4.4) was obtained.

The compound (Ep-3) represented by the formula (18) was synthesized by reacting solketal and epibromohydrin.

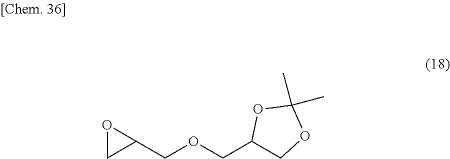

¹H-NMR and ¹⁹F-NMR measurement of the obtained compound (1H) was performed, and the structure was identified from the following results.

¹H-NMR (acetone-D₆): δ [ppm]=3.25 to 4.55 (19H), 6.00 to 7.50 (2H)

¹⁹F-NMR (acetone-D₆): δ [ppm]=−84.3 to −82.8 (17.6F), −86.4 (4F), −124.0 (4F), −130.0 to −128.2 (8.8F)

### Example 9

The same operation as in Example 6 was performed except that 1.2 g (molecular weight 320, 4.0 mmol) of a compound (Ep-4) represented by the following formula (19) was used instead of the compound (Ep-1) represented by the formula (16) in the second reaction of Example 6 described above, and 3.9 g (number-average molecular weight 1256) of a compound (11) (in Rf₂ of the formula (1I), k indicating the average degree of polymerization was 4.4) was obtained.

The compound (Ep-4) represented by the formula (19) was synthesized by the following method.

A tert-butyldimethylsilyl (TBS) group was introduced as a protective group into a primary hydroxyl group of 3-allyloxy-1,2-propane diol, and a methoxymethyl (MOM) group was introduced as a protective group into a secondary hydroxyl group of the obtained compound. The TBS group was removed from the obtained compound, and 2-bromoethoxytetrahydropyran was reacted with the generated primary hydroxyl group. A double bond of the obtained compound was oxidized. The compound (Ep-4) represented by the formula (19) was obtained by the above steps.

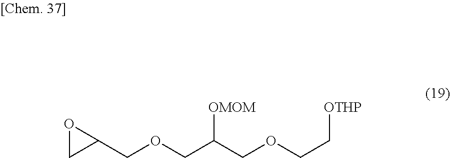

(in the formula (19), THP represents a tetrahydropyranyl group, and MOM represents a methoxymethyl group.)

¹H-NMR and ¹⁹F-NMR measurement of the obtained compound (11) was performed, and the structure was identified from the following results.

¹H-NMR (acetone-D₆): δ [ppm]=3.25 to 4.60 (23H), 6.00 to 7.60 (2H)

¹⁹F-NMR (acetone-D₆): δ [ppm]=−84.3 to −82.8 (17.6F), −86.4 (4F), −124.0 (4F), −130.0 to −128.2 (8.8F)

### Example 10

The same operation as in Example 6 was performed except that 1.2 g (molecular weight 300, 4.0 mmol) of a compound (Ep-5) represented by the following formula (20) was used instead of the compound (Ep-1) represented by the formula (16) in the second reaction of Example 6 described above, and 4.0 g (number-average molecular weight 1281) of a compound (1J) (in Rf₂ of the formula (1J), k indicating the average degree of polymerization was 4.4) was obtained.

The compound (Ep-5) represented by the following formula (20) was synthesized by a method to be described below.

Two equivalents of 3-buten-1-ol and one equivalent of epichlorohydrin were reacted with each other. 3,4-Dihydro-2H-pyran was reacted with the obtained compound to protect a secondary hydroxyl group of the compound with a tetrahydropyranyl (THP) group. A double bond on one side of the obtained compound was oxidized using m-chloroperbenzoic acid. The compound (Ep-5) represented by the formula (20) was obtained by the above steps.

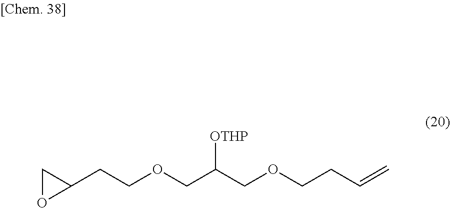

(in the formula (20), THP represents a tetrahydropyranyl group.)

¹H-NMR and ¹⁹F-NMR measurement of the obtained compound (1J) was performed, and the structure was identified from the following results.

¹H-NMR (acetone-D₆): δ [ppm]=1.50 to 1.90 (2H), 3.10 to 4.40 (22H), 4.90 to 5.40 (2H), 5.60 to 6.00 (1H), 6.00 to 7.60 (2H)

¹⁹F-NMR (acetone-D₆): δ [ppm]=−84.3 to −82.8 (17.6F), −86.4 (4F), −124.0 (4F), −130.0 to −128.2 (8.8F)

### Example 11

The same operation as in Example 1 was performed except that 1.7 g (molecular weight 166, 10.5 mmol) of N-(2-bromoethyl) acetamide was used instead of 2-bromoacetamide in Example 1 described above, and 4.5 g (number-average molecular weight 1167) of a compound (1K) (in Rf₁ in the formula (1K), i indicating the average degree of polymerization was 4.5, and j indicating the average degree of polymerization was 4.5) was obtained.

¹H-NMR and ¹⁹F-NMR measurement of the obtained compound (1K) was performed, and the structure was identified from the following results.

¹H-NMR (acetone-D₆): δ [ppm]=1.90 to 2.20 (6H), 3.70 to 4.45 (12H), 6.00 to 7.50 (2H)

¹⁹F-NMR (acetone-D₆): δ [ppm]=−55.6 to −51.5 (9F), −77.5 (2F), −80.5 (2F), −91.2 to −88.4 (18F)

### Example 12

The same operation as in Example 1 was performed except that 1.9 g (molecular weight 180, 10.5 mmol) of a compound (1L-1) represented by the following formula (21) was used instead of 2-bromoacetamide in Example 1 described above, and 4.6 g (number-average molecular weight 1195) of a compound (1L) (in Rf₁ in the formula (1L), i indicating the average degree of polymerization was 4.5, and j indicating the average degree of polymerization was 4.5) was obtained.

The compound (1L-1) represented by the following formula (21) was synthesized by reacting 2-bromoethylamine and propionic anhydride.

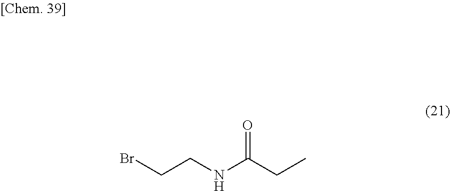

¹H-NMR and ¹⁹F-NMR measurement of the obtained compound (1L) was performed, and the structure was identified from the following results.

¹H-NMR (acetone-D₆): δ [ppm]=0.75 to 1.20 (6H), 1.90 to 2.20 (4H), 3.70 to 4.45 (12H), 6.00 to 7.50 (2H)

¹⁹F-NMR (acetone-D₆): δ [ppm]=−55.6 to −51.5 (9F), −77.5 (2F), −80.5 (2F), −91.2 to −88.4 (18F)

### Example 13

The same operation as in Example 3 was performed except that 3.2 g (molecular weight 301, 10.5 mmol) of a compound (1M-1) represented by the following formula (22) was used instead of 3-bromopropionamide in Example 3 described above, and 4.6 g (number-average molecular weight 1259) of a compound (1M) (in Rf₁ in the formula (1M), i indicating the average degree of polymerization was 7.1, and j indicating the average degree of polymerization was 0) was obtained.

The compound (1M-1) represented by the following formula (22) was synthesized by acetylating an amine portion of 2-(2-aminoethoxy) ethanol and then reacting a hydroxyl group with p-toluenesulfonyl chloride.

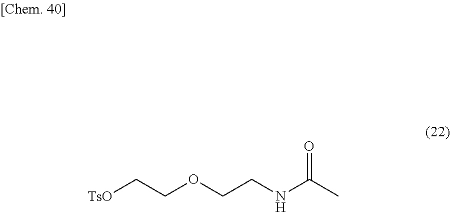 [SMILES: CC(=O)NCCOCCOS(=O)(=O)c1ccc(C)cc1]

(in the formula (22), Ts represents a p-toluenesulfonyl group.)

¹H-NMR and ¹⁹F-NMR measurement of the obtained compound (1M) was performed, and the structure was identified from the following results.

¹H-NMR (acetone-D₆): δ [ppm]=1.90 to 2.20 (6H), 3.30 to 4.55 (20H), 6.00 to 7.50 (2H)

¹⁹F-NMR (acetone-D₆): δ [ppm]=−78.6 (2F), −81.3 (2F), −91.0 to −88.1 (28.4F)

### Example 14

The same operation as in Example 3 was performed except that 3.3 g (molecular weight 315, 10.5 mmol) of a compound (1N-1) represented by the following formula (23) was used instead of 3-bromopropionamide in Example 3 described above, and 4.7 g (number-average molecular weight 1287) of a compound (1N) (in Rf₁ in the formula (1N), i indicating the average degree of polymerization was 7.1, and j indicating the average degree of polymerization was 0) was obtained.

The compound (1N-1) represented by the following formula (23) was synthesized by acetylating an amine portion of 2-(3-aminopropoxy) ethanol and then reacting a hydroxyl group with p-toluenesulfonyl chloride.

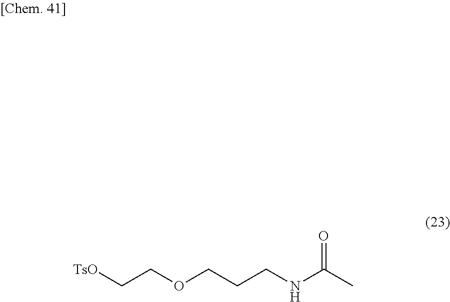 [SMILES: CC(=O)NCCCOCCOS(=O)(=O)c1ccc(C)cc1]

(in the formula (23), Ts represents a p-toluenesulfonyl group.)

¹H-NMR and ¹⁹F-NMR measurement of the obtained compound (1N) was performed, and the structure was identified from the following results.

¹H-NMR (acetone-D₆): δ [ppm]=0.75 to 1.30 (4H), 1.90 to 2.20 (6H), 3.30 to 4.55 (20H), 6.00 to 7.50 (2H)

¹⁹F-NMR (acetone-D₆): δ [ppm]=−78.6 (2F), −81.3 (2F), −91.0 to −88.1 (28.4F)

### Example 15

The same operation as in Example 5 was performed except that 3.3 g (molecular weight 315, 10.5 mmol) of a compound (10-1) represented by the following formula (24) was used instead of the amide compound represented by the formula (14) in Example 5 described above, and 4.7 g (number-average molecular weight 1294) of a compound (10) (in Rf₂ of the formula (1O), k indicating the average degree of polymerization was 4.4) was obtained.

The compound (10-1) represented by the following formula (24) was synthesized by acetylating an amine portion of 3-(2-aminoethoxy) propanol and then reacting a hydroxyl group with p-toluenesulfonyl chloride.

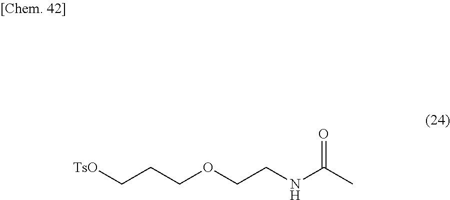 [SMILES: CC(=O)NCCOCCCOS(=O)(=O)c1ccc(C)cc1]

(in the formula (24), Ts represents a p-toluenesulfonyl group.)

¹H-NMR and ¹⁹F-NMR measurement of the obtained compound (10) was performed, and the structure was identified from the following results.

¹H-NMR (acetone-D₆): δ [ppm]=0.80 to 1.40 (4H), 1.90 to 2.20 (6H), 3.30 to 4.55 (20H), 6.00 to 7.50 (2H)

¹⁹F-NMR (acetone-D₆): δ [ppm]=−84.3 to −82.8 (17.6F), −86.4 (4F), −124.0 (4F), −130.0 to −128.2 (8.8F)

### Example 16

The same operation as in Example 6 was performed except that 0.9 g (molecular weight 166, 5.5 mmol) of N-(2-bromoethyl) acetamide was used instead of 2-bromoacetamide in the first reaction of Example 6 described above, and 3.5 g (number-average molecular weight 1167) of a compound (1P) (in Rf₂ of the formula (1P), k indicating the average degree of polymerization was 4.4) was obtained.

¹H-NMR and ¹⁹F-NMR measurement of the obtained compound (1P) was performed, and the structure was identified from the following results.

¹H-NMR (acetone-D₆): δ [ppm]=1.90 to 2.20 (3H), 3.20 to 4.45 (15H), 6.00 to 7.60 (1H)

¹⁹F-NMR (acetone-D₆): δ [ppm]=−84.3 to −82.8 (17.6F), −86.4 (4F), −124.0 (4F), −130.0 to −128.2 (8.8F)

### Example 17

The same operation as in Example 7 was performed except that 0.9 g (molecular weight 166, 5.5 mmol) of N-(2-bromoethyl) acetamide was used instead of 2-bromoacetamide in the first reaction of Example 7 described above, and 3.6 g (number-average molecular weight 1211) of a compound (1Q) (in Rf₂ of the formula (1Q), k indicating the average degree of polymerization was 4.4) was obtained.

¹H-NMR and ¹⁹F-NMR measurement of the obtained compound (1Q) was performed, and the structure was identified from the following results.

¹H-NMR (acetone-D₆): δ [ppm]=1.90 to 2.20 (3H), 3.20 to 4.50 (19H), 6.00 to 7.60 (1H) ¹⁹F-NMR (acetone-D₆): δ [ppm]=−84.3 to −82.8 (17.6F), −86.4 (4F), −124.0 (4F), −130.0 to −128.2 (8.8F)

### Example 18

The same operation as in Example 8 was performed except that 0.9 g (molecular weight 166, 5.5 mmol) of N-(2-bromoethyl) acetamide was used instead of 2-bromoacetamide in the first reaction of Example 8 described above, and 3.4 g (number-average molecular weight 1152) of a compound (1R) (in Rf₂ of the formula (1R), k indicating the average degree of polymerization was 4.4) was obtained.

¹H-NMR and ¹⁹F-NMR measurement of the obtained compound (1R) was performed, and the structure was identified from the following results.

¹H-NMR (acetone-D₆): δ [ppm]=1.90 to 2.20 (3H), 3.20 to 4.55 (21H), 6.00 to 7.60 (1H)

¹⁹F-NMR (acetone-D₆): δ [ppm]=−84.3 to −82.8 (17.6F), −86.4 (4F), −124.0 (4F), −130.0 to −128.2 (8.8F)

### Example 19

The same operation as in Example 9 was performed except that 0.9 g (molecular weight 166, 5.5 mmol) of N-(2-bromoethyl) acetamide was used instead of 2-bromoacetamide in the first reaction of Example 9 described above, and 3.8 g (number-average molecular weight 1284) of a compound (1S) (in Rf₂ of the formula (1S), k indicating the average degree of polymerization was 4.4) was obtained.

¹H-NMR and ¹⁹F-NMR measurement of the obtained compound (1S) was performed, and the structure was identified from the following results.

¹H-NMR (acetone-D₆): δ [ppm]=1.90 to 2.20 (3H), 3.20 to 4.60 (25H), 6.00 to 7.60 (1H)

¹⁹F-NMR (acetone-D₆): δ [ppm]=−84.3 to −82.8 (17.6F), −86.4 (4F), −124.0 (4F), −130.0 to −128.2 (8.8F)

### Example 20

The same operation as in Example 10 was performed except that 0.9 g (molecular weight 166, 5.5 mmol) of N-(2-bromoethyl) acetamide was used instead of 2-bromoacetamide in the first reaction of Example 10 described above, and 3.8 g (number-average molecular weight 1309) of a compound (1T) (in Rf₂ of the formula (1T), k indicating the average degree of polymerization was 4.4) was obtained.

¹H-NMR and ¹⁹F-NMR measurement of the obtained compound (1T) was performed, and the structure was identified from the following results.

¹H-NMR (acetone-D₆): δ [ppm]=1.50 to 1.85 (2H), 1.90 to 2.20 (3H), 3.10 to 4.40 (24H), 4.90 to 5.40 (2H), 5.60 to 6.00 (1H), 6.00 to 7.60 (1H)

¹⁹F-NMR (acetone-D₆): δ [ppm]=−84.3 to −82.8 (17.6F), −86.4 (4F), −124.0 (4F), −130.0 to −128.2 (8.8F)

### Example 21

### First Reaction

5.0 g of a compound represented by HOCH₂CF₂O(CF₂CF₂O)ᵢ(CF₂O)ⱼCF₂CH₂OH (i indicating the average degree of polymerization in the formula was 3.0, and j indicating the average degree of polymerization was 3.0) (number-average molecular weight 724, molecular weight distribution 1.1), 1.0 g (molecular weight 137, 7.6 mmol) of 2-bromoacetamide, and 10.1 mL of N,N-dimethylacetamide (DMF) were charged into a 100 mL eggplant flask in a nitrogen gas atmosphere and stirred at room temperature to produce a mixed liquid. 4.7 g (molecular weight 325, 14.5 mmol) of cesium carbonate was added to this mixed liquid and stirred at 70° C. for eight hours to be reacted.

A reaction product obtained after the reaction was cooled to 0° C., and 50 mL of an ammonium chloride aqueous solution was added thereto to stop the reaction. The obtained reaction solution was transferred to a separatory funnel and extracted three times with 100 mL of ethyl acetate. An organic layer was washed with a saline solution and dehydrated with anhydrous sodium sulfate. After a desiccant was filtered, a filtrate was concentrated, a residue was purified with silica gel column chromatography, and 4.0 g of a compound indicated by the following formula (25) was obtained as an intermediate compound (2A-1).

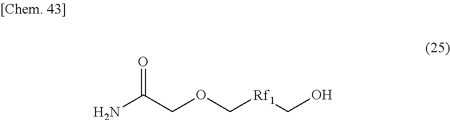

(in the formula (25), Rf₁ is represented by the formula, i indicating the average degree of polymerization in Rf₁ was 3.0, and j indicating the average degree of polymerization was 3.0.)

### Second Reaction

4.0 g (number-average molecular weight 781) of the compound represented by the formula (25), which was the intermediate compound (2A-1), 0.35 g (molecular weight 137, 2.6 mmol) of epibromohydrin, and 3.8 mL of t-butanol were charged into a 100 mL eggplant flask in a nitrogen gas atmosphere and stirred at room temperature until the liquid became uniform. 0.4 g (molecular weight 112, 4.1 mmol) of potassium tert-butoxide was further added to this uniform liquid and stirred at 70° C. for 16 hours to be reacted.

A reaction product obtained after the reaction was cooled to 0° C., and 50 mL of an ammonium chloride aqueous solution was added thereto to stop the reaction. The obtained reaction solution was transferred to a separatory funnel and extracted three times with 100 mL of ethyl acetate. An organic layer was washed with a saline solution and dehydrated with anhydrous sodium sulfate. After a desiccant was filtered, a filtrate was concentrated, and a residue was purified with silica gel column chromatography. 5.0 g (number-average molecular weight 1618) of a compound (2A) (in two Rf₁'s in the formula (2A), i indicating the average degree of polymerization was 3.0, and j indicating the average degree of polymerization was 3.0) was obtained by the above steps.

¹H-NMR and ¹⁹F-NMR measurement of the obtained compound (2A) was performed, and the structure was identified from the following results.

¹H-NMR (acetone-D₆): δ [ppm]=3.50 to 4.45 (18H), 6.00 to 7.50 (4H)

¹⁹F-NMR (acetone-D₆): δ [ppm]=−55.6 to −51.5 (12F), −77.5 (4F), −80.5 (4F), −91.2 to −88.4 (24F)

### Example 22

The same operation as in Example 21 was performed except that 5.0 g of a compound represented by HOCH₂CF₂O(CF₂CF₂O)ᵢ(CF₂O)ⱼCF₂CH₂OH (i indicating the average degree of polymerization in the formula was 5.0, and j indicating the average degree of polymerization was 0) (number-average molecular weight 758, molecular weight distribution 1.1) was used instead of the compound represented by HOCH₂CF₂O(CF₂CF₂O)ᵢ(CF₂O)ⱼCF₂CH₂OH (i indicating the average degree of polymerization in the formula was 3.0, and j indicating the average degree of polymerization was 3.0), and 1.1 g (molecular weight 152, 7.3 mmol) of 2-bromo-N-methylacetamide was used instead of 2-bromoacetamide in the first reaction of Example 21 described above, and 5.2 g (number-average molecular weight 1714) of a compound (2B) (in two Rf₁'s in the formula (2B), i indicating the average degree of polymerization was 5.0 and j indicating the average degree of polymerization was 0) was obtained.

¹H-NMR and ¹⁹F-NMR measurement of the obtained compound (2B) was performed, and the structure was identified from the following results.

¹H-NMR (acetone-D₆): δ [ppm]=2.50 to 2.95 (6H), 3.50 to 4.45 (18H), 6.00 to 7.50 (2H)

¹⁹F-NMR (acetone-D₆): δ [ppm]=−78.6 (4F), −81.3 (4F), −91.0 to −88.1 (40F)

### Example 23

The same operation as in Example 21 was performed except that 5.0 g of a compound represented by HOCH₂CF₂CF₂O(CF₂CF₂CF₂O)ₖCF₂CF₂CH₂OH (k indicating the average degree of polymerization in the formula was 3.0) (number-average molecular weight 776, molecular weight distribution 1.1) was used instead of the compound represented by HOCH₂CF₂O(CF₂CF₂O)ᵢ(CF₂O)ⱼCF₂CH₂OH (i indicating the average degree of polymerization in the formula was 3.0, and j indicating the average degree of polymerization was 3.0), and 1.1 g (molecular weight 152, 7.1 mmol) of 3-bromopropionamide was used instead of 2-bromoacetamide in the first reaction of Example 21 described above, and 5.2 g (number-average molecular weight 1750) of a compound (2C) (in two Rf₂'s in the formula (2C), k indicating the average degree of polymerization was 3.0) was obtained.

¹H-NMR and ¹⁹F-NMR measurement of the obtained compound (2C) was performed, and the structure was identified from the following results.

¹H-NMR (acetone-D₆): δ [ppm]=1.90 to 2.30 (4H), 3.50 to 4.45 (18H), 6.00 to 7.50 (4H)

¹⁹F-NMR (acetone-D₆): δ [ppm]=−84.3 to −82.8 (24F), −86.4 (8F), −124.0 (8F), −130.0 to −128.2 (12F)

### Example 24

The same operation as in Example 22 was performed except that 1.2 g (molecular weight 166, 7.3 mmol) of N-(2-bromoethyl) acetamide was used instead of 2-bromo-N-methylacetamide in the first reaction of Example 22 described above, and 5.1 g (number-average molecular weight 1742) of a compound (2D) (in two Rf₁'s in the formula (2D), i indicating the average degree of polymerization was 5.0, and j indicating the average degree of polymerization was 0) was obtained.

¹H-NMR and ¹⁹F-NMR measurement of the obtained compound (2D) was performed, and the structure was identified from the following results.

¹H-NMR (acetone-D₆): δ [ppm]=1.90 to 2.30 (6H), 3.30 to 4.45 (22H), 6.00 to 7.50 (2H)

¹⁹F-NMR (acetone-D₆): δ [ppm]=−84.3 to −82.8 (24F), −86.4 (8F), −124.0 (8F), −130.0 to −128.2 (12F)

### Example 25

The same operation as in Example 23 was performed except that 2.1 g (molecular weight 301, 7.1 mmol) of a compound (1M-1) represented by the formula (22) was used instead of 3-bromopropionamide in the first reaction of Example 23 described above, and 5.3 g (number-average molecular weight 1866) of a compound (2E) (in two Rf₂'s in the formula (2E), k indicating the average degree of polymerization was 3.0) was obtained.

¹H-NMR and ¹⁹F-NMR measurement of the obtained compound (2E) was performed, and the structure was identified from the following results.

¹H-NMR (acetone-D₆): δ [ppm]=1.90 to 2.30 (6H), 3.30 to 4.55 (30H), 6.00 to 7.50 (2H)

¹⁹F-NMR (acetone-D₆): δ [ppm]=−84.3 to −82.8 (24F), −86.4 (8F), −124.0 (8F), −130.0 to −128.2 (12F)

The value of n and the structures of R¹, R², R³, and R⁴ at the time of applying the compounds (1A) to (1T) and (2A) to (2E) of Examples 1 to 25 obtained as described above to the formula (1), respectively, are shown in Table 1 to Table 5.

TABLE 1

| | R¹ structure | R¹ | R⁴ structure | R⁴ | n | R² | R³ | Compound |
|---|---|---|---|---|---|---|---|---|
| Example 1 | 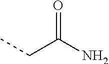 | Formula (2-1) X¹ = Formula (3) p = none, q = 0, r = 1 Y¹ = —H Z¹ = —H | 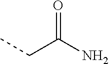 | Same as R¹ | 0 | Rf₁ i = 4.5 j = 4.5 | None | (1A) |
| Example 2 | 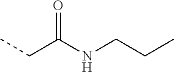 | Formula (2-1) X¹ = Formula (3) p = none, q = 0, r = 1 Y¹ = —CH₂CH₂CH₃ Z¹ = —H | 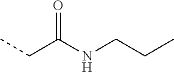 | Same as R¹ | 0 | Rf₁ i = 4.5 j = 4.5 | None | (1B) |
| Example 3 | 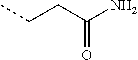 | Formula (2-1) X¹ = Formula (3) p = none, q = 0, r = 2 Y¹ = —H Z¹ = —H | 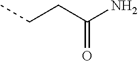 | Same as R¹ | 0 | Rf₁ i = 7.1 j = 0 | None | (1C) |
| Example 4 | 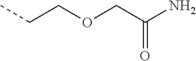 | Formula (2-1) X¹ = Formula (3) p = 2, q = 1, r = 1 Y¹ = —H Z¹ = —H | 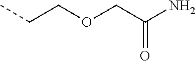 | Same as R¹ | 0 | Rf₁ i = 7.1 j = 0 | None | (1D) |
| Example 5 | 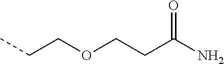 | Formula (2-1) X¹ = Formula (3) p = 2, q = 1, r = 2 Y¹ = —H Z¹ = —H | 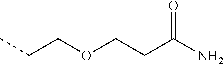 | Same as R¹ | 0 | Rf₂ k = 4.4 | None | (1E) |

TABLE 2

| | | | | | | | | Com- |
|---|---|---|---|---|---|---|---|---|
| | R¹ structure | R¹ | R⁴ structure | R⁴ | n | R² | R³ | pound |
| Ex- ample 6 | 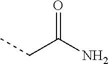 | Formula (2-1) X¹ = Formula (3) p = none, q = 0, r = 1 Y¹ = —H Z¹ = —H | 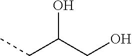 | Formula (5-3) f = none, g = 0, h = 0 Q³ = —OH | 0 | Rf₂ k = 4.4 | None | (1F) |
| Ex- ample 7 | 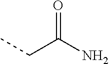 | Formula (2-1) X¹ = Formula (3) p = none, q = 0, r = 1 Y¹ = —H Z¹ = —H | 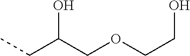 | Formula (5-1) a = 1, b = 1 Q¹ = —OH | 0 | Rf₂ k = 4.4 | None | (1G) |
| Ex- ample 8 | 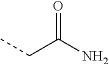 | Formula (2-1) X¹ = Formula (3) p = none, q = 0, r = 1 Y¹ = —H Z¹ = —H | 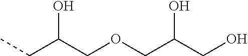 | Formula (5-3) f = 1, g = 1, h = 0 Q³ = —OH | 0 | Rf₂ k = 4.4 | None | (1H) |
| Ex- ample 9 | 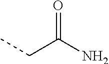 | Formula (2-1) X¹ = Formula (3) p = none, q = 0, r = 1 Y¹ = —H Z¹ = —H | 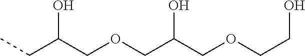 | Formula (5-1) a = 2, b = 1 Q¹ = —OH | 0 | Rf₂ k = 4.4 | None | (1I) |
| Ex- ample 10 | 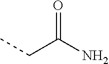 | Formula (2-1) X¹ = Formula (3) p = none, q = 0, r = 1 Y¹ = —H Z¹ = —H | 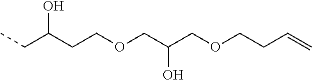 | Formula (5-2) c = 1, d = 1, e = 1 Q² = —CH═CH₂ | 0 | Rf₂ = 4.4 | None | (1J) |

TABLE 3

| | R¹ structure | R¹ | R⁴ structure | R⁴ | n | R² | R³ | Compound |
|---|---|---|---|---|---|---|---|---|
| Example 11 | 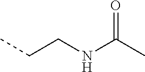 | Formula (2-2) X² = Formula (3) p = none, q = 0, r = 2 Y² = —H Z² = —CH₃ | 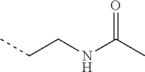 | Same as R¹ | 0 | Rf₁ i = 4.5 j = 4.5 | None | (1K) |
| Example 12 | 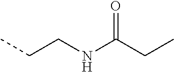 | Formula (2-2) X² = Formula (3) p = none, q = 0, r = 2 Y² = —H Z² = —CH2CH3 | 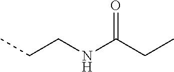 | Same as R¹ | 0 | Rf₁ i = 4.5 j = 4.5 | None | (1L) |
| Example 13 | 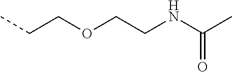 | Formula (2-2) X² = Formula (3) p = 2, q = 1, r = 2 Y² = —H Z² = —CH₃ | 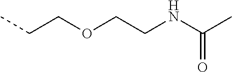 | Same as R¹ | 0 | Rf₁ i = 7.1 j = 0 | None | (1M) |
| Example 14 | 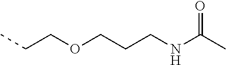 | Formula (2-2) X² = Formula (3) p = 2, q = 1, r = 3 Y² = —H Z² = —CH₃ | 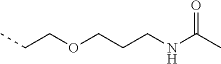 | Same as R¹ | 0 | Rf₁ i = 7.1 j = 0 | None | (1N) |
| Example 15 | 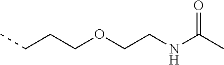 | Formula (2-2) X² = Formula (3) p = 3, q = 1, r = 2 Y² = —H Z² = —CH₃ | 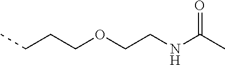 | Same as R¹ | 0 | Rf₂ k = 4.4 | None | (1O) |

TABLE 4

| | | | | | | | | Com- |
|---|---|---|---|---|---|---|---|---|
| | R¹ structure | R¹ | R⁴ structure | R⁴ | n | R² | R³ | pound |
| Example 16 | 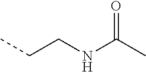 | Formula (2-2) X² = Formula (3) p = none, q = 0, r = 2 Y² = —H Z² = —CH₃ | 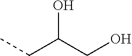 | Formula (5-3) f = none, g = 0, h = 0 Q³ = —OH | 0 | Rf₂ k = 4.4 | None | (1P) |
| Example 17 | 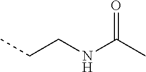 | Formula (2-2) X² = Formula (3) p = none, q = 0, r = 2 Y² = —H Z² = —CH₃ | 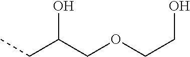 | Formula (5-1) a = 1, b = 1 Q¹ = —OH | 0 | Rf₂ k = 4.4 | None | (1Q) |
| Example 18 | 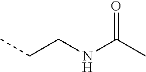 | Formula (2-2) X² = Formula (3) p = none, q = 0, r = 2 Y² = —H Z² = —CH₃ | 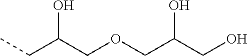 | Formula (5-3) f = 1, g = 1, h = 0 Q³ = —OH | 0 | Rf₂ k = 4.4 | None | (1R) |
| Example 19 | 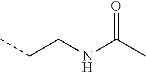 | Formula (2-2) X² = Formula (3) p = none, q = 0, r = 2 Y² = —H Z² = —CH₃ | 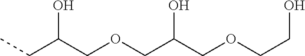 | Formula (5-1) a = 2, b = 1 Q¹ = —OH | 0 | Rf₂ k = 4.4 | None | (1S) |
| Example 20 | 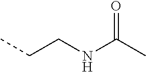 | Formula (2-2) X² = Formula (3) p = none, q = 0, r = 2 Y² = —H Z² = —CH₃ | 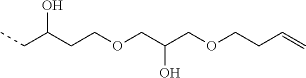 | Formula (5-2) c = 1, d = 1, e = 1 Q² = —CH═CH₂ | 0 | Rf₂ k = 4.4 | none | (1T) |

TABLE 5

| | R¹ structure | R¹ | R⁴ structure | R⁴ | n | R² | R³ | Compound |
|---|---|---|---|---|---|---|---|---|
| Example 21 | 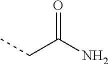 | Formula (2-1) X¹ = Formula (3) p = none, q = 0, r = 1 Y¹ = —H Z¹ = —H | 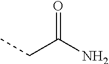 | Same as R¹ | 1 | Rf₁ i = 3.0 j = 3.0 | Formula (4) s = 1, t = 1 | (2A) |
| Example 22 | 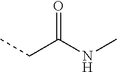 | Formula (2-1) X¹ = Formula (3) p = none, q = 0, r = 1 Y¹ = —CH₃ _{Z}¹ = —H | 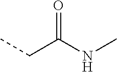 | Same as R₁ | 1 | Rf₁ i = 5.0 j = 0 | Formula (4) s = 1, t = 1 | (2B) |
| Example 23 | 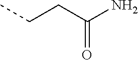 | Formula (2-1) X¹ = Formula (3) p = none, q = 0, r = 2 Y¹ = —H Z¹ = —H | 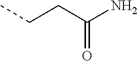 | Same as R¹ | 1 | Rf₂ k = 3.0 | Formula (4) s = 1, t = 1 | (2C) |
| Example 24 | 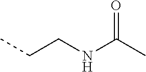 | Formula (2-2) X² = Formula (3) p = none, q = 0, r = 2 Y² = —H Z² = —CH₃ | 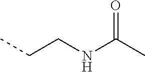 | Same as R¹ | 1 | Rf₁ i = 5.0 j = 0 | Formula (4) s = 1, t = 1 | (2D) |
| Example 25 | 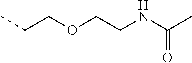 | Formula (2-2) X² = Formula (3) p = 2, q = 1, r = 2 Y² = —H Z² = —CH₃ | 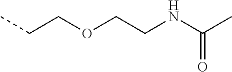 | Same as R¹ | 1 | Rf₂ k = 3.0 | Formula (4) s = 1, t = 1 | (2E) |

### Comparative Example 1

A compound (3A) represented by the following formula (26) was synthesized by a method described in Patent Document 1.

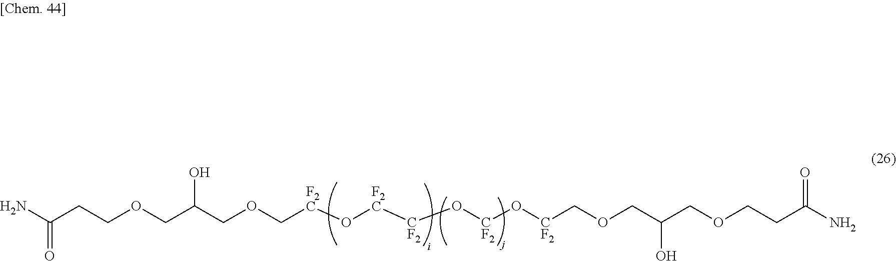

(in the formula (26), i indicating the average degree of polymerization represents 4.5, and j represents 4.5.)

### Comparative Example 2

A compound (3B) represented by the following formula (27) was synthesized by a method described in Patent Document 2.

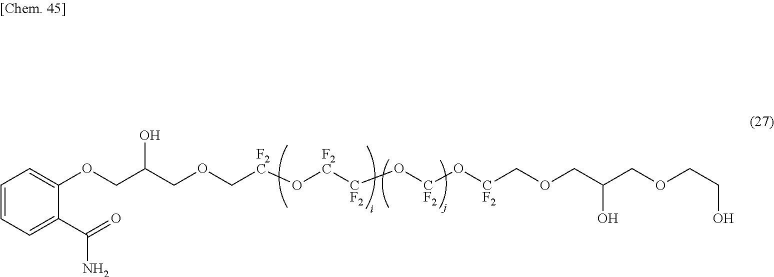

(in the formula (27), i indicating the average degree of polymerization represents 6.2, and j represents 6.2.)

### Comparative Example 3

A compound (3C) represented by the following formula (28) was synthesized with reference to a method described in Patent Document 3. In more detail, with reference to a method described in Example 1 of Patent Document 3, the compound (3C) was produced using 2-(2-aminoethoxy) ethanol instead of 3-amino-1,2-propanediol in Example 1 of Patent Document 3.

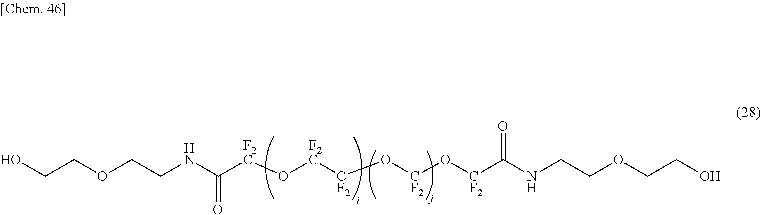

(in the formula (28), i indicating the average degree of polymerization represents 4.5, and j represents 4.5.)

### Comparative Example 4

A compound (3D) represented by the following formula (29) was synthesized by a method described in Patent Document 4.

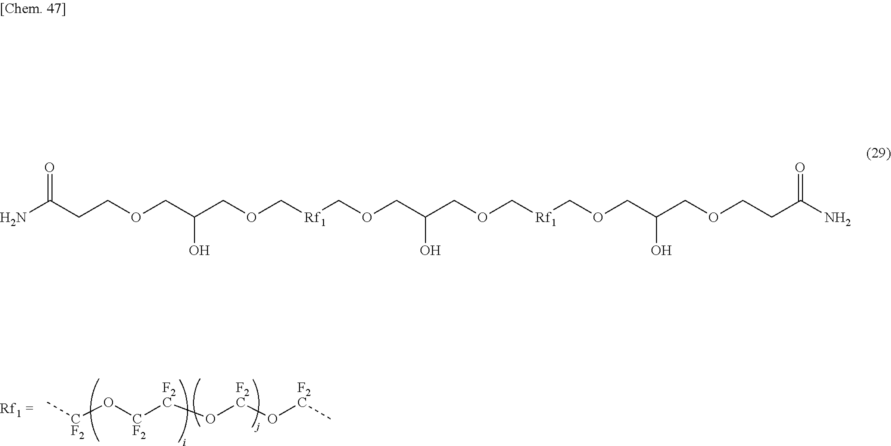

(in Rf₁ in the formula (29), i indicating the average degree of polymerization represents 3.2, and j represents 3.2.)

### Comparative Example 5

A compound (3E) represented by the following formula (30) was synthesized by the method described in Patent Document 4.

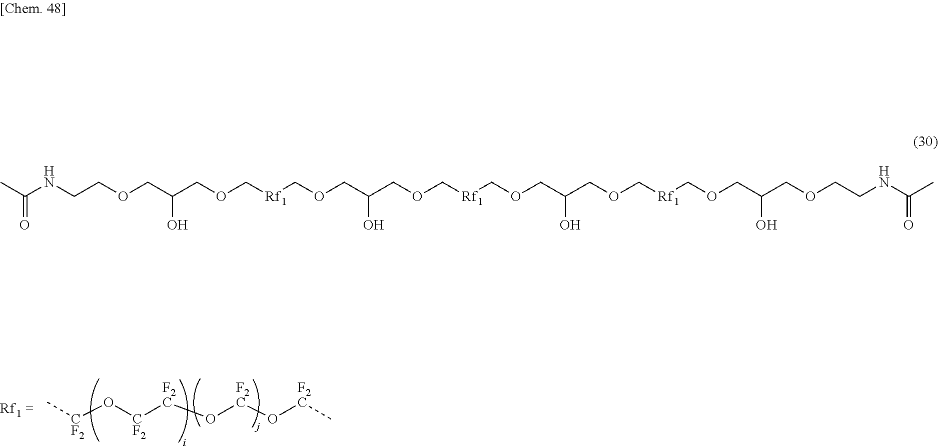

(in Rf₁ in the formula (30), i indicating the average degree of polymerization represents 3.2, and j represents 3.2.)

### Comparative Example 6

A compound (3F) represented by the following formula (31) was synthesized by the following method.

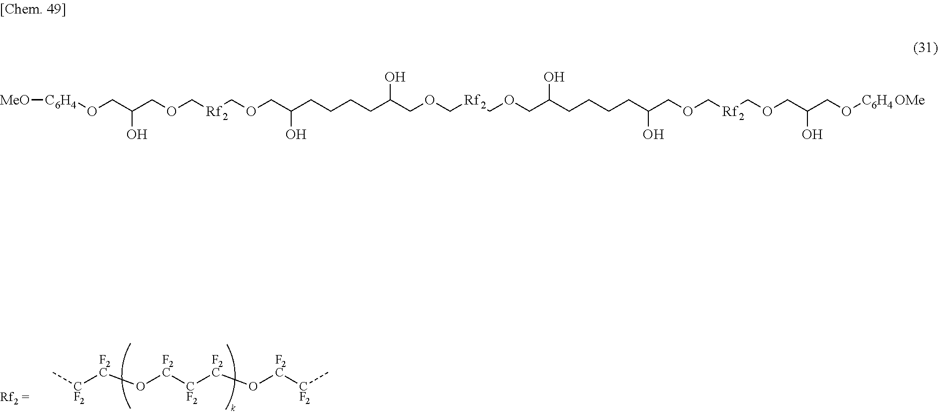

(in Rf₂ in the formula (31), k indicating the average degree of polymerization represents 4.0.)

That is, a fluorine-based compound represented by HOCH₂CF₂CF₂O(CF₂CF₂CF₂O)ₖCF₂CF₂CH₂OH (k indicating the average degree of polymerization in the formula was 4.0), 1,7-octadiepoxide, and t-butanol were charged under a nitrogen atmosphere and stirred at room temperature until the liquid became uniform. Potassium tert-butoxide was further added to this uniform liquid and stirred at 70° C. for 16 hours to be reacted. At this time, the reaction ratio between the fluorine-based compound and 1,7-octadiepoxide was set to approximately 1:2 (mole ratio). As a result, a first intermediate compound having a secondary hydroxyl group and an epoxy group at both terminals of Rf₂ was obtained.

Next, the same operation as in the production of the first intermediate compound was performed except that 4-methoxyphenyl glycidyl ether was used instead of 1,7-octadiepoxide and the reaction ratio between the fluorine-based compound and 4-methoxyphenyl glycidyl ether was set to approximately 1:1 (mole ratio) in the production of the first intermediate compound, and a second intermediate compound having a group containing 4-methoxyphenyl bonding to one terminal of Rf₂ was obtained.

Finally, the first intermediate compound and the second intermediate compound were reacted with each other in t-butanol using potassium tert-butoxide, thereby synthesizing the compound (3F).

The number-average molecular weights (Mn) of the compounds of Examples 1 to 25 and Comparative Examples 1 to 6 obtained as described above were obtained from the above-described ¹H-NMR and ¹⁹F-NMR measurement results. The results are shown in Table 6 and Table 7. It is estimated that there is a variation of approximately 1 to 5 in the values of the average molecular weights of the synthesized compounds due to the molecular weight distribution of fluoropolyether used as a raw material of the compounds, a difference in operation at the time of synthesizing the compounds, or the like.

TABLE 6

| | | Number- | | | | | |
|---|---|---|---|---|---|---|---|
| | | average | Film | Wear | Pickup | Chemical | |
| | | molecular | thickness | resistance | characteristics | resistance | Comprehensive |
| | Compound | weight (Mn) | (Å) | test | test | test | evaluation |
| Example 1 | (1A) | 1109 | 9.5 | A | B | A | B |
| Example 2 | (1B) | 1195 | 9.5 | A | B | A | B |
| Example 3 | (1C) | 1143 | 9.4 | A | B | A | B |
| Example 4 | (1D) | 1203 | 9.5 | A | A | A | A |
| Example 5 | (1E) | 1238 | 9.4 | A | A | A | A |
| Example 6 | (1F) | 1139 | 9.4 | A | B | A | B |
| Example 7 | (1G) | 1183 | 9.6 | A | A | A | A |
| Example 8 | (1H) | 1124 | 9.4 | A | A | B | B |
| Example 9 | (1I) | 1256 | 9.4 | A | B | A | B |
| Example 10 | (1J) | 1281 | 9.5 | B | A | A | B |
| Example 11 | (1K) | 1167 | 9.3 | B | B | A | B |
| Example 12 | (1L) | 1195 | 9.6 | B | B | B | B |
| Example 13 | (1M) | 1259 | 9.5 | A | A | A | A |
| Example 14 | (1N) | 1287 | 9.5 | A | A | A | A |
| Example 15 | (1O) | 1294 | 9.5 | A | A | A | A |
| Example 16 | (1P) | 1167 | 9.5 | A | B | A | B |
| Example 17 | (1Q) | 1211 | 9.4 | A | A | A | A |
| Example 18 | (1R) | 1152 | 9.5 | A | A | B | B |
| Example 19 | (1S) | 1284 | 9.4 | B | B | A | B |
| Example 20 | (1T) | 1309 | 9.4 | A | A | B | B |
| Example 21 | (2A) | 1618 | 9.4 | A | A | A | A |
| Example 22 | (2B) | 1714 | 9.4 | A | B | A | B |
| Example 23 | (2C) | 1750 | 9.4 | B | A | A | B |
| Example 24 | (2D) | 1742 | 9.4 | A | A | A | A |
| Example 25 | (2E) | 1866 | 9.4 | A | A | A | A |

TABLE 7

| | | Number- | | | | | |
|---|---|---|---|---|---|---|---|
| | | average | Film | Wear | Pickup | Chemical | |
| | | molecular | thickness | resistance | characteristics | resistance | Comprehensive |
| | Compound | weight (Mn) | (Å) | test | test | test | evaluation |
| Comparative | (3A) | 1198 | 9.4 | B | D | C | D |
| Example 1 | | | | | | | |
| Comparative | (3B) | 1611 | 9.4 | C | B | C | C |
| Example 2 | | | | | | | |
| Comparative | (3C) | 1236 | 9.3 | D | D | D | D |
| Example 3 | | | | | | | |
| Comparative | (3D) | 2380 | 9.4 | C | C | C | C |
| Example 4 | | | | | | | |
| Comparative | (3E) | 3493 | 9.3 | C | C | C | C |
| Example 5 | | | | | | | |
| Comparative | (3F) | 3660 | 9.3 | D | D | D | D |
| Example 6 | | | | | | | |

Next, solutions for forming a lubricating layer were prepared using the compounds obtained in Examples 1 to 25 and Comparative Examples 1 to 6 by a method to be described below. In addition, lubricating layers for magnetic recording media were formed by a method to be described below using the obtained solutions for forming a lubricating layer, and magnetic recording media of Examples 1 to 25 and Comparative Examples 1 to 6 were obtained.

### Solutions for Forming Lubricating Layer

Fluorine-containing ether compounds obtained in Examples 1 to 25 and Comparative Examples 1 to 6 were each dissolved in VERTREL (registered trademark) XF (trade name, manufactured by Du Pont-Mitsui Fluorochemicals Company, Ltd.), which is a fluorine-based solvent, and were diluted with VERTREL XF so that the film thickness reached 9 Å to 10 Å when the fluorine-containing ether compound was applied onto a protective layer, thereby producing the solutions for forming a lubricating layer.

### Magnetic Recording Media

Magnetic recording media each having an adhesive layer, a soft magnetic layer, a first base layer, a second base layer, a magnetic layer, and a protective layer provided in order on a substrate having a diameter of 65 mm were prepared. The protective layers were made of carbon.

The solutions for forming a lubricating layer of Examples 1 to 25 and Comparative Examples 1 to 6 were each applied onto the protective layer of the magnetic recording medium in which the individual layers up to the protective layer had been formed by a dipping method. The dipping method was performed under conditions of an immersion rate of 10 mm/sec, an immersion time of 30 sec, and a lifting rate of 1.2 mm/sec.

After that, the magnetic recording medium onto which the solution for forming a lubricating layer had been applied was put into a thermostatic chamber at 120° C. and heated for 10 minutes to remove the solvent in the solution for forming a lubricating layer, thereby forming a lubricating layer on the protective layer and obtaining a magnetic recording medium.

### Film Thickness Measurement

Regarding the lubricating layers of the magnetic recording media of Examples 1 to 25 and Comparative Examples 1 to 6 obtained as described above, peak heights at C-F vibration stretching were measured using FT-IR (trade name: Nicolet iS50, manufactured by Thermo Fisher Scientific Inc.). Next, the film thicknesses of the lubricating layers were calculated from the measurement values of the peak heights at C-F vibration stretching of the lubricating layers using a correlation formula obtained by a method to be described below. The results are shown in Table 6 and Table 7.

### Method for Calculating Correlation Formula

Discs having an adhesive layer, a soft magnetic layer, a first base layer, a second base layer, a magnetic layer, and a protective layer provided in order on a substrate having a diameter of 65 mm were prepared. Lubricating layers were formed in film thicknesses of 6 Å to 20 Å (2 Å notch) on the protective layers of these discs, respectively.

After that, regarding each disc having the lubricating layer formed thereon, an increase in the film thickness from the disc surface on which the lubricating layer was not formed was measured using an ellipsometer and was regarded as the film thickness of the lubricating layer. In addition, regarding each disc having the lubricating layer formed thereon, a peak height at C-F vibration stretching was measured using FT-IR.

In addition, a correlation formula between the peak height obtained by FT-IR and the film thickness of the lubricating layer obtained using the ellipsometer was obtained.

Next, regarding the magnetic recording media of Examples 1 to 25 and Comparative Examples 1 to 6, the wear resistance, the pickup characteristics, and the chemical substance resistance were tested and evaluated by methods to be described below. The results are shown in Table 6 and Table 7.

### Wear Resistance Test

An alumina ball having a diameter of 2 mm was slid as a contactor on a lubricating layer of a magnetic recording medium using a pin-on-disk type friction and wear tester under a load of 40 gf and at a sliding speed of 0.25 m/sec, and the friction coefficient of the surface of the lubricating layer was measured. In addition, the sliding time taken until the friction coefficient of the surface of the lubricating layer abruptly increased was measured. The sliding time taken until the friction coefficient abruptly increased was measured four times for the lubricating layer of each magnetic recording medium, and the average value (hours) was regarded as an index of the wear resistance of a lubricant coating film.

### Evaluation Standards of Wear Resistance

The wear resistance was evaluated by the sliding time taken until the friction coefficient abruptly increased as described below.
A (Excellent): 670 sec or longer
B (Favorable): 570 sec or longer and shorter than 670 sec
C (Acceptable): 450 sec or longer and shorter than 570 sec
D (Unacceptable): Shorter than 450 sec

The time taken until the friction coefficient abruptly increases can be used as an index of the wear resistance of the lubricating layer for the following reason. That is, as the magnetic recording medium is used, the lubricating layer of the magnetic recording medium undergoes wear, when the lubricating layer disappears due to the wear, the contactor and the protective layer come into direct contact with each other, and the friction coefficient abruptly increases. The time taken until the friction coefficient abruptly increases can also be considered to correlate with a friction test.

### Pickup Characteristic Test

The magnetic recording medium and a magnetic head were mounted in a spin stand and rotated at room temperature under reduced pressure (approximately 250 torr), thereby making the magnetic head float at a fixed point for 10 minutes. After that, the surface of the magnetic head facing the magnetic recording medium was analyzed using an ESCA (electron Spectroscopy for Chemical Analysis) analyzer. The intensity (signal intensity (a. u.)) of a fluorine-derived peak obtained by the analysis using the ESCA analyzer indicates the amount of the lubricant attached to the magnetic head. The pickup characteristics were evaluated by evaluation standards to be described below using the obtained signal intensity.

### Evaluation Standards of Pickup Characteristics

A (Excellent): The signal intensity is less than 150 (the attachment amount is extremely small)
B (Favorable): The signal intensity is 150 or more and less than 280 (the attachment amount is small)
C (Acceptable): The signal intensity is 280 or more and less than 1000 (the attachment amount is large)
D (Unacceptable): The signal intensity is 1000 or more (the attachment amount is extremely large)

### Chemical Resistance Test

Contamination of a magnetic recording medium by an environmental substance that generated a contamination substance under a high-temperature environment was examined by a method to be described below. Si ions were used as the environmental substance, and the amount of Si adsorbed was measured as the amount of the contamination substance that was generated by the environmental substance and contaminated the magnetic recording medium.

Specifically, a magnetic recording medium, which was an evaluation subject, was held for 240 hours under a high-temperature environment with a temperature of 85° C. and a humidity of 0% in the presence of siloxane-based Si rubber. Next, the amount of Si adsorbed present on the surface of the magnetic recording medium was analyzed and measured by secondary-ion mass spectrometry (SIMS), and the degree of contamination caused by the Si ions was evaluated as the amount of Si adsorbed. The evaluation of the amount of Si adsorbed was evaluated as described below using a numerical value obtained when the result of Comparative Example 3 was regarded as 1.00.

### Evaluation Standards of Chemical Substance Resistance

A (Excellent): Less than 0.60
B (Favorable): 0.60 or more and less than 0.75
C (Acceptable): 0.75 or more and less than 1.00
D (Unacceptable): 1.00 or more

### Comprehensive Evaluation

Comprehensive evaluation was performed from the results of the wear resistance test, the pickup characteristic test, and the chemical resistance test by evaluation standards to be described below. The results are shown in Table 6 and Table 7.

### Evaluation Standards of Comprehensive Evaluation

A: The wear resistance test, the pickup characteristic test, and the chemical resistance test are all A.
B: The evaluations of the wear resistance test, the pickup characteristic test, and the chemical resistance test are A or B, and one or more thereof are B.
C: One or more of the evaluations of the wear resistance test, the pickup characteristic test, and the chemical resistance test are C, and there is no D.
D: One or more of the evaluations of the wear resistance test, the pickup characteristic test, and the chemical resistance test are D.

As shown in Table 6, for the magnetic recording media of Examples 1 to 25, the evaluations of the wear resistance test, the pickup characteristic test, and the chemical resistance test were all “A” or “B,” and the results of the comprehensive evaluations were “A” or “B.” From these facts, it was possible to confirm that the magnetic recording media of Examples 1 to 25 had excellent wear resistance and a high pickup-curbing effect and provided high chemical substance resistance to the magnetic recording media.

In contrast, as shown in Table 7, for all of the magnetic recording media of Comparative Examples 1 to 6 having a lubricating layer formed using any of the compounds (3A) to (3F), the evaluations of the wear resistance test, the pickup characteristic test, and the chemical resistance test were B, C, or D, and the evaluation of at least one test item was C or D, which were poor compared with the magnetic recording media of Examples 1 to 25. This is estimated to be because in the magnetic recording media of Comparative Examples 1 to 6, the lubricating layers were not formed using the fluorine-containing ether compound represented by the formula (1).

In more detail, the magnetic recording media of Comparative Examples 1 to 3 had lubricating layers formed using the compounds (3A) to (3C), respectively. The compounds (3A) to (3C) all have one perfluoropolyether chain and have an amide bond portion in at least one terminal group.

The compounds (3A) and (3B) each have a divalent linking group including a secondary hydroxyl group disposed between a perfluoropolyether chain and an amide bond portion. Since the surface free energy of the entire compound increases due to the secondary hydroxyl group that was included in this linking group, it is estimated that in Comparative Examples 1 and 2, the results of the evaluation of the chemical substance resistance were poor compared with those in Examples 1 to 25. Particularly, in the lubricating layer of Comparative Example 1 formed using the compound (3A), the results of the evaluations of the pickup resistance and the chemical substance resistance were poor compared with those in Examples 1 to 25. In addition, in the compound (3B), the divalent linking group has an aromatic ring and is a highly flat terminal group to which the aromatic ring and the amide bond portion have directly bonded. Therefore, the adhesion of the amide bond portion and the aromatic ring to the protective layer is too strong, and it is estimated that in the lubricating layer of Comparative Example 2 formed using the compound (3B), the lubricity was impaired and the wear resistance was insufficient.

In the compound (3C), a carbon atom of —CF₂— that forms the perfluoropolyether chain and a carbon atom of the amide bond portion (C(═O)N) directly bond to each other with no linking groups therebetween. Therefore, the bond of the amide bond portion to the active point on the protective layer is impaired by the bulkiness of the perfluoropolyether chain. Therefore, an amide group that is not easy to freely rotate is directly bonded to the perfluoropolyether chain, whereby the mobility (degree of freedom) of the amide bond portion is considered to be low. Therefore, it is estimated that in the lubricating layer of Comparative Example 3 containing the compound (3C), the adhesion to the protective layer is insufficient and the wear resistance, the pickup resistance, and the chemical substance resistance are all insufficient.

The magnetic recording media of Comparative Examples 4 to 6 had lubricating layers formed using the compounds (3D) to (3F), respectively.

The compounds (3D) and (3E) each have a skeleton in which two or three perfluoropolyether chains bond to one another through a linking group including a secondary hydroxyl group. The compounds (3D) and (3E) each have an amide bond portion as a polar group at the terminal of this skeleton and have a divalent linking group including a secondary hydroxyl group disposed between the perfluoropolyether chain and the amide bond portion. In the lubricating layers of Comparative Examples 4 and 5 formed using the compounds (3D) and (3E), since the adsorption of the secondary hydroxyl group that is included in this divalent linking group to the protective layer is too strong, it is estimated that the lubricity was impaired and the wear resistance was insufficient. Furthermore, since the surface free energy of the entire compound became too high due to the secondary hydroxyl group that was included in the divalent linking group, it is estimated that in the lubricating layers of Comparative Examples 4 and 5, the results of the evaluations of the chemical substance resistance and the pickup resistance were poor compared with those in Examples 1 to 25.

The compound (3F) has a skeleton in which three perfluoropolyether chains have bonded to each other through a linking group including a secondary hydroxyl group. The compound (3F) has no amide bond portions at both terminals of this skeleton, has an aromatic ring, and has a divalent linking group including a secondary hydroxyl group disposed between the perfluoropolyether chain and an aromatic ring. Therefore, it is estimated that in the lubricating layer of Comparative Example 6 formed using the compound (3F), adsorption to the active point on the protective layer was insufficient and the results of the evaluations of the wear resistance, the pickup resistance, and the chemical substance resistance were poor compared with those in Examples 1 to 25.

### INDUSTRIAL APPLICABILITY

The use of a lubricant for a magnetic recording medium containing the fluorine-containing ether compound of the present invention makes it possible to form a lubricating layer having excellent chemical substance resistance, favorable wear resistance, and a high pickup-curbing effect even when the thickness is thin.

### REFERENCE SIGNS LIST

10 Magnetic recording medium
11 Substrate
12 Adhesive layer
13 Soft magnetic layer
14 First base layer
15 Second base layer
16 Magnetic layer
17 Protective layer
18 Lubricating layer

1. A fluorine-containing ether compound that is represented by a following formula (1): 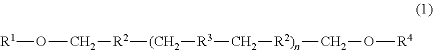
(in the formula (1), R¹ is represented by a formula (2-1) or a formula (2-2); R² is a perfluoropolyether chain; R⁴ is an organic group having at least one polar group and having 1 to 50 carbon atoms; R³ is a divalent linking group having at least one hydroxyl group; n is an integer of 0 to 2; in a case where n is 1 or 2, a part or all of a plurality of R²'s may be identical to each other or may be different from each other; and in a case where n is 2, two R³'s may be identical to each other or may be different from each other) 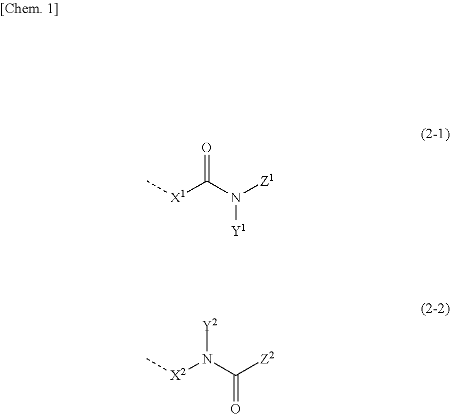
(in the formula (2-1), X¹ is a divalent linking group including no polar groups, optionally including an ether oxygen atom, and having 1 to 12 carbon atoms; Y¹ and Z¹ are each independently an organic group or a hydrogen atom, the organic group having 1 to 30 carbon atoms and optionally including at least one of a polar group and an ether oxygen atom; and Y¹ and Z¹ may bond to each other to form a ring)
(in the formula (2-2), X² is a divalent linking group including no polar groups, optionally including an ether oxygen atom, and having 1 to 12 carbon atoms; Y² is an organic group or a hydrogen atom, the organic group having 1 to 30 carbon atoms and optionally including at least one of a polar group and an ether oxygen atom; Z² is an organic group optionally including at least one of a polar group and an ether oxygen atom and having 1 to 30 carbon atoms; and Y² and Z² may bond to each other to form a ring).

2. The fluorine-containing ether compound according to claim 1, wherein X¹ in the formula (2-1) and X² in the formula (2-2) are each independently represented by a following formula (3): 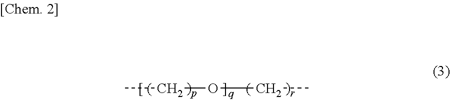
(in the formula (3), p is an integer of 2 to 4, q is an integer of 0 to 2, and r is an integer of 1 to 4; in a case where q is 2, two p's may be identical to each other or may be different from each other; and a left terminal in the formula (3) is a side bonding to an oxygen atom of —R²—CH₂—O— and a right terminal is a side bonding to a carbonyl carbon atom or nitrogen atom that configures an amide bond).

3. The fluorine-containing ether compound according to claim 1, wherein at least one of Y¹ and Z¹ in the formula (2-1) is a hydrogen atom, and Y² in the formula (2-2) is a hydrogen atom.

4. The fluorine-containing ether compound according to claim 1, wherein R³ in the formula (1) is a divalent linking group represented by a following formula (4): 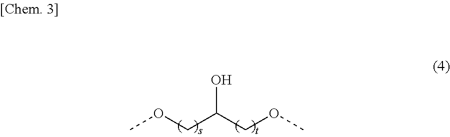
(in the formula (4), s is an integer of 1 to 3, and t is an integer of 1 to 3; and an oxygen atom at a left terminal in the formula (4) bonds to —CH₂— on an R¹ side and an oxygen atom at a right terminal bonds to —CH₂— on an R⁴ side).

5. The fluorine-containing ether compound according to claim 1, wherein R⁴ in the formula (1) is not a group represented by the formula (2-1) or the formula (2-2), and at least one of the polar groups included in R⁴ is a hydroxyl group.

6. The fluorine-containing ether compound according to claim 5, wherein R⁴ in the formula (1) includes two or three polar groups, and at least one of the polar groups is a secondary hydroxyl group.

7. The fluorine-containing ether compound according to claim 5, wherein R⁴ in the formula (1) is any of groups represented by following formulae (5-1) to (5-3): 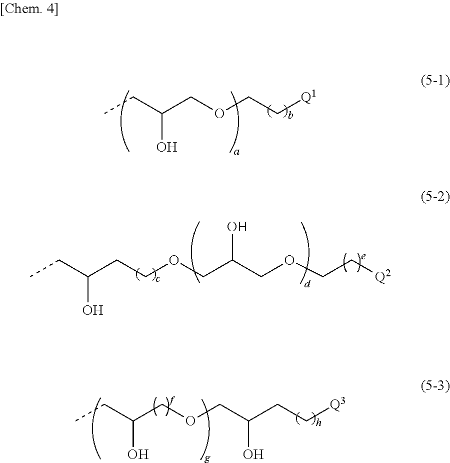
(in the formula (5-1), a is an integer of 1 or 2; b is an integer of 0 to 3; Q¹ is an aromatic hydrocarbon group, an unsaturated heterocyclic group, an alkenyl group, an alkynyl group, or a polar group; in a case where a is 1, Q¹ is a polar group; in a case where Q¹ is an aromatic hydrocarbon group or an unsaturated heterocyclic group, an atom that configures a ring structure in Q¹ bonds to a methylene group adjacent to Q¹; and in a case where Q¹ is an alkenyl group or an alkynyl group, a carbon atom that configures an unsaturated bond in Q¹ bonds to the methylene group adjacent to Q¹),
(in the formula (5-2), c is an integer of 1 to 3; d is an integer of 0 or 1; e is an integer of 0 to 3; Q² is an aromatic hydrocarbon group, an unsaturated heterocyclic group, an alkenyl group, an alkynyl group, or a polar group; in a case where d is 0, Q² is a polar group; in a case where Q² is an aromatic hydrocarbon group or an unsaturated heterocyclic group, an atom that configures a ring structure in Q² bonds to a methylene group adjacent to Q²; and in a case where Q² is an alkenyl group or an alkynyl group, a carbon atom that configures an unsaturated bond in Q² bonds to the methylene group adjacent to Q²), and
(in the formula (5-3), f is an integer of 1 to 3; g is an integer of 0 or 1; h is an integer of 0 to 3; Q³ is an aromatic hydrocarbon group, an unsaturated heterocyclic group, an alkenyl group, an alkynyl group, or a polar group; in a case where g is 0, Q³ is a polar group; in a case where Q³ is an aromatic hydrocarbon group or an unsaturated heterocyclic group; an atom that configures a ring structure in Q³ bonds to a methylene group adjacent to Q³; and in a case where Q³ is an alkenyl group or an alkynyl group, a carbon atom that configures an unsaturated bond in Q³ bonds to the methylene group adjacent to Q³).

8. The fluorine-containing ether compound according to claim 1, wherein R⁴ in the formula (1) is represented by the formula (2-1) or (2-2).

9. The fluorine-containing ether compound according to claim 8, wherein R¹ and R⁴ in the formula (1) are identical to each other.

10. The fluorine-containing ether compound according to claim 1, wherein R²'s in the formula (1) are each independently a perfluoropolyether chain represented by the following formula (6): 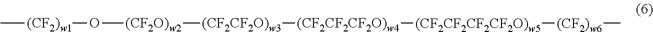
(in the formula (6), w2, w3, w4, and w5 indicate average degrees of polymerization and each independently represent 0 to 20; where w2, w3, w4, and w5 do not all become 0 at the same time; w1 and w6 are average values representing the numbers of CF₂ and each independently represent 1 to 3; and an array order of (CF₂O), (CF₂CF₂O), (CF₂CF₂CF₂O), and (CF₂CF₂CF₂CF₂O), which are repeating units in the formula (6), is not particularly limited).

11. The fluorine-containing ether compound according to claim 1, wherein R² in the formula (1) is any one selected from perfluoropolyether chains represented by following formulae (6-1) to (6-4): 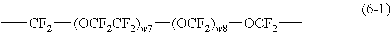
(in the formula (6-1), w7 and w8 indicate average degrees of polymerization, w7 represents 1 to 20, and w8 represents 0 to 20), 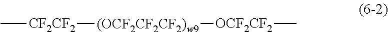
(in the formula (6-2), w9 indicates an average degree of polymerization and represents 1 to 15), 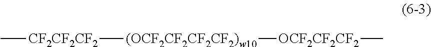
(in the formula (6-3), w10 indicates an average degree of polymerization and represents 1 to 10), and 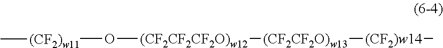
(in the formula (6-4), w12 and w13 indicate average degrees of polymerization and each independently represent 1 to 20; and w11 and w14 are average values representing the numbers of CF₂ and each independently represent 1 to 2).

12. The fluorine-containing ether compound according to claim 1, wherein n in the formula (1) is 0.

13. The fluorine-containing ether compound according to claim 1, wherein n in the formula (1) is 1.

14. The fluorine-containing ether compound according to claim 1, wherein n in the formula (1) is 2.

15. The fluorine-containing ether compound according to claim 1, wherein the fluorine-containing ether compound represented by the formula (1) is any of compounds represented by following formulae (1A) to (1T) and (2A) to (2E),
Rf₁ is perfluoropolyether chain chain represented by a following formula (6-1A), and
Rf₂ is the perfluoropolyether chain represented by a following formula (6-2A): 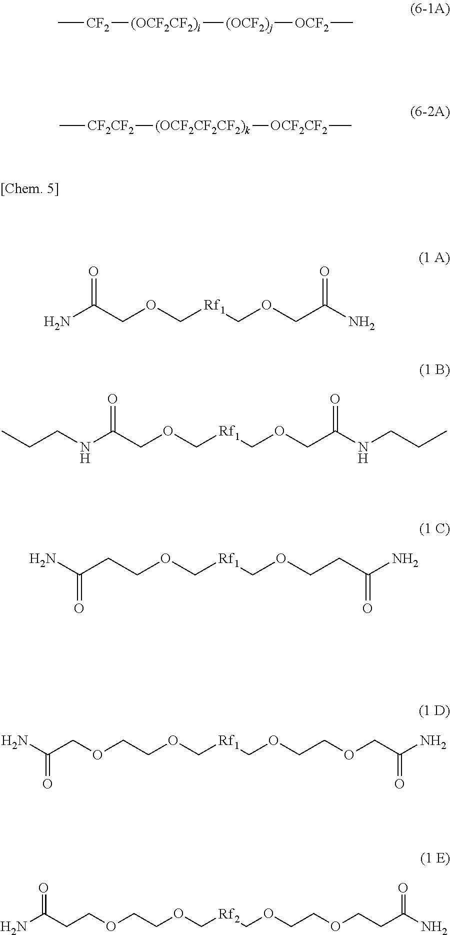
(in Rf₁ in the formula (1A), i and j indicate average degrees of polymerization; i represents 1 to 20; and j represents 0 to 20),
(in Rf₁ in the formula (1B), i and j indicate average degrees of polymerization; i represents 1 to 20; and j represents 0 to 20),
(in Rf₁ in the formula (1C), i and j indicate average degrees of polymerization; i represents 1 to 20; and j represents 0 to 20),
(in Rf₁ in the formula (1D), i and j indicate average degrees of polymerization; i represents 1 to 20; and j represents 0 to 20), and
(in Rf₂ in the formula (1E), k indicates an average degree of polymerization and represents 1 to 15) 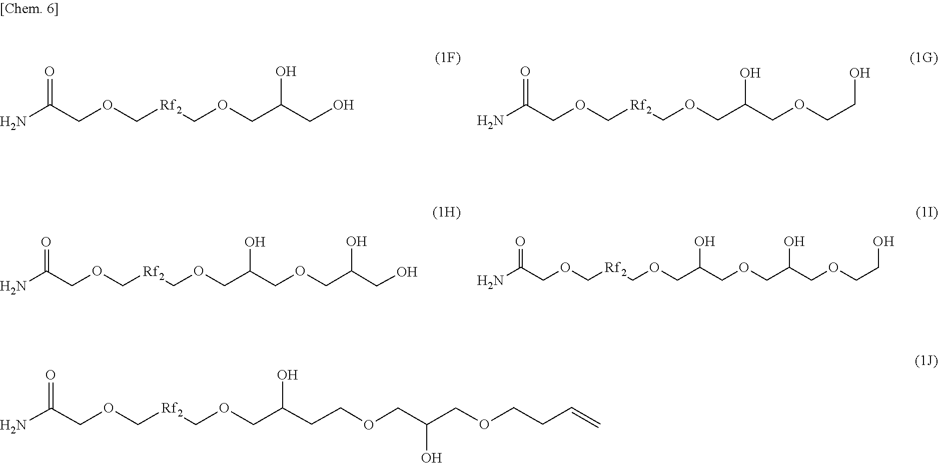
(in Rf₂ in the formula (1F), k indicates an average degree of polymerization and represents 1 to 15),
(in Rf₂ in the formula (1G), k indicates an average degree of polymerization and represents 1 to 15),
(in Rf₂ in the formula (1H), k indicates an average degree of polymerization and represents 1 to 15),
(in Rf₂ in the formula (1I), k indicates an average degree of polymerization and represents 1 to 15), and
(in Rf₂ in the formula (1J), k indicates an average degree of polymerization and represents 1 to 15) 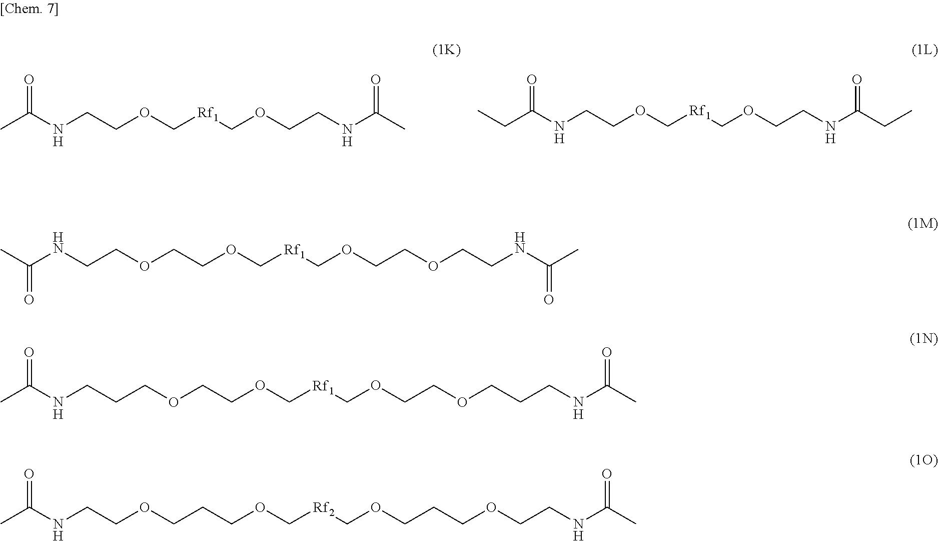
(in Rf₁ in the formula (1K), i and j indicate average degrees of polymerization; i represents 1 to 20; and j represents 0 to 20),
(in Rf₁ in the formula (1L), i and j indicate average degrees of polymerization; i represents 1 to 20; and j represents 0 to 20),
(in Rf₁ in the formula (1M), i and j indicate average degrees of polymerization; i represents 1 to 20; and j represents 0 to 20),
(in Rf₁ in the formula (1N), i and j indicate average degrees of polymerization; i represents 1 to 20; and j represents 0 to 20), and
(in Rf₂ in the formula (1O), k indicates an average degree of polymerization and represents 1 to 15) 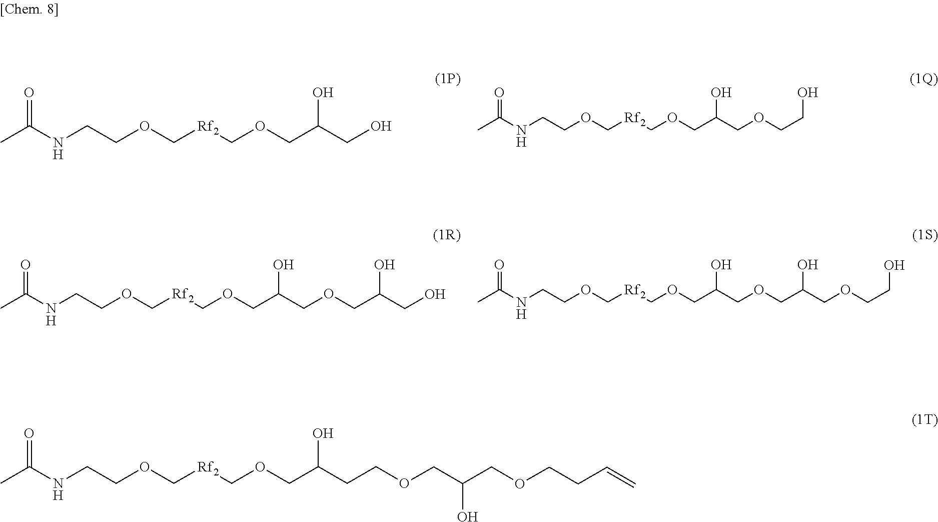
(in Rf₂ in the formula (1P), k indicates an average degree of polymerization and represents 1 to 15),
(in Rf₂ in the formula (1Q), k indicates an average degree of polymerization and represents 1 to 15),
(in Rf₂ in the formula (1R), k indicates an average degree of polymerization and represents 1 to 15),
(in Rf₂ in the formula (1S), k indicates an average degree of polymerization and represents 1 to 15), and
(in Rf₂ in the formula (1T), k indicates an average degree of polymerization and represents 1 to 15) 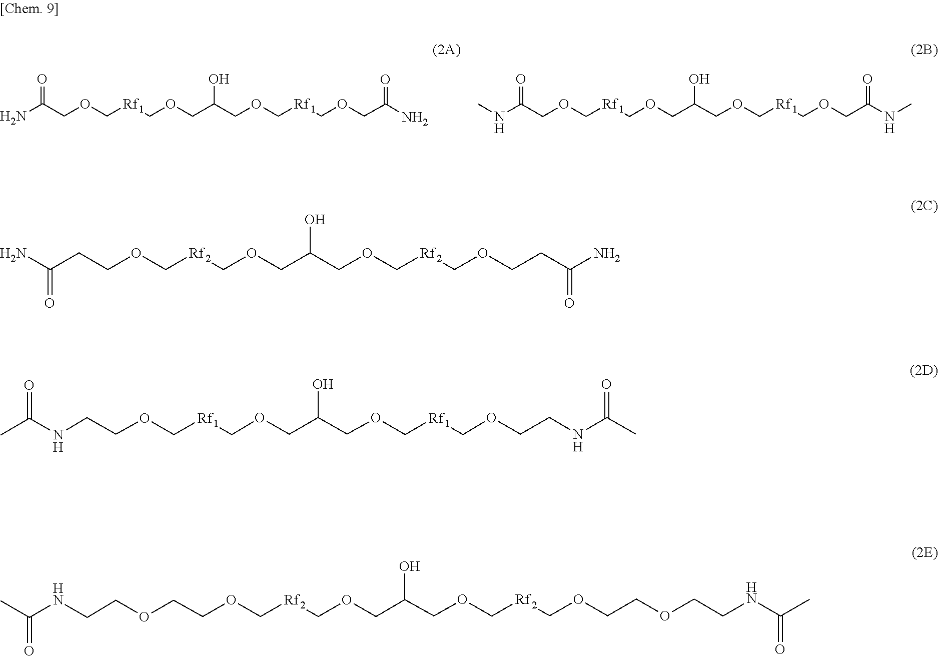
(in two Rf₁'s in the formula (2A), i and j indicate average degrees of polymerization; i represents 1 to 20; j represents 0 to 20; and in the two Rf₁'s, the average degrees of polymerization may be identical to each other or may be different from each other),
(in two Rf₁'s in the formula (2B), i and j indicate average degrees of polymerization; i represents 1 to 20; j represents 0 to 20; and in the two Rf₁'s, the average degrees of polymerization may be identical to each other or may be different from each other),
(in two Rf₂'s in the formula (2C), k indicates an average degree of polymerization and represents 1 to 15; and in the two Rf₂'s, the average degrees of polymerization may be identical to each other or may be different from each other),
(in two Rf₁'s in the formula (2D), i and j indicate average degrees of polymerization; i represents 1 to 20; j represents 0 to 20; and in the two Rf₁'s, the average degrees of polymerization may be identical to each other or may be different from each other), and
(in two Rf₂'s in the formula (2E), k indicates an average degree of polymerization and represents 1 to 15; and in the two Rf₂'s, the average degrees of polymerization may be identical to each other or may be different from each other).

16. The fluorine-containing ether compound according to claim 1, wherein a number-average molecular weight is within a range of 500 to 10000.

17. A lubricant for a magnetic recording medium comprising:
the fluorine-containing ether compound according to claim 1.

18. A magnetic recording medium comprising, at least:
a magnetic layer;
a protective layer; and
a lubricating layer provided in order on a substrate,
wherein the lubricating layer contains the fluorine-containing ether compound according to claim 1.

19. The magnetic recording medium according to claim 18, wherein the lubricating layer has an average film thickness of 0.5 nm to 2.0 nm.
